(19) United States
(12) Patent Application Publication
(10) Pub. No.: US 2022/0093251 A1
(43) Pub. Date: Mar. 24, 2022

(54) NOVEL BIOMARKERS AND DIAGNOSTIC PROFILES FOR PROSTATE CANCER

(71) Applicant: UEA ENTERPRISES LIMITED, Norwich, Norfolk (GB)

(72) Inventors: Colin Stephen Cooper, Norwich, Norfolk (GB); Jeremy Paul Clark, Norwich, Norfolk (GB); Daniel Simon Brewer, Norwich, Norfolk (GB); Shea Peter Connell, Norwich, Norfolk (GB); Helen Marie Curley, Norwich, Norfolk (GB)

(21) Appl. No.: 17/425,384

(22) PCT Filed: Jan. 28, 2020
(86) PCT No.: PCT/EP2020/052054

§ 371 Date: Jul. 23, 2021

Related U.S. Application Data
(60) Provisional application No. 62/797,437, filed on Jan. 28, 2019.

(30) Foreign Application Priority Data
Apr. 10, 2019 (GB) 1905111.9

(51) Int. Cl. G16H 50/20; G16H 50/70; G16B 25/10; C12Q 1/6886

(52) U.S. Cl. CPC G16H 50/20; G16H 50/70; C12Q 2600/158; C12Q 1/6886; G16B 25/10

(57) ABSTRACT

The present invention relates to biomarkers and diagnostic profiles based on the expression status of particular genes for use in the diagnosis of prostate cancer, in particular the early detection of prostate cancer and prediction of disease progression and Gleason ≥4 cancer. The present invention also provides methods of diagnosis and treatment of prostate cancer, and kits for the early detection of prostate cancer based on the expression status of the biomarkers in biological samples, in particular urine samples.

### FIELD OF THE INVENTION

The present invention relates to prostate cancer (PC), in particular the use of biomarkers in biological samples for the diagnosis of such conditions, such as early stage prostate cancer. The present invention also relates to the use of biomarkers in biological samples for the classification of PC, and/or as a prognostic method for predicting the disease progression of prostate cancer.

### INTRODUCTION

The progression of prostate cancer is highly heterogeneous, and risk assessment at the time of diagnosis is a critical step in the management of the disease [1]. Based on the information obtained prior to treatment, key decisions are made about the likelihood of disease progression and the best course of treatment for localised disease. D'Amico stratification [2], which classifies patients as Low- Intermediate- or High-risk of PSA-failure post-radical therapy, is based on Gleason Score (Gs) [3], PSA and clinical stage, and has been used as a framework for guidelines issued in the UK, Europe and USA [4,5,6]. Low-risk, and some favourable Intermediate-risk patients are generally offered Active Surveillance (AS) while unfavourable Intermediate-, and High-risk patients are considered for radical therapy [4,7]. Other classification systems such as CAPRA score [8] use additional clinical information, assigning simple numeric values based on age, pre-treatment PSA, Gleason Score, percentage of biopsy cores positive for cancer and clinical stage for an overall 0-10 CAPRA score. The CAPRA score has shown favourable prediction of PSA-free survival, development of metastasis and prostate cancer-specific survival [9].

The majority of prostate cancer patients are asymptomatic. Diagnosis in such cases is based on abnormalities detected by screening for serum levels of prostate-specific antigen (PSA) or findings on digital rectal examination (DRE). In addition, prostate cancer can be an incidental pathologic finding when tissue is removed during transurethral resection to manage obstructive symptoms from benign prostatic hyperplasia. Alternatively, patients may present with symptoms of primary or secondary/metastatic disease or due to the generalised effect of malignancy.

Symptoms of the primary disease are, in some cases, attributable to those originating from the prostate volume rather than cancer symptoms per se. These symptoms usually include lower urinary tract symptoms (LUTS) urine retention and or haematuria. However, patients with benign prostatic hyperplasia alone can also have similar symptoms.

Symptoms of advanced disease result from any combination of lymphatic, haematogenous, or contiguous local spread. Skeletal manifestations are especially common with more than 70% of people who die of prostate carcinoma having metastatic disease in their bones [10]. Prostate cancer has a strong capability of metastasising to bone through the haematogenous route, and symptoms will depend on the site of metastasis with manifestation as localised bone pain. The most common bones involved include those of the axial skeleton such as spine and the pelvis, although any bone may be affected. Beside bones, liver and lungs can also be affected. Lymphatic spread results in lymph node metastasis. Advanced prostate cancer can also be associated with generalised symptoms of malignancy include lethargy, weight loss and anaemia, which may be secondary to marrow infiltration or destruction by metastasis.

Diagnosis of prostate cancer is usually achieved by a combination of clinical history, examination, and investigations: clinical, histological, and radiological. Clinically a raised prostate specific antigen (PSA) and or abnormal digital rectal examination (DRE) are an indication for trans rectal biopsy of the prostate. A DRE provides a rudimentary assessment of the local extent of the tumour and clinical staging. The histological assessment provides histological grading on the disease aggressiveness. Prostatic tissue can be obtained either by the method of TRUS-guided biopsy of the prostate in patients with raised PSA or abnormal DRE that indicate the need for a biopsy or via trans-urethral resection of the prostate (TURP). According to the American Joint Committee on Cancer (AJCC) clinical staging is as follows:
T1: the tumour is present, but not detectable by DRE,
T2: the tumour can be felt (palpated) on DRE, but has not spread outside the prostate,
T3: the tumour has spread through the prostatic capsule (not detectable by DRE),
T4: the tumour has invaded other nearby structures. When a tumour has metastasised, the prostate can feel hard.

Magnetic resonance imaging (MRI), including multi-parametric magnetic resonance imaging (MP-MRI) is used in some centres in first line investigation of patients with raised PSA, followed up with a subsequent target and random biopsy in case of radiologically identifiable disease. The advantage of this is being able to identify clinically impalpable disease, anterior tumours or small foci of Gleason ≥4 and preventing biopsy-related artefacts in patients that require a post biopsy MRI for staging purposes (to assess whether the tumour is localised to within the prostate capsule, or has invaded locally, or metastasised to lymph nodes). MRI and Computer Tomography (CT) scans are typically used post-biopsy in most centres for staging. In clinically advanced disease (PSA>100 and/or locally advanced tumour on DRE) a bone nucleotide scan can be used to detect bone metastasis.

Histologically, Gleason's grading system is by far the most common prostate cancer grading method accepted and widely used. It is based on tissue architecture and the degree of tumour differentiation as identified at relatively low magnification [11]. The predominant and the second most prevalent architectural patterns are identified and assigned as grades from 1 to 5, 1 being the most differentiated, and 5 as the least differentiated. The two scores added together provide a Gleason score, which ranges from 2 to 10. Gleason grading is an independent predictor of outcome and correlates with crude survival, tumour-free survival, and cause-specific survival [12]. In addition to the Gleason grading system other microscopic features such as micro-vascular invasion and perineural infiltration can help predict the aggressiveness of the disease [13].

The prostate gland consists of three main zones, which differ histologically and biologically. The peripheral zone constitutes the bulk of the prostate, forming about 70% of the glandular part of the organ, and is the sub-capsular portion of the posterior aspect of the prostate gland that surrounds the distal urethra where its ducts open. The central zone surrounds the ejaculatory ducts and forms about 25% of the glandular prostate; its ducts open mainly into the middle prostatic urethra. The transition zone constitutes about 5% of the prostate and consists of two small lobes that surround the urethra proximal to the ejaculatory ducts. Its ducts open close to the sphincteric part of the urethra. The majority of prostate malignancies arise in the peripheral zone, which accounts for approximately 75% of all prostate cancers. The remaining 25% are found in the transition zone (20%) and central zone (5%).

Tumours in different prostatic zones have different pathological behaviours. Peripheral zone tumours are usually large in volume and are well known for their heterogeneity (Gleason scores varying from 3 to 5) and multifocality. Transition zone tumours arise in or near foci of benign prostatic hyperplasia and are smaller and better differentiated. Central zone carcinomas are the rarest, but highly aggressive with a distinct route of spread from the gland via the ejaculatory ducts and seminal vesicles routes that contrasts with spread of tumours from the other zones. Most prostate malignancies (95%) are adenocarcinoma. The remaining morphological variants are uncommon; they include ductal carcinoma variants, mucinous carcinoma, adenosquamous carcinoma and sarcomatoid carcinoma and metastases from other sites [14].

Prostate cancer is often multifocal, with disease state often underestimated by biopsy and overestimated by MP-MRI [15,16,17]. Sampling issues associated with needle biopsy of the prostate have prompted the development of non-invasive urine tests for aggressive disease which examine prostate-derived material, harvested within urine [18,19,20,21]. Certain urine biomarker tests using whole urine for predicting the presence of Gleason score (Gs) ≥7 are disclosed in references [18], [19] and [21]. The prior art tests of references [18] and [19] use PCA3 and TMPRSS2-ERG transcript expression status, whilst reference [21] uses HOXC6 and DLX1 in combination with previously identified clinical markers.

Prostate cancer has a highly unpredictable clinical behaviour which is due to its innate multifocality and heterogeneity of progression rate. Unlike most other cancers a large proportion of patients have clinically insignificant and indolent disease that will pose no real risk to their life. However due to the limitation of the available diagnostic and prognostic measures to identify aggressive prostate cancer these patients often undergo unnecessary investigation and radical treatments. This has led to the questioning of prostate cancer screening by many, as several trials have shown no significant decrease in prostate cancer-specific mortality in screened populations [22,23], while others including Schroder et al., have found a substantial reduction in PCa mortality due to PSA screening [24]. Detection of prostate cancer by PSA testing and needle biopsy alone is also unreliable as 30 to 40% of anterior tumour can be missed [25,26] as well as a significant proportion of peripheral zone tumours particularly in large prostate glands where the 10-core standard biopsy may not adequately sample the entire prostate [27].

The variation in clinical outcome for prostate cancer, and for risk stratified groups such as D'Amico, is well established. Many attempts have been made to address this problem including the subcategorisation of intermediate risk disease into favourable and unfavourable groups and the development of the CAPRA classification system. Other approaches include the development of an unsupervised classification framework and of biomarkers of aggressive disease. In each of these examples, analyses are performed on cancer biopsies, usually taken at the time of diagnosis.

A large number of prognostic biomarkers have been proposed for prostate cancer. A key question is whether these biomarkers can be applied to prostate cancer to distinguish the clinically significant cases from those with biologically irrelevant disease. Validated methods for detecting aggressive cancer early could lead to a paradigm-shift in the management of early prostate cancer.

A particular problem in the clinical management of prostate cancer is that it is highly heterogeneous. Accurate prediction of individual cancer behaviour is therefore not achievable at the time of diagnosis leading to substantial overtreatment. It remains an enigma that, in contrast to many other cancer types, stratification of prostate cancer based on unsupervised analysis of global expression patterns has not been demonstrated as effective until the recent studies defining DESNT in biopsy tissue [28].

There remains in the art a need for a more reliable diagnostic test for prostate cancer and to better assist in distinguishing between cancers of different risk levels, particularly between those with “high-risk” cancers, which may require treatment, and “low-” or “intermediate-risk” cancers, which perhaps can be kept under surveillance and left untreated to spare the patient any side effects from unnecessary interventions.

Tissue needle biopsy is an invasive technique and, in addition to the risk of infection, is associated with a degree of error in detecting clinically significant prostate cancer. Liquid biopsy is a minimally- or non-invasive technique that has gained significant traction in prospecting for novel biomarkers of urologic malignancies (PCA3, ExoDX test etc). The ductal nature of the prostate lends itself to using urine as a suitable means for sampling the prostate, both holistically and non-invasively. It has been shown that following a DRE, prostate cells, proteins and PCa specific markers such as PCA3 and the TMPRSS2:ERG gene-fusion can be detected within the urine [29,30,31,44]. Due to its minimally invasive nature, liquid biopsies have negligible morbidity when compared to TRUS biopsy [17], making urine an attractive prospect for biomarker discovery

The present invention provides an algorithm-based molecular diagnostic assay for generating one or more prostate urine risk (PUR) scores, which can be used to predict the presence or absence of cancer and/or to predict the presence of “low-” “intermediate-” or “high-” risk cancer tissue (in accordance with the criteria set out in reference 2) and/or to predict the prognosis of a prostate cancer patient. In some embodiments, the expression status of certain genes (such as those listed in Tables 1-6) may be used alone or in combination to generate a diagnostic and/or prognostic PUR score. The algorithm-based assay and associated information provided by the practice of the methods of the present invention facilitate optimal treatment decision making in prostate cancer. For example, such a clinical tool would enable physicians to identify patients who have a high risk of having aggressive disease and who therefore need radical and/or aggressive treatment.

There is an unmet need for diagnostic biomarkers that are more specific for detecting prostate cancer per se, and which can also discern indolent from clinically significant disease, particularly by relating biomarker profiles to existing risk classification scales such as D'Amico & CAPRA. Such biomarkers would retain the beneficial effect of early detection, while minimising the problems of over-diagnosis and over-treatment.

### SUMMARY OF THE INVENTION

Urine biomarkers offer the prospect of a more accurate assessment of cancer status prior to invasive tissue biopsy and may also be used to supplement standard clinical stratification using Gleason scores, Clinical Staging, PSA levels, and/or imaging techniques, such as magnetic resonance imaging (MRI). Previous urine biomarker models have been designed specifically for single purposes such as the detection of prostate cancer on re-biopsy (PCA3 test), or to detect Gs ≥7 [18,19,21].

In a first aspect of the invention, there is provided a method of providing a cancer diagnosis or prognosis based on the expression status of a plurality of genes comprising:
a) providing a plurality of patient expression profiles each comprising the expression status of the plurality of genes in at least one sample obtained from each patient, wherein each of the patient expression profiles is associated with one or more cancer risk groups, wherein each cancer risk group is associated with a different cancer prognosis or cancer diagnosis, optionally wherein each patient expression profile is normalised relative to (i) the expression status of one or more normalising genes in the same patient sample, (ii) an average expression status of one or more normalising genes in a reference population and/or (iii) the status of one or more control-probes;
b) counting the number (n) of different cancer risk groups to which the patient expression profiles belong, optionally wherein at least one cancer risk group is associated with an absence of cancer;
c) applying a cumulative link model to the patient expression profiles to select a subset of one or more genes from the plurality of genes in the patient expression profile that are significantly associated with the n cancer risk groups; and
d) inputting the expression values of the selected subset of one or more genes to a constrained continuation ratio logistic regression model comprising n modifier coefficients such that the model generates n risk scores for each patient expression profile, wherein for each patient expression profile, a risk score is provided for each of the n cancer risk groups and wherein each of the n risk scores for a given patient expression profile is associated with the likelihood of membership to the corresponding cancer risk group, optionally wherein the regression model generates regression coefficients associated with each of the selected subset of genes based on the plurality of patient expression profiles.

This method and variants thereof are hereafter referred to as Method 1.

In a second aspect of the invention, there is provided a method of classifying prostate cancer in a test subject or identifying a test subject with a poor prognosis for cancer based on the expression status of a plurality of genes comprising:
a) providing a plurality of patient expression profiles each comprising the expression status of the plurality of genes in at least one sample obtained from each patient, wherein each of the patient expression profiles is associated with one or more cancer risk groups, wherein each cancer risk group is associated with a different cancer prognosis or cancer diagnosis, optionally wherein each patient expression profile is normalised relative to (i) the expression status of one or more normalising genes in the same patient sample, (ii) an average expression status of one or more normalising genes in a reference population and/or (iii) the status of one or more control-probes;
b) counting the number (n) of different cancer risk groups to which the patient expression profiles belong, optionally wherein at least one cancer risk group is associated with an absence of cancer;
c) applying a cumulative link model to the patient expression profiles to select a subset of one or more genes from the plurality of genes in the patient expression profile that are significantly associated with the n cancer risk groups;
d) inputting the expression values of the selected subset of one or more genes to a constrained continuation ratio logistic regression model comprising n modifier coefficients such that the model generates n risk scores for each patient expression profile, wherein for each patient expression profile, a risk score is provided for each of the n cancer risk groups and wherein each of the n risk scores for a given patient expression profile is associated with the clinical outcome of the corresponding cancer risk group and wherein the regression model generates regression coefficients associated with each of the selected genes based on the plurality of patient expression profiles;
e) providing a test subject expression profile comprising the expression status of the same selected subset of one or more genes as in step (c) in at least one sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
f) inputting the test subject expression profile to the constrained continuation ratio logistic regression model comprising the n modifier coefficients and gene regression coefficients generated in step (d) to generate n risk scores for the test subject expression profile, wherein each of the n risk scores for the test subject expression profile is associated with the likelihood of membership to the corresponding cancer risk group; and
g) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

This method and variants thereof are hereafter referred to as Method 2.

In a third aspect of the invention, there is provided a method of classifying prostate cancer in a test subject or identifying a test subject with a poor prognosis for cancer comprising:
a) providing a test subject expression profile comprising the expression status of a subset of one or more genes selected by a method according to the first aspect of the invention in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
b) inputting the test subject expression profile to a constrained continuation ratio logistic regression model comprising the n modifier coefficients and gene regression coefficients generated using a method according to the first aspect of the invention, thereby generating n risk scores, wherein each of the n risk scores for a given test subject expression profile is associated with the likelihood of membership to the corresponding cancer risk group, wherein the n modifier coefficients and corresponding gene regression coefficients are generated by applying the regression model to patient expression profiles comprising the expression status of the same subset of one or more genes; and
c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

This method and variants thereof are hereafter referred to as Method 3.

In a fourth aspect of the invention, there is provided a method of classifying prostate cancer in a test subject or identifying a test subject with a poor prognosis for cancer comprising:
a) providing a test subject expression profile comprising the expression status of a plurality of the 37 genes in Table 3 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
b) inputting the test subject expression profile to a constrained continuation ratio logistic regression model comprising the 4 modifier coefficients (Cp1, Cp2, Cp3 and the intercept) and 36 gene regression coefficients in Table 8, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

This method and variants thereof are hereafter referred to as Method 4.

In a fifth aspect of the invention, there is provided a method of classifying prostate cancer in a test subject or identifying a test subject with a poor prognosis for cancer comprising:
a) providing a test subject expression profile comprising the expression status of a plurality of the 33 genes in Table 4 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
b) inputting the test subject expression profile to a constrained continuation ratio logistic regression model comprising the 4 modifier coefficients (Cp1, Cp2, Cp3 and the intercept) and 33 gene regression coefficients in Table 9, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

This method and variants thereof are hereafter referred to as Method 5.

In a sixth aspect of the invention, there is provided a method of classifying prostate cancer in a test subject or identifying a test subject with a poor prognosis for cancer comprising:
a) providing a test subject expression profile comprising the expression status of a plurality of the 29 genes in Table 5 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
b) inputting the test subject expression profile to a constrained continuation ratio logistic regression model comprising the 4 modifier coefficients (Cp1, Cp2, Cp3 and the intercept) and 29 gene regression coefficients in Table 10, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

This method and variants thereof are hereafter referred to as Method 6.

In a seventh aspect of the invention, there is provided a method of classifying prostate cancer in a test subject or identifying a test subject with a poor prognosis for cancer comprising:
a) providing a test subject expression profile comprising the expression status of a plurality of the 25 genes in Table 6 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
b) inputting the test subject expression profile to a constrained continuation ratio logistic regression model comprising the 4 modifier coefficients (Cp1, Cp2, Cp3 and the intercept) and 25 gene regression coefficients in Table 11, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

This method and variants thereof are hereafter referred to as Method 7.

In a eighth aspect of the invention, there is provided a method of classifying prostate cancer in a test subject or identifying a test subject with a poor prognosis for cancer based on the expression status of a plurality of the genes in Table 2 comprising:
a) providing a plurality of patient expression profiles each comprising the expression status of the plurality of genes in at least one sample obtained from each patient, wherein each of the patient expression profiles is associated with one of four cancer risk groups, wherein each of the four cancer risk groups is associated with (i) non-cancerous tissue, (ii) low-risk of cancer or cancer progression, (iii) intermediate-risk of cancer or cancer progression and (iv) high-risk of cancer or cancer progression; optionally wherein each patient expression profile is normalised relative to (i) the expression status of one or more normalising genes in the same patient sample, (ii) an average expression status of one or more normalising genes in a reference population and/or (iii) the status of one or more control-probes;
b) applying a cumulative link model to the patient expression profiles to select a subset of one or more genes from the plurality of genes in the patient expression profile that are significantly associated with the four cancer risk groups, optionally wherein the subset of one or more genes is the list of 37 genes in Table 3, the 29 genes in Table 5 or the 25 genes in Table 6;
c) inputting the expression values of the selected subset of one or more genes to a constrained continuation ratio logistic regression model comprising three modifier coefficients such that the model generates four risk scores for each patient expression profile, wherein for each patient expression profile, a risk score is provided for each of the four cancer risk groups and wherein each of the four risk scores for a given patient expression profile is associated with the likelihood of membership to the corresponding cancer risk group and wherein the regression model generates regression coefficients associated with each of the selected genes based on the plurality of patient expression profiles;
d) providing a test subject expression profile comprising the expression status of the same selected subset of one or more genes as in step (c) in at least one sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
e) inputting the test subject expression profile to the constrained continuation ratio logistic regression model comprising the three modifier coefficients and gene regression coefficients generated in step (d) to generate four risk scores (PUR-1, PUR-2, PUR-3 and PUR-4) for the test subject expression profile, wherein each of the four risk scores for the test subject expression profile is associated with the likelihood of membership to the corresponding cancer risk group (i) non-cancerous tissue (PUR-1), (ii) low-risk of cancer or cancer progression (PUR-2), (iii) intermediate-risk of cancer or cancer progression (PUR-3) and (iv) high-risk of cancer or cancer progression (PUR-4); and
f) determining the presence or absence of cancer in the test subject, classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

This method and variants thereof are hereafter referred to as Method 8.

In some embodiments of methods 1 and 2, the plurality of genes in step (a) comprise at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450 or 500 genes.

In some embodiments of methods 1 and 2, the plurality of genes in step (a) are selected from the genes in Table 2.

In some embodiments of methods 1, 2 and 3, the selected subset of genes comprises one or more genes (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166 or 167 genes) from the list in Table 2.

In some embodiments of methods 1, 2, 3, 4, 5, 6, 7 and 8 the at least one normalising gene is a prostate specific gene (such as those in Table 13) or a constitutively expressed housekeeping gene (such as those in Table 14).

In some embodiments of methods 1, 2, 3, 4, 5, 6, 7 and 8 the average expression status of at least one normalising gene in a reference population is the median, mean or modal expression status of the at least one normalising gene in a patient population or population of individuals without prostate cancer (for example a population of at least 50, 100, 200, 300, 400, 500, 600, 700, 800, 900, 1000, 2000, 3000, 4000, 5000, 6000, 7000, 8000, 9000 or 10000 patients or individuals).

In some embodiments of methods 1, 2, 3, 4, 5, 6, 7 and 8 the at least one normalising gene comprises 1, 2, 3, 4, 5, 6, 7, 8, 9, 10 or more normalising genes.

In a preferred embodiment of methods 1, 2, 3, 4, 5, 6, 7 and 8 the at least one normalising gene is KLK2.

In another embodiment of methods 1, 2, 3, 4, 5, 6, 7 and 8 the normalising genes are GAPDH and RPLP2.

In some embodiments of methods 1, 2, 3, 4, 5, 6, 7 and 8 the normalisation step comprises positive control normalisation.

In some embodiments of methods 1, 2, 3, 4, 5, 6, 7 and 8 the normalisation step comprises a loge transformation of expression status values.

In some embodiments of methods 1, 2, 3, 4, 5, 6, 7 and 8 the normalisation step comprises a loge transformation of positive control normalised expression status values.

In some embodiments of methods 1, 2, 3, 4, 5, 6, 7 and 8 control-probes are positive or negative control-probes, for example those supplied by NanoString® as part of the manufacturer's protocol.

In some embodiments of methods 1, 2, 3, 4, 5, 6, 7 and 8 control-probes are synthetic polynucleotides included in the determination method (e.g. microarray) to indicate that the detection of expression status of the genes of interest has either been successful (i.e. a positive control-probe).

In some embodiments of methods 1, 2, 3, 4, 5, 6, 7 and 8 the status of a control-probe within a reference population can be used to normalise an expression profile, such as a test subject expression profile.

In some embodiments of methods 1, 2 and 3, the number of cancer risk groups associated with cancer and/or absence of cancer (n) is 1, 2, 3, 4, 5, 6, 7, 8, 9 or 10.

In some embodiments of methods 1, 2, 3, 4, 5, 6, 7 and 8, the n cancer risk groups comprise a group associated with no cancer diagnosis and one or more groups (e.g. 1, 2, 3 groups) associated with increasing risk of cancer diagnosis, severity of cancer or chance of cancer progression.

In some embodiments of methods 1, 2, 3, 4, 5, 6, 7 and 8, the higher a risk score is the higher the probability a given patient or test subject exhibits or will exhibit the clinical features or outcome of the corresponding cancer risk group.

In some embodiments of methods 1, 2, 3, 4, 5, 6, 7 and 8, at least one of the cancer risk groups is associated with a poor prognosis of cancer.

In a preferred embodiment of methods 1, 2, 3, 4, 5, 6, 7 and 8, the number of cancer risk groups (n) is 4. In some embodiments of methods 1, 2, 3, 4, 5, 6, 7 and 8 the 4 cancer risk groups are the D'Amico risk groups or are equivalent to the D'Amico risk groups (i.e. no evidence of cancer, low-risk of cancer or cancer progression, intermediate-risk of cancer or cancer progression and high-risk of cancer or cancer progression).

In some embodiments of methods 1 and 2, step (c) further comprises discarding any genes that are not significantly associated with any of the n cancer risk groups.

In some embodiments of methods 1, 2, 3, 4, 5, 6, 7 and 8, the test subject expression profile is normalised against the median expression status of KLK2 in a patient population or population of individuals without prostate cancer (for example a population of at least 50, 100, 200, 300, 400, 500, 600, 700, 800, 900, 1000, 2000, 3000, 4000, 5000, 6000, 7000, 8000, 9000 or 10000 patients or individuals).

In some embodiments of method 3, the subset of one or more genes is selected from the list of genes in Table 3 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 of the genes in Table 3).

In some embodiments of method 3, the subset of one or more genes is selected from the list of genes in Table 3 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 of the genes in Table 4).

In some embodiments of method 3, the subset of one or more genes is selected from the list of genes in Table 5 (i.e. 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the genes in Table 5).

In some embodiments of method 3, the subset of one or more genes is selected from the list of genes in Table 6 (i.e. 1, 2, 3, 4, 5, 6, 7, 8, 9, 10, 11, 12, 13, 14, 15, 16, 17, 18, 19, 20, 21, 22, 23, 24 or 25 of the genes in Table 6).

In some embodiments of methods 4, 5, 6, 7 and 8, a PUR-4 score (high-risk of cancer or cancer progression) of >0.174 indicates a poor prognosis or indicates an increased likelihood of disease progression.

The invention also provides a method of diagnosing or testing for prostate cancer comprising determining the expression status of:
(i) one or more genes selected from the group consisting of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2;
(ii) one or more genes selected from the group consisting of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2;
(iii) one or more genes selected from the group consisting of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2; or
(iv) one or more genes selected from the group consisting of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2;
in a biological sample.

This method and variants thereof are hereafter referred to as Method 9.

In some embodiments of method 9 the method comprises determining the expression status of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 genes.

The terms “associated” and “correlated” are used to indicate that two or more parameters or features are related or connected in some capacity. “Associated” and “correlated” can also be used to indicate that a statistical correlation can be observed between two or more parameters. For example, the association or correlation of a particular risk score with a cancer risk group means that the level of the risk score for a given patient is directly indicative of the likelihood of that patient having a cancer diagnosis or cancer prognosis that falls into that cancer risk group.

In some embodiments of the invention the methods can be used to predict the likelihood of normal tissue, Low-risk, Intermediate risk, and/or High risk cancerous tissue being present in the prostate (e.g. based on the D'Amico scale).

In some embodiments of the invention the methods can be used to determine whether a patient should be biopsied.

In some embodiments of the invention the methods can be used to determine whether a patient should be screened using an imaging technique such as MRI (e.g. multi-parametric MRI, MP-MRI).

In some embodiments of the invention the methods are used in combination with MRI imaging data to determine whether a patient should be biopsied.

In some embodiments of the invention the MRI imaging data is generated using multiparametric MRI (MP MRI).

In some embodiments of the invention the MRI imaging data is used to generate a Prostate Imaging Reporting and Data System (PI-RADS) grade.

In some embodiments of the invention the methods can be used to predict disease progression in a patient.

In some embodiments of the invention the patient is currently undergoing or has been recommended for active surveillance.

In some embodiments of the invention the methods can be used to predict disease progression in patients with a Gleason score of ≤10, ≤9, ≤8, ≤7 or ≤6.

In some embodiments of the invention the methods can be used to predict:
(i) the volume of Gleason 4 or Gleason ≥4 prostate cancer;
(ii) significant Intermediate- or High-risk disease (based on, for example, the D'Amico grades); and/or
(iii) low risk disease that will not require treatment for at least 1, 2, 3, 4, 5 or more years.

In some embodiments of the invention the biological sample is processed prior to determining the expression status of the one or more genes in the biological sample.

In some embodiments of the invention determining the expression status of the one or more genes comprises extracting RNA from the biological sample. In some embodiments of the invention the RNA extraction step comprises chemical extraction, or solid-phase extraction, or no extraction. In some embodiments of the invention the solid-phase extraction is chromatographic extraction. In some embodiments of the invention the RNA is extracted from extracellular vesicles.

In some embodiments of the invention determining the expression status of the one or more genes comprises the step of producing one or more cDNA molecules. In some embodiments of the invention determining the expression status of the one or more genes comprises the step of quantifying the expression status of the RNA transcript or cDNA molecule. In some embodiments of the invention the expression status of the RNA or cDNA is quantified using any one or more of the following techniques: microarray analysis, real-time quantitative PCR, DNA sequencing, RNA sequencing, Northern blot analysis, in situ hybridisation, NanoString® and/or detection and quantification of a binding molecule.

In some embodiments of the invention the step of quantification of the expression status of the RNA or cDNA comprises RNA or DNA sequencing. In some embodiments of the invention the step of quantification of the expression status of the RNA or cDNA comprises using a microarray. In some embodiments of the invention the microarray analysis further comprises the step of capturing the one or more RNAs or cDNAs on a solid support and detecting hybridisation. In some embodiments of the invention the microarray analysis further comprises sequencing the one or more RNA or cDNA molecules.

In some embodiments of the invention the microarray comprises a probe having a nucleotide sequence with at least 80%, 85%, 90%, 95%, 96%, 97%, 98% or 99% identity to a nucleotide sequence selected from any one of SEQ ID NOs 1 to 76. In some embodiments of the invention the microarray comprises a probe having a nucleotide sequence selected from any one of SEQ ID NOs 1 to 76. In some embodiments of the invention the microarray comprises 74 probes, each having a unique nucleotide sequence selected from SEQ ID NOs 1 to 74.

In some embodiments of the invention the microarray comprises between 1 and 38 pairs of probes (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 pairs of probes) having a nucleotide sequence with at least 80%, 85%, 90%, 95%, 96%, 97%, 98%, 99% or 100% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74 and SEQ ID NOs 75 and 76.

In some embodiments of the invention the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, and SEQ ID NOs: 73 and 74.

In some embodiments of the invention the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, and SEQ ID NOs: 73 and 74.

In some embodiments of the invention the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74, and SEQ ID NOs: 75 and 76.

In some embodiments of the invention the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74, and SEQ ID NOs: 75 and 76.

In some embodiments of the invention the step of comparing or normalising the expression status of one or more genes with the expression status of a reference gene.

In some embodiments of the invention the expression status of a reference gene is determined in a biological sample from a healthy patient or one not known to have prostate cancer. In some embodiments of the invention the expression status of a reference gene is determined in a biological sample from a patient known to have or suspected of having prostate cancer.

In some embodiments of the invention the expression status of a reference gene is determined in a biological sample from a patient known to have Low-risk, Intermediate risk, and/or High-risk cancerous tissue (e.g. on the D'Amico scale).

In some embodiments of the invention the expression status of one or more genes of interest is compared or normalised to KLK2 as a reference gene. In some embodiments of the invention the expression status of one or more genes of interest is compared or normalised to KLK3 as a reference gene.

In some embodiments of the invention the expression status of one or more genes of interest is compared or normalised to one or more reference genes within the same test expression profile (internal normalisation). In some embodiments of the invention the expression status of one or more genes of interest is compared or normalised to the average (e.g. mean, median or modal average) of one or more reference genes within a population of expression profiles (population normalisation).

In some embodiments the step of normalisation of the expression profile to a prostate-specific gene or marker is a surrogate for normalisation to prostate volume.

In some embodiments of the invention the expression status of one or more genes of interest is compared or normalised to prostate volume, as assessed by an imaging technique such as MRI, for example MP-MRI.

In some embodiments of the invention the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample). In a preferred embodiment the biological sample is a urine sample. In some embodiments of the invention the sample is from a human. In some embodiments of the invention the biological sample is from a patient having or suspected of having prostate cancer.

In some embodiments of the invention, the sample is a urine sample collected at home. In some embodiments the urine sample is the first urine of the day or a sample taken within 1 hour of the patient waking up. In some embodiments the urine sample is taken pre-digital rectal examination (DRE). In some embodiments the urine sample is taken post-digital rectal examination (DRE). In some embodiments the urine sample is taken at multiple points throughout the day and pooled.

The invention also provides a method of treating prostate cancer, comprising diagnosing a patient as having or as being suspected of having prostate cancer using a method according to the invention, and administering to the patient a therapy for treating prostate cancer.

The invention also provides a method of treating prostate cancer in a patient, wherein the patient has been determined as having prostate cancer or as being suspected of having prostate cancer according to a method according to the invention, comprising administering to the patient a therapy for treating prostate cancer.

In some embodiments of the invention the therapy for prostate cancer comprises chemotherapy, hormone therapy, immunotherapy and/or radiotherapy. In some embodiments of the invention the chemotherapy comprises administration of one or more agents selected from the following list: abiraterone acetate, apalutamide, bicalutamide, cabazitaxel, bicalutamide, degarelix, docetaxel, leuprolide acetate, enzalutamide, apalutamide, flutamide, goserelin acetate, mitoxantrone, nilutamide, sipuleucel-T, radium 223 dichloride and docetaxel. In some embodiments of the invention the therapy for prostate cancer comprises resection of all or part of the prostate gland or resection of a prostate tumour.

The invention also provides an RNA or cDNA molecule of one or more genes selected from the group consisting of:
(i) one or more genes selected from the group consisting of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2;
(ii) one or more genes selected from the group consisting of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2;
(iii) one or more genes selected from the group consisting of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2; or
(iv) one or more genes selected from the group consisting of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2,
for use in a method of diagnosing prostate cancer comprising determining the expression status of the one or more genes.

The invention also provides a kit for testing for prostate cancer comprising a means for measuring the expression status of:
(i) one or more genes selected from the group consisting of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2;
(ii) one or more genes selected from the group consisting of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2;
(iii) one or more genes selected from the group consisting of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2; or
(iv) one or more genes selected from the group consisting of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2, in a biological sample.

In some embodiments of the invention the means for detecting is a biosensor or specific binding molecule. In some embodiments of the invention the biosensor is an electrochemical, electronic, piezoelectric, gravimetric, pyroelectric biosensor, ion channel switch, evanescent wave, surface plasmon resonance or biological biosensor

In some embodiments of the invention the means for detecting the expression status of the one or more genes is a microarray.

In some embodiments of the invention the microarray comprises specific probes that hybridise to one or more of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2.

In some embodiments of the invention the microarray comprises probes that hybridise to one or more of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2.

In some embodiments of the invention the microarray comprises probes that hybridise to one or more of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2.

In some embodiments of the invention the microarray comprises probes that hybridise to one or more of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2.

In some embodiments of the invention the kit further comprises one or more solvents for extracting RNA from the biological sample.

In embodiments of the invention, the analysis step in any of the methods can be computer implemented. The invention also provides a computer readable medium programmed to carry out any of the methods of the invention.

Constrained continuation ratio logistic regression models or general linear models can be used to produce predictors for cancer classification. The preferred approach is LASSO logistic regression analysis but alternatives such as support vector machines, neural networks, naive Bayes classifier, and random forests could be used. Such methods are well known and understood by the skilled person.

The present invention provides a method of diagnosing prostate cancer comprising generating PUR signatures that can provide a simultaneous assessment of the likelihood of non-cancerous tissue and of D'Amico Low-, Intermediate- and High-risk prostate cancer in individual prostates. The use of individual signatures for the four D'Amico risk groups is novel and can significantly aid the deconvolution of complex cancerous states into more readily identifiable forms for monitoring the development of high risk disease in, for example patients on active surveillance.

In one embodiment, the present invention provides a method of diagnosing or testing for prostate cancer.

In some embodiments, the cancer risk classifiers are the D'Amico risk classifiers [2], comprising no evidence of cancer, Low-risk, Intermediate-risk and High-risk patients, as determined by the following parameters:

No Evidence of Cancer:

No clinical signs indicating presence of prostate cancer.

Low Risk:

Clinical signs of prostate cancer and

Gleason Score <6 and

PSA <10 ng/ml and

Clinical stage T1c or T2a

Intermediate Risk:

Clinical signs of prostate cancer and

Gleason Score of 7 or

PSA of 10-20 ng/ml

Clinical stage T2b

High Risk:

Clinical signs of prostate cancer and

Gleason Score >8 or

PSA >20 ng/ml or

Clinical stage T2c or T3

The invention provides a 4-signature PUR-model capable of defining the probability of a sample containing no evidence of cancer (PUR-1), D'Amico low-risk (PUR-2), D'Amico intermediate-risk (PUR-3) and D'Amico High-risk (PUR-4) material.

For the detection of significant prostate cancer, PUR is an improvement over published biomarkers which have used simpler transcript expression systems involving low numbers of probes. The present invention demonstrates that the PUR classifier, based on the RNA expression status of 37 genes, can be used as a versatile predictor of cancer aggression. Notably PCA3, TMPRSS2-ERG and HOXC6 were all included within the original PUR gene model as defined by the LASSO criteria, while DLX1 was not. The ability of PUR-4 status to predict TRUS detected GS ≥7 is comparable (AUC, train=0.76, test=0.75) to published models using PCA3/TMPRSS2-ERG (AUC, 0.74-0.78) and HOXC6/DLX1 (AUC, 0.77).

Current clinical practice assesses patient's disease using PSA, digital rectal examination (DRE), needle biopsy of the prostate and MP-MRI. However, up to 75% of men with a raised PSA ng/ml) are negative for prostate cancer on biopsy, while 18% of tumours are found in the absence of a raised PSA, with 2% having high grade prostate cancer. This illustrates the considerable need for additional biomarkers that can make pre-biopsy assessment of prostate cancer more accurate. In this respect the present invention demonstrates that both PUR-4 and PUR-1 are each equally good at predicting the presence of intermediate or high-risk prostate cancer as defined by D'Amico criteria or by CAPRA status, while in DCA analysis the present invention demonstrates that PUR provided a net benefit in both a PSA screened and non-PSA screened populations of men.

Variation in clinical outcomes are also well recognised for patients entered onto active surveillance. We found that the PUR framework worked well when applied to men on active surveillance monitored by PSA and biopsy, and also in patients monitored by MP-MRI. Based on observations, around 13% of the Royal Marsden Hospital (RMH) active surveillance cohort could have been safely sent home and removed from AS monitoring for five years. In some patients the PUR urine signature predicted progression up to five years before it was observed with standard clinical methods. This prognostic information could potentially also aid reduction of patient-elected radical intervention in active surveillance men which in some cohorts can be as high as 75% by three years. Accordingly, in one embodiment the present invention provides a method of diagnosing prostate cancer which has a major potential clinical application.

In some embodiments the invention could be used to test which men have significant prostate cancer (Gs≥7), or whose prostate cancer has progressed to disease with a poorer prognosis, or whose disease is minimal or stable. PUR could be used as a standalone test or alongside other clinical procedures such as MRI. In some embodiments, PUR could be used to assess volume of Gleason 4 disease or Gleason ≥4. In some embodiments PUR could be used to assess how often a patient requires monitoring of their cancer status.

The present invention represents a versatile novel urine biomarker system capable of detecting significant prostate cancer (Gs≥7), and predicting disease progression in men on active surveillance. The dramatic differences in gene expression across the spectrum from high risk cancer to patients with no evidence of cancer, confirmed in a test cohort, can leave no doubt that the presence of cancer is substantially influencing the RNA transcripts found in urine EVs. The present disclosure also provides evidence that the majority of post-DRE urine EVs are derived from the prostate and that urine signatures are longitudinally stable in men whose disease has not progressed in that time frame.

### BRIEF DESCRIPTION OF THE FIGURES

FIG. 1A—PUR profiles (PUR-1, PUR-2, PUR-3, PUR-4) for the Training cohort, grouped by D'Amico risk group and ordered by ascending PUR-4 score. Horizontal lines indicate where the PUR thresholds lie for: 1° PUR-1, 2° PUR-1, 1° PUR-4, 2° PUR-4 and the crossover point between PUR-1 and PUR-4 .

FIG. 1B—PUR profiles in the Test cohort.

FIG. 1C—Examples of samples with primary PUR signatures, where circles indicate the primary PUR signal for that sample; 1° PUR-1, 1° PUR-2, 1° PUR-3, 2° PUR-4 and 1° PUR-4. The sum of all four PUR-signatures in any individual sample is 1, i.e., PUR-1+PUR-2+PUR-3+PUR-4=1.

FIG. 1D—The outline of the four PUR signatures for all samples ordered in ascending PUR-4 to illustrate where 1°, 2° and the 3° crossover point of PUR-1 and PUR-4 lie.

FIGS. 2A & B—Boxplots of PUR signatures in samples categorised as no evidence of cancer (NEC, n=62 (Training), n=30 (Test)) and D'Amico risk categories; (L—Low, n=89 (Training), n=45 (Test), I—Intermediate, n=131 (Training), n=69 (Test) and H—High risk, n=61 (Training), n=27 (Test)) in (A) the Training and (B) Test cohorts. Horizontal lines indicate where the PUR thresholds lie for: 1° PUR-1, 2° PUR-1, 1° PUR-4, 2° PUR-4,

FIGS. 2C & D—Receiver operating characteristic (ROC) curves of PUR-4 and PUR-1 predicting the presence of significant (D'Amico Intermediate or High risk) prostate cancer prior to initial biopsy in (C) Training and (D) Test cohorts. Markers indicate the specificity and sensitivity, respectively, of thresholds along the ROC curve that correspond to the indicated PUR group. For example: the PUR-4 marker and text in panel D corresponds to the PUR-4 threshold that is equivalent to a 2° PUR-1 with a specificity of 0.520 and sensitivity of 0.844 for detecting significant prostate cancer.

FIG. 3—DCA plot depicting the net benefit of adopting PUR-4 as a continuous predictor for detecting significant cancer on initial biopsy, when significant is defined as: D'Amico risk group of Intermediate or greater, GS ≥7, or Gs ≥4+3. To assess benefit in the context of cancer arising in a non-PSA screened population of men we used data from the control arm of the CAP study [64]. Bootstrap analysis with 100,000 resamples was used to adjust the distribution of Gleason grades in the Movember cohort to match that of the CAP population.

FIG. 4A—PUR profiles of patients on active surveillance that had either clinically progressed (n=23) or not (n=49) at five years post urine sample collection. Progression criteria were either: PSA velocity >1 ng/ml per year or primary Gs ≥4+3 or ≥50% cores positive for cancer on repeat biopsy. PUR signatures for progressed vs non-progressed samples were significantly different for all PUR signature (p<0.001, Wilcoxon rank sum test). Horizontal line indicates the thresholds for PUR categories described in FIG. 4B.

FIG. 4B—Kaplan-Meier plot of progression in active surveillance patients with respect to PUR categories and the number of patients within each PUR category at the given time intervals in months from urine collection.

FIG. 4C—Kaplan-Meier plot of progression with respect to the dichotomised PUR thresholds PUR-4 <0.174 and PUR-4 ≥0.174 and the number of patients within each group at the given time intervals in months from urine collection.

FIG. 5—EV-RNA yields from samples of different clinical categories collected at the NNUH. NEC—No Evidence of Cancer (n=54), L—Low risk (n=18), I—Intermediate risk (n=55), H—High risk (n=43), Post-RP—Post radical prostatectomy (n=3). Post RP and H are significantly different from all others (p<0.005 Wilcoxon-U test).

FIG. 6—Boxplots of PUR signatures relative to no evidence of cancer (NEC) and CAPRA scores 1-10 in the Training (A) and Test (B) cohorts. Numbers of samples within each group are as detailed in the table in FIG. 6B.

FIG. 7—AUC curves for each of the four PUR signatures (A) PUR-1, (B) PUR-2, (C) PUR-3, (D) PUR-4 predicting D'Amico Intermediate or High risk cancers in both training and test cohorts.

FIG. 8—AUC curves for PUR-4 predicting the presence/absence of Gs >6 in Training (A) and Test (B) cohorts and Gs >7 in Training (C) and Test (D) cohorts. Markers designate the PUR threshold at each point along the AUC curve, with number in brackets indicating the specificity and sensitivity at that threshold, respectively.

FIG. 9—DCA plot depicting the net benefit of adopting PUR-4 as a continuous predictor for detecting significant cancer on initial biopsy, when significant is defined as: D'Amico risk group of Intermediate or greater, Gs ≥7 or Gs ≥4+3. To assess benefit in the context of cancer arising with a PSA-screened population of men we used data from the intervention arm of the CAP study [64]. Bootstrap analysis was used to adjust the prevalence of Gleason grades to be representative of this population.

FIG. 10A—Kaplan-Meier plot of AS progression over time in days, including progression via MP-MRI criteria, with respect to PUR thresholds described by the corresponding colours Green—1° and 2° PUR-1, Blue—3° PUR-1, Yellow—3° PUR-4, Orange—2° PUR-4, Red—1° PUR-4. Table underneath details the number of patients still at risk of progression within each group.

FIG. 10B—Kaplan-Meier plot of progression, including progression via MP-MRI criteria, with respect to the dichotomised PUR thresholds described by the corresponding markers—PUR-4 <0.174 and—PUR-4 ≥0.174 and the number of patients within each group at the given time intervals in months from urine collection.

FIG. 11—PUR signatures in Active Surveillance longitudinal samples: PUR-1—Green, PUR-2—Blue, PUR-3—Yellow and PUR-4—Red. Samples within each numbered box are from a single patient with coloured circles underneath indicating primary PUR signature. Panel A: patients that did not reach clinical progression criteria, as described in methods. Panel B: patients that reached clinical progression criteria.

FIG. 12—A plot of PUR signatures (lower panel) and areas of Gleason 3, 4, and 5 (top panel) assessed following H&E stained slides from all blocks of radical prostatectomies in 10 patients.

FIG. 13—PUR-4 signature versus Gleason 4 tumour area for the radical prostatectomy data shown in FIG. 12. These data correspond to the numerical data in Table 12.

FIG. 14—Plots of PUR signatures versus Gleason sums for a transrectal ultrasound guided (TRUS) biopsy data set (˜650 samples). There is a trend of increasing PUR-4 with Gleason score on TRUS biopsy.

FIG. 15—Example computer apparatus.

### DETAILED DESCRIPTION OF THE INVENTION

Extracellular Vesicles

It is well documented that eukaryotic cells release extracellular vesicles including apoptotic bodies, exosomes, and other microvesicles [32,33]. Here we will use the term Extracellular Vesicle (EV) to include any membranous vesicles found in the urine such as exosomes. Extracellular vesicles differ in their cellular origins and sizes, for example, apoptotic bodies are released from the cell membrane as the final consequence of cell fragmentation during apoptosis, and they have irregular shapes with a range of 1-5 μm in size [33].

Exosomes are specialised vesicles, 30 to 100 nm in size that are actively secreted by a variety of normal and tumour cells and are present in many biological fluids, including serum and urine. They carry membrane and cytosolic components including protein and RNA into the extracellular space [34,35]. These microvesicles form as a result of inward budding of the cellular endosomal membrane resulting in the accumulation of intraluminal vesicles within large multivesicular bodies. Through this process trans-membrane proteins are incorporated into the invaginating membrane while the cytosolic components are engulfed within the intraluminal vesicles that form the exosomes, which will then be released, into the extracellular space [36,37].

So far urine exosomes have been examined in several studies for renal and prostatic pathology and have been reported to be stable in urine. RNA isolated from urine EVs had a better-preserved profile than cell-isolated RNA from the same samples [56] which makes them much better for potential biomarker use.

EV Function

EVs such as exosomes function as a means of transport for biological material between cells within an organism. As a consequence of their origin, EVs such as exosomes exhibit the mother-cell's membrane and cytoplasmic components such as proteins, lipids and genomic materials. Some of the proteins they exhibit regulate their docking and membrane fusion, for example the Rab proteins, which are the largest family of small GTPases [38]. Annexins and flotillin aid in membrane-trafficking and fusion events [39]. Exosomes also contain proteins that have been termed exosomal-marker-proteins, for example Alix, TSG101, HSP70 and the tetraspanins CD63, CD81 and CD9. Exosome protein composition is very dependent on the cell type of origin. So far a total of 13,333 exosomal proteins have been reported in the ExoCarta database, mainly from dendritic, normal and malignant cells.

Besides proteins, 2,375 mRNAs and 764 microRNAs have been reported (Exocarta.org) which can be delivered to recipient cells. Exosomes are rich in lipids such as cholesterol, sphingolipids, ceramide and glycerophospolipids which play an important role in exosome biogenesis, especially ILV formation.

EVs in Malignancy

The role of EVs such as EVs in cancer remains to be fully elucidated; they appear to function as both pro- and anti-tumour effectors. Either way cancer cell-derived EVs appear to have distinct biologic roles and molecular profiles. They can have unique gene expression signatures (RNAs, mRNAs) and proteomics profiles compared to EVs from normal cells [40,41]. Reference 40 reports large numbers of differentially expressed RNAs in EVs from melanocytes compared with melanoma-derived EVs. This indicates that exosomal RNAs may contribute to important biological functions in normal cells, as well as promoting malignancy in tumour cells. Reference 40 also suggests that cancer cell-derived EVs have a closer relationship to the originating cancer cell than normal cell derived EVs do to a normal cell, which highlights the potential of using EVs as a source of diagnostic biomarkers. RNA expression in melanoma EVs has been linked to the advancement of the disease supporting the idea that EVs such as exosomes can promote tumour growth. A similar finding was reported in glioblastoma, highlighting their potential as prognostic markers.

Experiments in mice have shown that cancer-derived EVs can induce an anti-tumour immune response. It has been demonstrated that EVs such as exosomes isolated from malignant effusions are an effective source of tumour antigens which are used by the host to present to CD8+ cytotoxic T cells, dramatically increasing the anti-tumour immune response.

EVs and Prostate Cancer

Several studies have examined the role of EVs such as exosomes in prostate cancer. Reference 42 suggests that prostate cancer derived EVs can stimulate fibroblast activation and lead to cancer development by increasing cell motility and preventing cell apoptosis. Similarly, vesicles from activated fibroblasts are, in turn, able to induce migration and invasion in the PC3 cell line. Another study reported that EVs from hormone refractory PC cells are able to induce osteoblast differentiation via the Ets1 which they contained, suggesting a role for vesicles in cell-to-cell communication during the osteoblastic metastasis process. Cell-to-cell communication was also emphasised in another study that showed that vesicles released from the human prostate carcinoma cell line DU145 are able to induce transformation in a non-malignant human prostate epithelial cell line.

Besides the in vivo evidence on the active role of EVs in cancer and cancer metastasis, Reference 43 suggests that EVs are present in high levels in the urine of cancer patients, and that unlike cells, EVs have remarkable stability in urine [44]. Other studies suggest the presence of EVs in prostatic secretions, identifying them as a potential source of prostate cancer biomarkers.

Using a nested PCR-based approach, the authors of reference 45 suggest that tumour EVs are harvestable from urine samples from PC patients and that they carry biomarkers specific to PC including KLK3, PCA3 and TMPRSS2/ERG RNAs. PCA3 transcripts were detectable in all patients including subjects with low grade disease, however TMPRSS2/ERG transcripts were only detectable in high Gleason grades. They also demonstrated in this study that i) mild prostate massage increased the extracellular vesicle secretion into the urethra and subsequently into the collected urine fraction ii) that tumour EVs are distinct from EVs shed by normal cells, and iii) they are more abundant in cancer patients.

In the present invention the RNA may be harvested from all extracellular vesicles (EV) present in urine that are below 0.8 μm. The EVs will consist of exosomes and other extracellular vesicles. In further embodiments of the invention different subtypes of EVs may be harvested and analysed.

In some embodiments of the invention RNA is extracted from urine supernatant. In some embodiments of the invention RNA is extracted from whole urine.

Apparatus and Media

The present invention also provides an apparatus configured to perform any method of the invention.

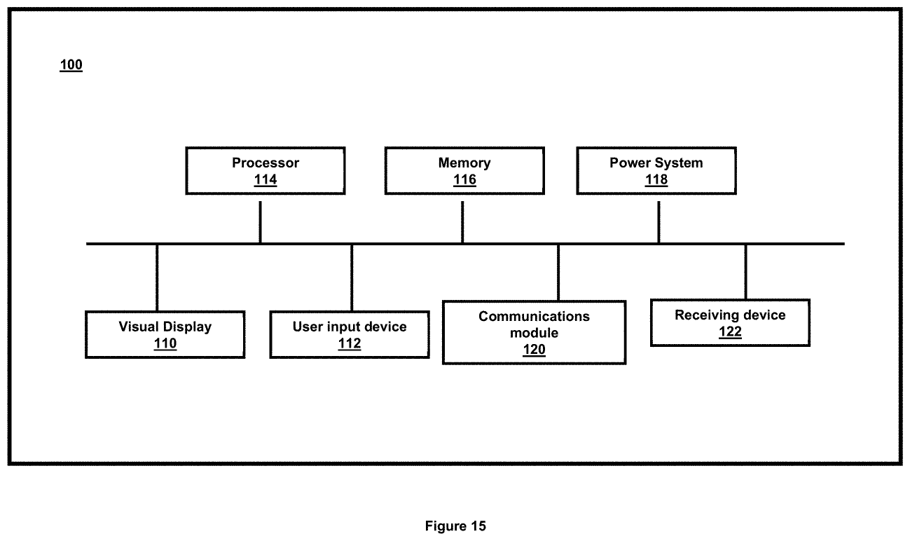

FIG. 15 shows an apparatus or computing device 100 for carrying out a method as disclosed herein. Other architectures to that shown in FIG. 15 may be used as will be appreciated by the skilled person.

Referring to the Figure, the meter 100 includes a number of user interfaces including a visual display 110 and a virtual or dedicated user input device 112. The meter 100 further includes a processor 114, a memory 116 and a power system 118. The meter 100 further comprises a communications module 120 for sending and receiving communications between processor 114 and remote systems. The meter 100 further comprises a receiving device or port 122 for receiving, for example, a memory disk or non-transitory computer readable medium carrying instructions which, when operated, will lead the processor 114 to perform a method as described herein.

The processor 114 is configured to receive data, access the memory 116, and to act upon instructions received either from said memory 116, from communications module 120 or from user input device 112. The processor controls the display 110 and may communicate date to remote parties via communications module 120.

The memory 116 may comprise computer-readable instructions which, when read by the processor, are configured to cause the processor to perform a method as described herein.

The present invention further provides a machine-readable medium (which may be transitory or non-transitory) having instructions stored thereon, the instructions being configured such that when read by a machine, the instructions cause a method as disclosed herein to be carried out.

Active Surveillance

Active surveillance (AS) is a means of disease-management for men with localised PCa with the intent to intervene if the disease progresses. AS is offered as an option to men whose prostate cancer is thought to have a low risk of causing harm in the absence of treatment. It is a chance to delay or avoid aggressive treatment such as radiotherapy or surgery, and the associated morbidities of these treatments. Entry criteria for men to go on active surveillance varies widely and can include men with Low risk and Intermediate risk prostate cancer.

Patients on AS are currently monitored by a wide range of means that include, for example, PSA monitoring, biopsy and repeat biopsy and MP-MRI. The timing of repeat biopsies, PSA testing and MP-MRI varies with the hospital, and a widely accepted method for monitoring men on AS has not yet been achieved.

In some embodiments, active surveillance comprises assessment of a patient by PSA monitoring, biopsy and repeat biopsy and/or imaging techniques such as MRI, for example MP-MRI. In some embodiments, active surveillance comprises assessment of a patient by any means appropriate for diagnosing or prognosing prostate cancer.

In some embodiments of the invention, active surveillance comprises assessment of a patient at least every 1 month, 2 months, 3 months, 4 months, 5 months, 6 months, 7 months, 8 months, 9 months, 10 months, 11 months or 12 months.

In some embodiments of the invention, active surveillance comprises assessment of a patient at least every 1 year, 2 years, 3 years, 4 years or 5 or more years.

In some embodiments of the invention the PUR signature will be used alone or in conjunction with other means of testing to improve shared decision making with the multi-disciplinary team and the patient. The PUR signature could be used to decide whether radical intervention is necessary, or to decide the optimal time between re-monitoring by, for example, biopsy, PSA testing or MP-MRI.

Biological Samples

In the present invention, the biological sample may be a urine sample, a semen sample, a prostatic exudate sample, or any sample containing macromolecules or cells originating in the prostate, a whole blood sample, a serum sample, saliva, or a biopsy (such as a prostate tissue sample or a tumour sample), although urine samples are particularly useful. The method may include a step of obtaining or providing the biological sample, or alternatively the sample may have already been obtained from a patient, for example in ex vivo methods.

Biological samples obtained from a patient can be stored until needed. Suitable storage methods include freezing immediately, within 2 hours or up to two weeks after sample collection. Maintenance at −80° C. can be used for long-term storage. Preservative may be added, or the urine collected in a tube containing preservative. Urine plus preservative such as Norgen urine preservative, can be stored between room temperature and −80° C.

Methods of the invention may comprise steps carried out on biological samples. The biological sample that is analysed may be a urine sample, a semen sample, a prostatic exudate sample, or any sample containing macromolecules or cells originating in the prostate, a whole blood sample, a serum sample, saliva, or a biopsy (such as a prostate tissue sample or a tumour sample). Most commonly for prostate cancer the biological sample is from a prostate biopsy, prostatectomy or TURP. The method may include a step of obtaining or providing the biological sample, or alternatively the sample may have already been obtained from a patient, for example in ex vivo methods. The samples are considered to be representative of the expression status of the relevant genes in the potentially cancerous prostate tissue, or other cells within the prostate, or microvesicles produced by cells within the prostate or blood or immune system. Hence the methods of the present invention may use quantitative data on RNA produced by cells within the prostate and/or the blood system and/or bone marrow in response to cancer, to determine the presence or absence of prostate cancer.

The methods of the invention may be carried out on one test sample from a patient. Alternatively, a plurality of test samples may be taken from a patient, for example at least 2, 3, 4 or 5 samples. Each sample may be subjected to a separate analysis using a method of the invention, or alternatively multiple samples from a single patient undergoing diagnosis could be included in the method.

The sample may be processed prior to determining the expression status of the biomarkers. The sample may be subject to enrichment (for example to increase the concentration of the biomarkers being quantified), centrifugation or dilution. In other embodiments, the samples do not undergo any pre-processing and are used unprocessed (such as whole urine).

In some embodiments of the invention, the biological sample may be fractionated or enriched for RNA prior to detection and quantification (i.e. measurement). The step of fractionation or enrichment can be any suitable pre-processing method step to increase the concentration of RNA in the sample or select for specific sources of RNA such as cells or extracellular vesicles. For example, the steps of fractionation and/or enrichment may comprise centrifugation and/or filtration to remove cells or unwanted analytes from the sample, or to increase the concentration of EVs in a urine fraction. Methods of the invention may include a step of amplification to increase the amount of gene transcripts that are detected and quantified. Methods of amplification include RNA amplification, amplification as cDNA, and PCR amplification. Such methods may be used to enrich the sample for any biomarkers of interest.

Generally speaking, the RNAs will need to be extracted from the biological sample. This can be achieved by a number of suitable methods. For example, extraction may involve separating the RNAs from the biological sample. Methods include chemical extraction and solid-phase extraction (for example on silica columns). Preferred methods include the use of a silica column. Methods comprise lysing cells or vesicles (if required), addition of a binding solution, centrifugation in a spin column to force the binding solution through a silica gel membrane, optional washing to remove further impurities, and elution of the nucleic acid. Commercial kits are available for such methods, for example from Qiagen or Exigon.

If RNAs are extracted from a sample, the extracted solution may require enrichment to increase the relative abundance of RNA transcripts in the sample.

The methods of the invention may be carried out on one test sample from a patient. Alternatively, a plurality of test samples may be taken from a patient, for example at least 2, at least 3, at least 4 or at least 5 samples. Each sample may be subjected to a single assay to quantify one of the biomarker panel members, or alternatively a sample may be tested for all of the biomarkers being quantified.

### Methods of the Invention

Expression Status

Determining the expression status of a gene may comprise determining the level of expression of the gene. Expression status and levels of expression as used herein can be determined by methods known to the skilled person. For example, this may refer to the up or down-regulation of a particular gene or genes, as determined by methods known to a skilled person. Epigenetic modifications may be used as an indicator of expression, for example determining DNA methylation status, or other epigenetic changes such as histone marking, RNA changes or conformation changes. Epigenetic modifications regulate expression of genes in DNA and can influence efficacy of medical treatments among patients. Aberrant epigenetic changes are associated with many diseases such as, for example, cancer. DNA methylation in animals influences dosage compensation, imprinting, and genome stability and development. Methods of determining DNA methylation are known to the skilled person (for example methylation-specific PCR, matrix-assisted laser desorption/ionization time-of-flight mass spectrometry, use of microarrays, reduced representation bisulfate sequencing (RRBS) or whole genome shotgun bisulfate sequencing (WGBS). In addition, epigenetic changes may include changes in conformation of chromatin.

Expression Analysis

NanoString® technology is based on double hybridisation of two adjacent ˜50 bp probes to their target RNA/cDNA. The first probe hybridisation is used to pull the target RNA/cDNA down on to a hard surface. The excess unbound nucleic acid is then washed away. The second probe is then hybridised to the RNA/cDNA. This probe has a multi-colour barcode attached to it. The nucleotides are then stretched out under an electrical current, and the image is recorded. The barcodes number and type are counted, and this is the data output. Up to 800 different barcodes are possible, and therefore up to 800 different target RNAs can be detected in a single assay.

Methods of real-time qPCR may involve a step of reverse transcription of RNA into complementary DNA (cDNA). PCR amplification can use sequence specific primers or combinations of other primers to amplify RNA species of interest. Microarray analysis may comprise the steps of labelling RNA or cDNA, hybridisation of the labelled RNAs to DNA (or RNA or LNA) probes on a solid-substrate array, washing the array, and scanning the array.

RNA sequencing is another method that can benefit from RNA enrichment, although this is not always necessary. RNA sequencing techniques generally use next generation sequencing methods (also known as high-throughput or massively parallel sequencing). These methods use a sequencing-by-synthesis approach and allow relative quantification and precise identification of RNA sequences. In situ hybridisation techniques can be used on tissue samples, both in vivo and ex vivo.

In some methods of the invention, detection and quantification of cDNA-binding molecule complexes may be used to determine RNA expression. For example, RNA transcripts in a sample may be converted to cDNA by reverse-transcription, after which the sample is contacted with binding molecules specific for the RNAs being quantified, detecting the presence of a of cDNA-specific binding molecule complex, and quantifying the expression of the corresponding gene. There is therefore provided the use of cDNA transcripts corresponding to one or more of the RNAs of interest, or combinations thereof, for use in methods of detecting, diagnosing or predicting prognosis of prostate. In some embodiments of the invention, the method may therefore comprise a step of conversion of the RNAs to cDNA to allow a particular analysis to be undertaken and to achieve RNA quantification.

DNA and RNA arrays (microarrays) for use in quantification of the mRNAs of interest comprise a series of microscopic spots of DNA or RNA sequences, each with a unique sequence of nucleotides that are able to bind complementary nucleic acid molecules. In this way the oligonucleotides are used as probes to which only the correct target sequence will hybridise under high-stringency condition. In the present invention, the target sequence can be the coding DNA sequence or unique section thereof, corresponding to the RNA whose expression is being detected. Most commonly the target sequence is the RNA biomarker of interest itself.

Capture molecules include antibodies, proteins, aptamers, nucleic acids, biotin, streptavidin, receptors and enzymes, which might be preferable if commercial antibodies are not available for the analyte being detected. Capture molecules for use on the arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two. The appropriate capture molecule will depend on the nature of the target (e.g. RNA, protein or cDNA).

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltammetry and amperometry methods) and radio frequency methods (for example multipolar resonance spectroscopy).

Once the expression status or concentration has been determined, the level can be compared to a threshold level or previously measured expression status or concentration (either in a sample from the same subject but obtained at a different point in time, or in a sample from a different subject, for example a healthy subject, i.e. a control or reference sample) to determine whether the expression status or concentration is higher or lower in the sample being analysed. Hence, the methods of the invention may further comprise a step of correlating said detection or quantification with a control or reference to determine if prostate cancer is present (or suspected) or not. Said correlation step may also detect the presence of a particular type, stage, grade or risk group of prostate cancer and to distinguish these patients from healthy patients, in which no prostate cancer is present or from men with indolent or low risk disease. For example, the methods may detect early stage or low risk prostate cancer. Said step of correlation may include comparing the amount (expression or concentration) of one, two, or three or more of the panel biomarkers with the amount of the corresponding biomarker(s) in a reference sample, for example in a biological sample taken from a healthy patient. The methods of the invention may include the steps of determining the amount of the corresponding biomarker in one or more reference samples which may have been previously determined. Alternatively, the method may use reference data obtained from samples from the same patient at a previous point in time. In this way, the effectiveness of any treatment can be assessed and a prognosis for the patient determined.

Internal controls can be also used, for example quantification of one or more different RNAs not part of the biomarker panel. This may provide useful information regarding the relative amounts of the biomarkers in the sample, allowing the results to be adjusted for any variances according to different populations or changes introduced according to the method of sample collection, processing or storage.

Methods of normalisation can involve correction of the counts of the measured levels of NanoString® gene-probes in order to account for, for example; differences in the input amount of RNA, variability in RNA quality and to centre data around RNA originating from prostatic material, so that all the genes being analysed are on a comparable scale.

As would be apparent to a person of skill in the art, any measurements of analyte concentration or expression may need to be normalised to take in account the type of test sample being used and/or and processing of the test sample that has occurred prior to analysis. Data normalisation also assists in identifying biologically relevant results. Invariant RNAs/mRNAs may be used to determine appropriate processing of the sample. Differential expression calculations may also be conducted between different samples to determine statistical significance. In some embodiments of the invention the expression status of KLK2 and/or KLK3 can be used for normalisation. In some embodiments of the invention the expression status of GAPDH and/or RPLP2 can be used for normalisation. In a preferred embodiment of the invention, the expression status of KLK2 is used for normalisation.

### Further Analytical Methods Used in the Invention

The expression status of a gene or protein from a biomarker panel of the invention can be determined in a number of ways. Levels of expression may be determined by, for example, quantifying the biomarkers by determining the concentration of protein in the sample, if the biomarkers are expressed as a protein in that sample. Alternatively, the amount of RNA or protein in the sample (such as a tissue sample) may be determined. Once the expression status has been determined, the level can optionally be compared to a control. This may be a previously measured expression status (either in a sample from the same subject but obtained at a different point in time, or in a sample from a different subject or subjects, for example one or more healthy subjects or one or more subjects with non-aggressive cancer, i.e. a control or reference sample) or to a different protein or peptide or other marker or means of assessment within the same sample to determine whether the expression status or protein concentration is higher or lower in the sample being analysed. Housekeeping genes can also be used as a control. Ideally, controls are one or more RNA, protein or DNA markers that generally do not vary significantly between samples or between tissue from different people or between normal tissue and tumour.

Other methods of quantifying gene expression include RNA sequencing, which in one aspect is also known as whole transcriptome shotgun sequencing (WTSS). Using RNA sequencing it is possible to determine the nature of the RNA sequences present in a sample, and furthermore to quantify gene expression by measuring the abundance of each RNA molecule (for example, RNA or microRNA transcripts). The methods use sequencing-by-synthesis approaches to enable high throughout analysis of samples.

There are several types of RNA sequencing that can be used, including RNA PolyA tail sequencing (there the polyA tail of the RNA sequences are targeting using polyT oligonucleotides), random-primed sequencing (using a random oligonucleotide primer), targeted sequence (using specific oligonucleotide primers complementary to specific gene transcripts), small RNA/non-coding RNA sequencing (which may involve isolating small non-coding RNAs, such as microRNAs, using size separation), direct RNA sequencing, and real-time PCR. In some embodiments, RNA sequence reads can be aligned to a reference genome and the number of reads for each sequence quantified to determine gene expression. In some embodiments of the invention, the methods comprise transcription assembly (de-novo or genome-guided).

RNA, DNA and protein arrays (microarrays) may be used in certain embodiments. RNA and DNA microarrays comprise a series of microscopic spots of DNA or RNA oligonucleotides, each with a unique sequence of nucleotides that are able to bind complementary nucleic acid molecules. In this way the oligonucleotides are used as probes to which the correct target sequence will hybridise under high-stringency condition. In the present invention, the target sequence can be the transcribed RNA sequence or unique section thereof, corresponding to the gene whose expression is being detected. Protein microarrays can also be used to directly detect protein expression. These are similar to DNA and RNA microarrays in that they comprise capture molecules fixed to a solid surface.

Methods for detection of RNA or cDNA can be based on hybridisation, for example, Northern blot, Microarrays, NanoString®, RNA-FISH, branched chain hybridisation assay, or amplification detection methods for quantitative reverse transcription polymerase chain reaction (qRT-PCR) such as TaqMan, or SYBR green product detection. Primer extension methods of detection such as: single nucleotide extension, Sanger sequencing. Alternatively, RNA can be sequenced by methods that include Sanger sequencing, Next Generation (high throughput) sequencing, in particular sequencing by synthesis, targeted RNAseq such as the Precise targeted RNAseq assays, or a molecular sensing device such as the Oxford Nanopore MinION device. Combinations of the above techniques may be utilised such as Transcription Mediated Amplification (TMA) as used in the Gen-Probe PCA3 assay which uses molecule capture via magnetic beads, transcription amplification, and hybridisation with a secondary probe for detection by, for example chemiluminescence.

RNA may be converted into cDNA prior to detection. RNA or cDNA may be amplified prior or as part of the detection.

The test may also constitute a functional test whereby presence of RNA or protein or other macromolecule can be detected by phenotypic change or changes within test cells. The phenotypic change or changes may include alterations in motility or invasion.

Commonly, proteins subjected to electrophoresis are also further characterised by mass spectrometry methods. Such mass spectrometry methods can include matrix-assisted laser desorption/ionisation time-of-flight (MALDI-TOF).

MALDI-TOF is an ionisation technique that allows the analysis of biomolecules (such as proteins, peptides and sugars), which tend to be fragile and fragment when ionised by more conventional ionisation methods. Ionisation is triggered by a laser beam (for example, a nitrogen laser) and a matrix is used to protect the biomolecule from being destroyed by direct laser beam exposure and to facilitate vaporisation and ionisation. The sample is mixed with the matrix molecule in solution and small amounts of the mixture are deposited on a surface and allowed to dry. The sample and matrix co-crystallise as the solvent evaporates.

Additional methods of determining protein concentration include mass spectrometry and/or liquid chromatography, such as LC-MS, UPLC, a tandem UPLC-MS/MS system, and ELISA methods. Other methods that may be used in the invention include Agilent bait capture and PCR-based methods (for example PCR amplification may be used to increase the amount of analyte).

Methods of the invention can be carried out using binding molecules or reagents specific for the analytes (RNA molecules or proteins being quantified). Binding molecules and reagents are those molecules that have an affinity for the RNA molecules or proteins being detected such that they can form binding molecule/reagent-analyte complexes that can be detected using any method known in the art. The binding molecule of the invention can be an oligonucleotide, or oligoribonucleotide or locked nucleic acid or other similar molecule, an antibody, an antibody fragment, a protein, an aptamer or molecularly imprinted polymeric structure, or other molecule that can bind to DNA or RNA. Methods of the invention may comprise contacting the biological sample with an appropriate binding molecule or molecules. Said binding molecules may form part of a kit of the invention, in particular they may form part of the biosensors of in the present invention.

Aptamers are oligonucleotides or peptide molecules that bind a specific target molecule. Oligonucleotide aptamers include DNA aptamer and RNA aptamers. Aptamers can be created by an in vitro selection process from pools of random sequence oligonucleotides or peptides. Aptamers can be optionally combined with ribozymes to self-cleave in the presence of their target molecule. Other oligonucleotides may include RNA molecules that are complimentary to the RNA molecules being quantified. For example, polyT oligos can be used to target the polyA tail of RNA molecules.

Aptamers can be made by any process known in the art. For example, a process through which aptamers may be identified is systematic evolution of ligands by exponential enrichment (SELEX). This involves repetitively reducing the complexity of a library of molecules by partitioning on the basis of selective binding to the target molecule, followed by re-amplification. A library of potential aptamers is incubated with the target protein before the unbound members are partitioned from the bound members. The bound members are recovered and amplified (for example, by polymerase chain reaction) in order to produce a library of reduced complexity (an enriched pool). The enriched pool is used to initiate a second cycle of SELEX. The binding of subsequent enriched pools to the target protein is monitored cycle by cycle. An enriched pool is cloned once it is judged that the proportion of binding molecules has risen to an adequate level. The binding molecules are then analysed individually. SELEX is reviewed in [46].

Statistical Analysis

Cumulative Link Model

Cumulative link models (CLMs) are used exclusively for ordinal data, where there is a specified direction or order to the possible response values [47,48]. They are also widely known as ordinal regression models, ordered probit models and ordered logit models. The most common name for a CLM with a logit link is a proportional odds model. CLMs arise from focusing on the cumulative distribution of the response variable, associating a samples probability that it is a certain category or lower.

Coefficient Modifiers

Constrained continuation ratio models incorporates coefficient modifiers to generate the corresponding number of risk scores to the number of ordinal classes into which the data is classified (e.g. cancer risk groups). Accordingly for n classes, there will be n−1 intercepts representing the value to be added for each class to the sum of all variable coefficient products before transformation via an appropriate link function. The nomenclature for these cutpoints can be “cpx” wherein x=1, x=2, x=3 . . . x=n−1. In some embodiments n=4 so the intercepts are cp1, cp2 and cp3.

PUR Signature Construction

Statistical analyses and model construction were undertaken in R version 3.4.1 [59] and unless otherwise stated, utilised base R and default parameters. The Prostate Urine Risk (PUR) signatures were constructed from the training set as follows: for each probe, a univariate cumulative link model was fitted using the R package clm with risk group as the outcome and NanoString® expression as inputs. Each probe that had a significant association with risk group (p<0.05) was used as input to the final multivariate model. A constrained continuation ratio model with an L1 penalisation was fitted to the training dataset using the glmnetcr library, an adaption of the LASSO method. Default parameters were applied using the LASSO penalty and values from all probes selected by the univariate analysis used as input. The model with the minimum Akaike information criterion was selected. Where multiple samples were analysed from the same patient, the sample with the highest PUR-4 signature was used in survival analyses and Kaplan-Meier (KM) plots.

Decision Curve Analysis (DCA)

Decision curve analysis is a method of evaluating predictive models. It assumes that the threshold probability of a disease or event at which a patient would opt for treatment is informative of how the patient weighs the relative harms of a false-positive and a false-negative prediction. This theoretical relationship is then used to derive the net benefit of the model across different threshold probabilities. Plotting net benefit against threshold probability yields the “decision curve.” Decision curve analysis can be used to identify the range of threshold probabilities in which a model is of value, the magnitude of benefit, and which of several models is optimal [66].

Kaplan Meier (KM)

Is the most common method used for estimating survival functions. Designed to deal with data that has incomplete observations using censoring. It works by using a start point and an end point for each subject. In one case, the KM analysis can be used to study survival of patients on active surveillance and the start point is when the person joins the study or the active surveillance monitoring, or a sample is collected for PUR analysis, and the end point is when subsequent progression was found for each patient or the patient has radical intervention treatment. Data is often incomplete due to patients dropping out of the study or insufficient follow up of patients, here censoring is used to ensure there is no bias. Where multiple samples were analysed from the same patient, the sample with the highest PUR-4 signature was used in survival analyses and Kaplan-Meier (KM) plots.

Gene Transcript Detection

The present invention provides probes suitable for use in cDNA or RNA sequence detection such as NanoString® or microarray techniques which can be used to determine the expression status of genes of interest. Methods of the invention can be operated using any suitable probe sequence to detect a gene transcript and methods of generating probe sequences are known to those skilled in the art.

In another embodiment the gene transcripts may be detected by sequencing, or qRT-PCR.

In some embodiments, the methods of the invention comprise a step of determining the expression status of a gene by using a probe having a nucleotide sequence selected from any one of the following sequences (Table 1):

TABLE 1
Genes of interest and associated capture probes

| | Gene | | | |
|---|---|---|---|---|
| Official | name | Accession | Capture probe | Reporter probe |
| symbol | Long | number | sequence | sequence |
| AMACR | alpha- | NM_014324.4 | TGGAATCTACCCCTTCCTCA | CAACATCCATTCTCTACTCC |
| | methylacyl- | (Accessed 5^{th} | CATGCCTTTAGGAAGTTGAG | CTCTACTCTGATGGCACCCG |
| | CoA | November 2018) | TCCAGGGAAG | GATTAGATTG |
| | racemase | | (SEQ ID NO: 1) | (SEQ ID NO: 2) |
| AMH | anti- | NM_000479.3 | TTGGCCTGGTAGGTCTCGGG | CGGACTGAGGCCAGCCGCAC |
| | Mullerian | (Accessed 5^{th} | GATGAGTACGGAGCG | ACGCCCTGGCAATTG |
| | hormone | November 2018) | (SEQ ID NO: 3) | (SEQ ID NO: 4) |
| ANKRD34B | ankyrin | NM_001004441.2 | TTTATAGGATAGTTCTTCCT | ATGCTTTGGTGCCTAGTGAT |
| | repeat | (Accessed 5^{th} | CTGGTGTAATATCCTGGAGC | GAACCGCTTGGAAAGTGCCA |
| | domain 34B | November 2018) | TCCTCTTGCA | GCCCATTGGT |
| | | | (SEQ ID NO: 5) | (SEQ ID NO: 6) |
| APOC1 | apolipoprote | NM_001645.3 | CGGAGGGGCACTCTGAATCC | CAGAACCACCACCAGGACCG |
| | in C1 | (Accessed 5^{th} | TTGCTGGAGGGCTTGGTTGG | GGAGCGACAGGAAGAGCCTC |
| | | November 2018) | GAGGTC | ATGGCGAGGC |
| | | | (SEQ ID NO: 7) | (SEQ ID NO: 8) |
| ARexons4-8 | Androgen | NM_000044.2 | GACTTGTGCATGCGGTACTC | CAAACTCTTGAGAGAGGTGC |
| | Receptor | (Accessed 5^{th} | ATTGAAAACCAGATCAGGGG | CTCATTCGGACACACTGGCT |
| | | November 2018) | CGAAGTAGAG | GTACATCCGG |
| | | | (SEQ ID NO: 9) | (SEQ ID NO: 10) |
| DPP4 | dipeptidyl | NM_001935.3 | AAATCCACTCCAACATCGAC | CTGCTAGCTATTCCATGGTC |
| | peptidase 4 | (Accessed 5^{th} | CAGGGCTTTGGAGATCTGAG | TTCATCAGTATACCACATTG |
| | | November 2018) | CTGACTGCTG | CCTGG |
| | | | (SEQ ID NO: 11) | (SEQ ID NO: 12) |
| ERG (3′ to | ERG, ETS | NM_004449.4 | TGAGCCATTCACCTGGCTAG | CCACCATCTTCCCGCCTTTG |
| usual | transcription | (Accessed 5^{th} | GGTTACATTCCATTTTGATG | GCCACACTGCATTCATCAGG |
| translocation | factor | November 2018) | GTGACCCTGG | AGAGTTCCT |
| breakpoint, | | | (SEQ ID NO: 13) | (SEQ ID NO: 14) |
| exons 4-5) | | | | |
| GABARAPL2 | GABA type A | NM_007285.6 | GGGACTGTCTTATCCACAAA | CTTCATCTTTTTCCTTCTCG |
| | receptor | (Accessed 5^{th} | CAGGAAGATCGCCTTTTCAG | TAAAGCTGTCCCATAGTTAG |
| | associated | November 2018) | AAGGAAGCTG | GCTGGACTGT |
| | protein like | | (SEQ ID NO: 15) | (SEQ ID NO: 16) |
| | 2 | | | |
| GAPDH | glyceraldehyde- | NM_002046.3 | AAGTGGTCGTTGAGGGCAAT | CCCTGTTGCTGTAGCCAAAT |
| | 3- | (Accessed 5^{th} | GCCAGCCCCAGCGTCAAAG | TCGTTGTCATACCAGGAAAT |
| | phosphate | November 2018) | (SEQ ID NO: 17) | GAGCTTGACA |
| | dehydrogenase | | | (SEQ ID NO: 18) |
| GDF15/MIC1 | growth | NM_004864.2 | CCTGGTTAGCAGGTCCTCGT | GTGTTCGAATCTTCCCAGCT |
| | differentiation | (Accessed 5^{th} | AGCGTTTCCGCAACTC | CTGGTTGGCCCGCAG |
| | factor 15 | November 2018) | (SEQ ID NO: 19) | (SEQ ID NO: 20) |
| HOXC6 | homeobox | NM_153693.3 | GGTCGAGAAATGCCTCACTG | GAATAAAAGGGAGTCGAGTA |
| | C6 | (Accessed 5^{th} | GATCATAGGCGGTGGAATTG | GATCCGGTTCTGGGCAACGG |
| | | November 2018) | AGGGCGACGT | CCGCTCCATA |
| | | | (SEQ ID NO: 21) | (SEQ ID NO: 22) |
| HPN | hepsin | NM_182983.1 | CCGAGAGATGCTGTCCTCAC | CCAACTCACAATGCCACACA |
| | | (Accessed 5^{th} | ACACAAAGGGACCACCGCTG | GCCGCCAACGTGGCGT |
| | | November 2018) | (SEQ ID NO: 23) | (SEQ ID NO: 24) |
| IGFBP3 | insulin like | NM_000598.4 | CGGGCGCATGAAGTCTGGGT | TGGTCGGCCGCTTCGACCAA |
| | growth | (Accessed 5^{th} | GCTGTGCTCGAGTCTCTGAA | CATGTGGTGAGCATTCCA |
| | factor | November 2018) | TATTTTGATA | (SEQ ID NO: 26) |
| | binding | | (SEQ ID NO: 25) | |
| | protein 3 | | | |
| IMPDH2 | inosine | NM_000884.2 | TCTTTGAGAAAATCAATGTC | TCCCTCTTTGTCATTATCTC |
| | monophosphate | (Accessed 5^{th} | CCTGGAGGAGATGATGCCCA | TTCCAAGAAACAGTCATGTT |
| | dehydrogenase 2 | November 2018) | CCAAGCGGCT | CCTCC |
| | | | (SEQ ID NO: 27) | (SEQ ID NO: 28) |
| ITGBL1 | integrin | NM_004791.2 | AGACCACACCATCGAGGTCT | TCCTCTCTCACAAACACAGC |
| | subunit beta | (Accessed 5^{th} | TCACAGCGGCGATCATCACA | GACCACAGGAACATGTGCCG |
| | like 1 | November 2018) | CTCACAAGTC | TGGCCTCCAC |
| | | | (SEQ ID NO: 29) | (SEQ ID NO: 30) |
| KLK2 | kallikrein | NM_005551.3 | CTTGGACACTAAGGATCAGG | GTCAATTATTCAAGTACTCC |
| | related | (Accessed 5^{th} | TGAGCTTCCTCAGTTGGAAT | ATACTCGTCCTACAGACCCC |
| | peptidase 2 | November 2018) | TACTTTGTAC | CAGTAAAAAC |
| | | | (SEQ ID NO: 31) | (SEQ ID NO: 32) |
| KLK4 | kallikrein | NM_004917.3 | CCCAGCCAGAAACGAGGCAA | CAGCACGGTAGGCATTCTGC |
| | related | (Accessed 5^{th} | GAGTTCCCCGCGGTAG | CGTTCGCCAGCAGAC |
| | peptidase 4 | November 2018) | (SEQ ID NO: 33) | (SEQ ID NO: 34) |
| MARCH5 | membrane | NM_017824.4 | TGTGCTGAAACTAGACTGTC | AAACAAAGAGCTCAAGGCCT |
| | associated | (Accessed 5^{th} | AACTCTGTAAGAGCTTGGAC | CACCTTGGTTTATTCACTGC |
| | ring-CH-type | November 2018) | CAAGTCTGTC | TGGTTTTCTA |
| | finger 5 | | (SEQ ID NO: 35) | (SEQ ID NO: 36) |
| MED4 | mediator | NM_001270629.1 | TCTTGCTTTTTCTATTGACT | CTGATCCTATGTGCATACTT |
| | complex | (Accessed 5^{th} | TGAGTTTCTCCTTCGCTTGG | AATTATTTCTTCAGAGGAGA |
| | subunit 4 | November 2018) | TAAACAGCTG | TAGCACCTTT |
| | | | (SEQ ID NO: 37) | (SEQ ID NO: 38) |
| MEMO1 | mediator of | NM_001137602.1 | GAATGTGCAGGTGGCATCCC | TATCGTGGTAAAGGCTAGGC |
| | cell motility | (Accessed 5^{th} | TGAGGATTCAGAGCT | TGGGACCCCGGACAGAGTAT |
| | 1 | November 2018) | (SEQ ID NO: 39) | GA (SEQ ID NO: 40) |
| MEX3A | mex-3 RNA | NM_001093725.1 | GATCTATGCAACTTCTGATA | CCTTTCAGCCACAGAAACGA |
| | binding | (Accessed 5^{th} | GGACTCCAACTCCCTTACAC | TTGACATGCTTCTCTCCCCA |
| | family | November 2018) | TGCTGGAAAC | ACCCCTAGAA |
| | member A | | (SEQ ID NO: 41) | (SEQ ID NO: 42) |
| MME/CD10 | membrane | NM_000902.2 | TAGGGCTGGAACAAGGACTC | CCAAAGGAATATTGCAAATA |
| | metalloendo | (Accessed 5^{th} | TTTTCTCTGGACAGCTTGCA | CCCAAGGTCACCCTGTCAGG |
| | peptidase | November 2018) | CCTACAATCC | AGTGGCAGAA |
| | | | (SEQ ID NO: 43) | (SEQ ID NO: 44) |
| MMP11 | matrix | NM_005940.3 | TCAGTGGGTAGCGAAAGGTG | ATATAGGTGTTGAACGCCCC |
| | metallopeptidase | (Accessed 5^{th} | TAGAAGGCGGACATCAGGGC | TGCAGTCATCTGGGCTGAGA |
| | 11 | November 2018) | CTTGG (SEQ ID NO: 45) | C (SEQ ID NO: 46) |
| MMP26 | matrix | NM_021801.3 | CAGGATTTCCAGAATTTGGT | TCCAGTGTCTGAAGCTGACC |
| | metallopeptidase | (Accessed 5^{th} | AAAAAGGCATGGCCTAAGAT | AGTGTTCATTCTTGTCAAAA |
| | 26 | November 2018) | ACCACCTGGC | TGGACAACTC |
| | | | (SEQ ID NO: 47) | (SEQ ID NO: 48) |
| NKAIN1 | Na+/K+ | NM_024522.2 | CACTGTGTTCAAGGCCCACT | GAACTCAGAGAGCAGACACT |
| | transporting | (Accessed 5^{th} | TCCACCAAAAATCTAGCTGT | GGGTTTTACAGTCAGAAACT |
| | ATPase | November 2018) | GTGGCCTCAA | GCAGAAAGTA |
| | interacting 1 | | (SEQ ID NO: 49) | (SEQ ID NO: 50) |
| PALM3 | paralemmin | NM_001145028.1 | AGCTGGGACTGGAGTGTGAA | GCTGGGCACCTGTGGAAGCA |
| | 3 | (Accessed 5^{th} | CAAACTGTCTTCCAGGTTCC | CTTTGCAACAGTTGC |
| | | November 2018) | G (SEQ ID NO: 51) | (SEQ ID NO: 52) |
| PCA3 | prostate | NR_015342.1 | TAAGGAACACATCAATTCAT | TCCCGTTCAAATAAATATCC |
| | cancer | (Accessed 5^{th} | TTTCTAATGTCCTTCCCTCA | ACAACAGGATCTGTTTTCCT |
| | associated 3 | November 2018) | CAAGCGGGAC | GCCCATCCTT |
| | (non-protein | | (SEQ ID NO: 53) | (SEQ ID NO: 54) |
| | coding) | | | |
| PPFIA2 | PTPRF | NM_003625.2 | CACTTTCATCCAGTCGCCTT | AGGAGGAAACTGCCTTCTCC |
| | interacting | (Accessed 5^{th} | TCAGTTCCCAGGGCCAAGAG | AGGTTGATCCACGTCTGAAG |
| | protein | November 2018) | GTTATTGTAT | TTCTTGTCAT |
| | alpha 2 | | (SEQ ID NO: 55) | (SEQ ID NO: 56) |
| SIM2.short | single- | NM_005069.3 | TTAATGTAGGTCGTGCGCAT | ATCCGCAAGTCGGCGGCGGG |
| | minded | (Accessed 5^{th} | TTGCCGGGCTCGGTGGCGCC | GTCCAATTCAAACAGCTGTC |
| | family bHLH | November 2018) | GCAGCC | TCTGCATAAA |
| | transcription | | (SEQ ID NO: 57) | (SEQ ID NO: 58) |
| | factor 2 | | | |
| SMIM1 | small | ENS1000004448 | TTCATGGCGATGCCCAGCTT | GGTAGCCCAGGATGAAGATG |
| | integral | 70.1 | GCCCGTGCACAGCCTCTGGG | ATCCAGAAGAGGGCCACGCC |
| | membrane | (Accessed 5^{th} | AGAT (SEQ ID NO: 59) | GCCCAGCACC |
| | protein 1 | November 2018) | | (SEQ ID NO: 60) |
| | (Vel blood | | | |
| | group) | | | |
| SSPO | SCO-spondin | NM_198455.2 | CCACAAGGCAGGGAGAGAAG | ATGGTAGGCATCATGAAGGG |
| | | (Accessed 5^{th} | GGAGCCACATAAGTAGATTC | CACAGTGCTCGCTGC |
| | | November 2018) | CTGGCG (SEQ ID NO: 61) | (SEQ ID NO: 62) |
| SULT1A1 | sulfotransferase | NM_177534.2 | CCCTCAATTCATATTTTATT | TCAGCCTCCAAATTGCTGGG |
| | family | (Accessed 5^{th} | CTTGAGCCGCTTGGTCAGGT | ATTACAGACATGACCTACCG |
| | 1A member | November 2018) | TTGATTCGCA | TCCCGGG |
| | 1 | | (SEQ ID NO: 63) | (SEQ ID NO: 64) |
| TDRD | Tudor | NM_198795.1 | TGTTTCTAGACTGTATATCT | CCCAGCAACACACATCTGGA |
| | domain | (Accessed 5^{th} | GCTAACTGGCACCGTATTCC | ATCTTGTTATGGCTTCTTCA |
| | containing 1 | November 2018) | CTGAAAGGGA | GACCAATGTT |
| | | | (SEQ ID NO: 65) | (SEQ ID NO: 66) |
| IMPRSS2/ERG | transmembrane | Fusion_0120.1 | CTGCCGCGCTCCAGGCGGCG | TAGGCACACTCAAACAACGA |
| fusion | protease, | EU432099.1 | CTCCCCGCCCCTCGC | CTGGTCCTCACTCACAACTG |
| | serine 2/ERG | (Accessed 5^{th} | (SEQ ID NO: 67) | ATAAGGCTTC |
| | fusion | November 2018) | | (SEQ ID NO: 68) |
| TRPM4 | transient | NM_001195227.1 | CTTCCAGTAGAGATCGCTGT | GCCAGCGCGGGCCGAGAGTG |
| | receptor | (Accessed 5^{th} | TGCCCTGTACTTTGCCGAAT | GAATTCCCGGATGAGGCGGT |
| | potential | November 2018) | GTGTAACTGA | AACGCTGCGC |
| | cation | | (SEQ ID NO: 69) | (SEQ ID NO: 70) |
| | channel | | | |
| | subfamily M | | | |
| | member 4 | | | |
| TWIST1 | twist family | NM_000474.3 | CTCGGCGGCTGCTGCCGGTC | TGCTGCTGCGCCGCTTGCGT |
| | bHLH | (Accessed 5^{th} | TGGCTCTTCCTCGCTG | CCCCCGCGCTTGCCG |
| | transcription | November 2018) | (SEQ ID NO: 71) | (SEQ ID NO: 72) |
| | factor 1 | | | |
| UPK2 | uroplakin 2 | NM_006760.3 | ACGAGGTTTGTCACCTGGTA | TCCCCTTCTTCACTAGGTAG |
| | | (Accessed 5^{th} | TGCACTGAGCCGAGTGACTG | GAAATGTAGAATTTGGTTCC |
| | | November 2018) | (SEQ ID NO: 73) | TGGC |
| | | | | (SEQ ID NO: 74) |
| SLC12A1 | solute | NM_000338.2 | CCATATACAACAAATCCGAT | TCTAACTAGTAAGACAGGTG |
| | carrier | (Accessed 5^{th} | ATGGATCCCTTTCTTGCCAC | GGAGGTTCTTTGTGAGGATT |
| | family 12 | November 2018) | GGGAAGGCTC | TCCAACCAAG |
| | member 1 | | (SEQ ID NO: 75) | (SEQ ID NO: 76) |

Kits and Biosensors

In a still further embodiment of the invention there is provided a kit of parts for testing for prostate cancer comprising a means for quantifying the expression or concentration of (i.e. measuring), one or more gene transcripts selected from the group consisting of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SLC12A1, SMIM1, SSPO, SULT1A1, TDRD1, TMPRSS2:ERG, TRPM4, TWIST1 and UPK2 in a biological sample. The means may be any suitable detection means that can measure the quantity of biomarkers in the sample.

In one embodiment, the means may be a biosensor. The kit may also comprise a container for the sample or samples and/or a solvent for extracting the biomarkers from the biological sample. The kits of the present invention may also comprise instructions for use.

The kit of parts of the invention may comprise a biosensor. A biosensor incorporates a biological sensing element and provides information on a biological sample, for example the presence (or absence) or concentration of an analyte. Specifically, they combine a biorecognition component (a bioreceptor) with a physiochemical detector for detection and/or quantification of an analyte (such as an RNA, a cDNA or a protein).

The bioreceptor specifically interacts with or binds to the analyte of interest and may be, for example, an antibody or antibody fragment, an enzyme, a nucleic acid, an organelle, a cell, a biological tissue, imprinted molecule or a small molecule. The bioreceptor may be immobilised on a support, for example a metal, glass or polymer support, or a 3-dimensional lattice support, such as a hydrogel support.

Biosensors are often classified according to the type of biotransducer present. For example, the biosensor may be an electrochemical (such as a potentiometric), electronic, piezoelectric, gravimetric, pyroelectric biosensor or ion channel switch biosensor. The transducer translates the interaction between the analyte of interest and the bioreceptor into a quantifiable signal such that the amount of analyte present can be determined accurately. Optical biosensors may rely on the surface plasmon resonance resulting from the interaction between the bioreceptor and the analyte of interest. The SPR can hence be used to quantify the amount of analyte in a test sample. Other types of biosensor include evanescent wave biosensors, nanobiosensors and biological biosensors (for example enzymatic, nucleic acid (such as DNA), antibody, epigenetic, organelle, cell, tissue or microbial biosensors).

The invention also provides microarrays (RNA, DNA or protein) comprising capture molecules (such as RNA or DNA oligonucleotides) specific for each of the biomarkers or biomarker panels being quantified, wherein the capture molecules are immobilised on a solid support. The microarrays are useful in the methods of the invention.

The binding molecules may be present on a solid substrate, such an array (for example an RNA microarray, in which case the binding molecules are DNA or RNA molecules that hybridise to the target RNA or cDNA). The binding molecules may all be present on the same solid substrate. Alternatively, the binding molecules may be present on different substrates. In some embodiments of the invention, the binding molecules are present in solution.

These kits may further comprise additional components, such as a buffer solution. Other components may include a labelling molecule for the detection of the bound RNA and so the necessary reagents (i.e. enzyme, buffer, etc) to perform the labelling; binding buffer; washing solution to remove all the unbound or non-specifically bound RNAs. Hybridisation will be dependent on the size of the putative binder, and the method used may be determined experimentally, as is standard in the art. As an example, hybridisation can be performed at ˜20° C. below the melting temperature (Tm), over-night. (Hybridisation buffer: 50% deionised formamide, 0.3 M NaCl, 20 mM Tris-HCI, pH 8.0, 5 mM EDTA, 10 mM phosphate buffer, pH 8.0, 10% dextran sulfate, 1× Denhardt's solution, and 0.5 mg/mL yeast tRNA). Washes can be performed at 4-6° C. higher than hybridisation temperature with 50% Formamide/2×SSC (20×Standard Saline Citrate (SSC), pH 7.5: 3 M NaCl, 0.3 M sodium citrate, the pH is adjusted to 7.5 with 1 M HCI). A second wash can be performed with 1× PBS/0.1% Tween 20.

Binding or hybridisation of the binding molecules to the target analyte may occur under standard or experimentally determined conditions. The skilled person would appreciate what stringent conditions are required, depending on the biomarkers being measured. The stringent conditions may include a hybridisation buffer that is high in salt concentration, and a temperature of hybridisation high enough to reduce non-specific binding.

Biopsies

A prostate biopsy involves taking a sample of the prostate tissue, for example by using thin needles to take small samples of tissue from the prostate. The tissue is then examined under a microscope to check for cancer.

There are two main types of prostate biopsy—a TRUS (trans-rectal ultrasound) guided or transrectal biopsy, and a template (transperineal) biopsy. TRUS biopsy involves insertion of an ultrasound probe into the rectum and scanning the prostate in order to guide where to extract the cells from. Normally 10 to 12 small pieces of tissue are taken from different areas of the prostate.

A template biopsy involves inserting the biopsy needle into the prostate through the skin between the testicles and the rectum (the perineum). The needle is inserted through a grid (template). A template biopsy takes more tissue samples from more areas of the prostate than a TRUS biopsy. The number of samples taken will vary but can be around 20 to 50 from different areas of the prostate.

Prostate Cancer Treatment

Patients with metastatic disease are primarily treated with hormone deprivation therapy. However, the cancer invariably becomes resistant to treatment leading to disease progression and eventually death. Treatment of patients with metastatic prostate cancer is clinically very challenging for a number of reasons, which include: i) the variability in patient response to hormone treatment (i.e. time prior to relapse and becoming castrate resistant), ii) the detrimental effects of hormone manipulation therapy on patients and iii) the myriad new treatment options available for castrate resistant patients. In some cases, treatment of prostate cancer can be placing the patient under active surveillance.

The response to hormone manipulation/ablation therapy is highly variable. Some men fail to respond to treatment while others relapse early (i.e. within 6 months), the majority relapse within 18 months (late relapse) and the rest respond well to the treatment often taking several years before relapsing (delayed relapse). Early identification of patients who will have a poor response will provide a clinical opportunity to offer them a different treatment approach that may perhaps improve their prognosis. However, there is no means currently to identify such patients except for when they exhibit biochemical progression with rising serum PSA, or become clinically symptomatic, in which case they get offered a different treatment strategy. This regime however goes hand in hand with a number of detrimental effects such as bone loss, increased obesity, decreased insulin sensitivity increasing the incidence of diabetes, adversely altered lipid profiles leading to cardiovascular disease and an increased rate of heart attacks. For these reasons offering hormone manipulation requires a lot of clinical consideration particularly as most of the patients requiring such treatment are elderly patients and such treatment could overall be detrimental rather than beneficial.

Due to ever-emerging new treatments or second line therapies for patients with advanced metastatic cancer in the past decade, the treatment of men with castrate resistant prostate cancer is dramatically changing. Prior to 2004, the only treatment option for these patients was medical or surgical castration then palliation. Since then several chemotherapy treatments have emerged starting with docetaxel, which has shown to improve survival for some patients. This was followed by five additional agents (FDA-approved) including new hormonal agents targeting the androgen receptor (AR) such as the AR antagonist Enzalutamide, agents to inhibit androgen biosynthesis such as Abiraterone, two agents designed specifically to affect the androgen axis, sipuleucel-T, which stimulates the immune system, cabazitaxel chemotherapeutic agent and radium-223, a radionuclide therapy. Other treatments include targeted therapies such as the P13K inhibitor BKM120 and an Akt inhibitor AZD5363. Therefore, it is crucially important to be able to identify patients that would benefit from these treatments and those that will not. Identification of prognostic indicators capable of predicting response to hormone manipulation and to the above list of alternative treatments is very important and would have great clinical impact in managing these patients. In addition, the only current clinically available means to diagnose metastasis is by imaging. Markers that are being put forward include circulating tumour cells and urine bone degradation markers. A test for metastasis per se could radically alter patient treatment. The data presented here in suggest that extracellular vesicle RNA may have the potential to overcome these issues, particularly as studies have shown a role for EVs such as exosomes in aiding metastasis. A test for metastasis per se could radically alter patient treatment.

Prostate cancer can be scored using the Gleason grading system, which uses a histological analysis to grade the progression of the disease. A grade of 1 to 5 is assigned to the cells under examination, and the two most common grades are added together to provide the overall Gleason score. Grade 1 closely resembles healthy tissue, including closely packed, well-formed glands, whereas grade 5 does not have any (or very few) recognisable glands. Gleason scores of less than 6 have a good prognosis, whereas scores of 6 or more are classified as more aggressive. The Gleason score was refined in 2005 by the International Society of Urological Pathology and references herein refer to these scoring criteria [49]. The Gleason score is detected in a biopsy, i.e. in the part of the tumour that has been sampled. A Gleason 6 prostate may have small foci of aggressive tumour that have not been sampled by the biopsy and therefore the Gleason is a guide. The lower the Gleason score the smaller the proportion of the patients will have aggressive cancer. Gleason score in a patient with prostate cancer can go down to 2, and up to 10. Because of the small proportion of low Gleasons that have aggressive cancer, the average survival is high, and average survival decreases as Gleason increases due to being reduced by those patients with aggressive cancer (i.e. there is a mixture of survival rates at each Gleason score).

Prostate cancers can be staged according to how advanced they are. This is based on the TMN scoring as well as any other factors, such as the Gleason score and/or the PSA test. The staging can be defined as follows:

Stage I:

T1, N0, M0, Gleason score 6 or less, PSA less than 10

OR

T2a, N0, M0, Gleason score 6 or less, PSA less than 10

Stage IIA:

T1, N0, M0, Gleason score of 7, PSA less than 20

OR

T1, N0, M0, Gleason score of 6 or less, PSA at least 10 but less than 20:

OR

T2a or T2b, N0, M0, Gleason score of 7 or less, PSA less than 20

Stage IIB:

T2c, N0, M0, any Gleason score, any PSA

OR

T1 or T2, N0, M0, any Gleason score, PSA of 20 or more:

OR

T1 or T2, N0, M0, Gleason score of 8 or higher, any PSA

Stage III:

T3, N0, M0, any Gleason score, any PSA

Stage IV:

T4, N0, M0, any Gleason score, any PSA

OR

Any T, N1, MO, any Gleason score, any PSA:

OR

Any T, any N, M1, any Gleason score, any PSA

In the present invention, an aggressive cancer is defined functionally or clinically: namely a cancer that can progress. This can be measured by PSA failure. When a patient has surgery or radiation therapy, the prostate cells are killed or removed. Since PSA is only made by prostate cells the PSA level in the patient's blood reduces to a very low or undetectable amount. If the cancer starts to recur, the PSA level increases and becomes detectable again. This is referred to as “PSA failure”. An alternative measure is the presence of metastases or death as endpoints.

Prostate cancer can be scored using the Prostate Imaging Reporting and Data System (PI-RADS) grading system designed to standardise non-invasive MRI and related image acquisition and reporting, potentially useful in the initial assessment of the risk of clinically significant prostate cancer. A PI-RADS score is given according to each variable parameter. The scale is based on a score “Yes” or “No” for Dynamic Contrast-Enhanced (DCE) parameter, and from 1 to 5 for T2-weighted (T2W) and Diffusion-weighted imaging (DWI). The score is given for each lesion, with 1 being most probably benign and 5 being highly suspicious of malignancy:

PI-RADS 1: very low (clinically significant cancer is highly unlikely to be present)

PI-RADS 2: low (clinically significant cancer is unlikely to be present)

PI-RADS 3: intermediate (the presence of clinically significant cancer is equivocal)

PI-RADS 4: high (clinically significant cancer is likely to be present)

PI-RADS 5: very high (clinically significant cancer is highly likely to be present)

Increase in Gleason score, stage as defined above or PI-RADS grade can also be considered as progression. However, a PUR signature characterisation is independent of Gleason, stage, PI-RADS and PSA. It provides additional information about the development of aggressive cancer in addition to Gleason, stage, PI-RADS and PSA. It is therefore a useful independent predictor of outcome. Nevertheless, PUR signature status can be combined with Gleason, tumour stage, PI-RADS score and/or PSA.

In some methods of the invention the PUR signatures can be used alongside MRI to aid decision making on whether to biopsy or not, particularly in men with PI-RADS 3 and 4. PUR could also be used to confirm the absence of clinically significant prostate cancer in men with PI-RADS 1 and 2.

Thus, the methods of the invention provide methods of classifying cancer, some methods comprising determining the expression status or expression status of a one or more members of a biomarker panel. The expression of the panel of genes may be determined using a method of the invention.

By “clinical outcome” it is meant that for each patient whether the cancer has progressed. For example, as part of an initial assessment, those patients may have prostate specific antigen (PSA) levels monitored. When it rises above a specific level, this is indicative of relapse and hence disease progression. Histopathological diagnosis may also be used. Spread to lymph nodes, and metastasis can also be used, as well as death of the patient from the cancer (or simply death of the patient in general) to define the clinical endpoint. Gleason scoring, cancer staging and multiple biopsies (such as those obtained using a coring method involving hollow needles to obtain samples) can be used. Clinical outcomes may also be assessed after treatment for prostate cancer. This is what happens to the patient in the long term. Usually the patient will be treated radically (prostatectomy, radiotherapy) to effectively remove or kill the prostate. The presence of a relapse or a subsequent rise in PSA levels (known as PSA failure) is indicative of progressed cancer. The PUR signature cancer populations identified using methods of the invention comprise subpopulations of cancers that may progress more quickly.

Accordingly, any of the methods of the invention may be carried out in patients in whom prostate cancer is suspected. Importantly, the present invention allows a prediction of cancer progression before treatment of cancer is provided. This is particularly important for prostate cancer, since many patients will undergo unnecessary treatment for prostate cancer when the cancer would not have progressed even without treatment.

In some methods of the invention, the PUR signature calculated from the expression status or expression status of a one or more genes can be combined with the results of MRI imaging diagnostics to provide an improved diagnosis or prognosis of prostate cancer. In some methods of the invention, the PUR signature calculated from the expression status or expression status of a one or more genes can be combined with multiple imaging techniques, or combined imaging scores (such as PI-RADS as described above) to provide an improved diagnosis or prognosis of prostate cancer.

Determining the expression status of a gene may comprise determining the expression status of the gene. Expression status and levels of expression as used herein can be determined by methods known to the skilled person. For example, this may refer to the up or down-regulation of a particular gene or genes, as determined by methods known to a skilled person. Epigenetic modifications may be used as an indicator of expression, for example determining DNA methylation status, or other epigenetic changes such as histone marking, RNA changes or conformation changes. Epigenetic modifications regulate expression of genes in DNA and can influence efficacy of medical treatments among patients. Aberrant epigenetic changes are associated with many diseases such as, for example, cancer. DNA methylation in animals influences dosage compensation, imprinting, and genome stability and development. Methods of determining DNA methylation are known to the skilled person (for example methylation-specific PCR, matrix-assisted laser desorption/ionisation time-of-flight mass spectrometry, use of microarrays, reduced representation bisulfate sequencing (RRBS) or whole genome shotgun bisulfate sequencing (WGBS). In addition, epigenetic changes may include changes in conformation of chromatin.

The expression status of a gene may also be judged examining epigenetic features. Modification of cytosine in DNA by, for example, methylation can be associated with alterations in gene expression. Other way of assessing epigenetic changes include examination of histone modifications (marking) and associated genes, examination of non-coding RNAs and analysis of chromatin conformation. Examples of technologies that can be used to examine epigenetic status are provided in the references [50,51,52,53,54]

Proteins can also be used to determine expression status, and suitable method to determine expressed protein levels are known to the skilled person.

The present invention shall now be further described with reference to the following examples, which are present for the purposes of illustration only and are not to be construed as being limiting on the invention.

### EXAMPLES

### Example 1

### Patient Samples and Clinical Criteria

First-catch urine samples collected with a digital rectal examination (DRE) were collected at diagnosis between 2009 and 2015 from clinics at the Norfolk and Norwich University Hospital (NNUH, Norwich, UK), Royal Marsden Hospital (RMH, London, UK), St. James Hospital (Dublin, Republic of Ireland) and from primary care and urology clinics of Emory Healthcare (Atlanta, USA). Active surveillance eligibility criteria can include the following: histologically proven prostate cancer, age 50-80, clinical stage T1/T2, PSA <15 ng/ml, Gs ≤6 (Gs ≤3+4 if age >65), and <50% percent positive biopsy cores. Disease progression criteria were either: PSA velocity >1 ng/ml per year or adverse histology on repeat biopsy, defined as primary Gs ≥4+3 or ≥50% cores positive for cancer. Criteria for MP-MRI progression were either: detection of >1 cm3 prostate tumour, an increase in volume >100% for lesions between 0.5-1 cc, or T3/4 disease.

D'Amico classification used Gleason and PSA criteria as described in reference 2. CAPRA classification used the criteria as described in reference 8. Sample collections were ethically approved in their country of origin. Trans-rectal ultrasound (TRUS) guided biopsy was used to provide biopsy information. Men were defined to have no evidence of cancer (NEC) with a PSA normal for their age or lower [55] and as such, were not subjected to biopsy. Men with a PSA >100 ng/mL were determined to have metastatic disease and were excluded from analyses.

### Example 2

### Sample Processing

Briefly, urine was centrifuged (1200 g 10 min, 6° C.) within 30 min of collection to pellet cellular material. Supernatant extracellular vesicles (EVs) were then harvested by microfiltration as described in reference 56 and RNA extracted (RNeasy micro kit, #74004, Qiagen). RNA was amplified as cDNA with an Ovation PicoSL WTA system V2 (Nugen #3312-48). 5-20 ng of total RNA was amplified where possible, down to 1 ng input in 10 samples. cDNA yields were mean 3.83 μg (1-6 μg).

DRE-Urine Collection for DNA/RNA
1. Prepare 30 ml Universal collection bottles, one per patient. Label the collection bottle with patient number, patient name and date.
2. Obtain consent from the patient. Before sample collection the clinician should perform a DRE on the patient's prostate as follows: Apply pressure on the prostate, enough to depress the entire surface of the prostate approximately 1 cm, from the base to the apex and from lateral to the median line for each lobe. Perform exactly 3 strokes for each lobe.
3. Ask the patient to provide ‘first catch’ urine (the first ˜30 ml passed) in the Universal sample tube.
4. Place the sample in a Styrofoam box with ice packs in the clinic room. (can use ice, but not ice/water mix as this cools the sample too much causing the urine to go cloudy).
5. Maintain on ice. Proceed to section 4 as soon as possible—within 15 min is best for optimal RNA yields. If this is not possible then within 4 hr. Note the time between sample collection and processing.
   Within 15 min of sample collection:
6. Invert the DRE urine sample 4 times to resuspend any sediment.
7. Aliquot 4.5 ml of whole urine into capped tubes (3×1 ml, 3×0.5 ml) and freeze at
8. −80° C. (or place on dry ice and transfer to the −80° C. later).
9. If the total volume of the urine is less than 15 ml then only freeze 3×0.5 ml.
10. Proceed immediately to cell sedimentation.
11. If this is not possible and the urine is to be frozen at −80° C. for processing the next day (or later) then first add EDTA to 40 mM (2 ml of 500 mM EDTA for 25 ml urine).

Urine Sample Processing
1. Harvest the cells by centrifugation at 1200 g for 5 min at 6° C. **Ensure that the centrifuge brake speed is set on a slow deceleration setting to avoid disruption of pellet and loss of sample.
2. Carefully and slowly pipette off the supernatant into the ‘EV’ 30 ml Universal tube. Place on crushed ice until ready to extract EV RNA.
2. Record the details of cell pellet size and appearance (e.g. large white, small, barely visible, clear/cloudy/yellow/red) and place immediately on dry ice to snap freeze the cell pellet.
3. Pause Point: Maintain the cell pellets on dry ice and the urine supernatants on normal ice while you are waiting for the other samples from the clinic to arrive. Then, either:
a) Same day extraction: Proceed to Cell DNA/RNA extraction in the afternoon, or
ii) Next Day extraction: Transfer the cell pellet on dry ice to a −80° C. freezer for DNA/RNA extraction the next day, or
iii) Later extraction: Make up the volume of the cell pellet to ˜1 ml in PBS and freeze on dry ice. Transfer to −80° C. freezer for subsequent extraction.

DNA and RNA Extraction from Cells
1. Place the cell pellets on wet ice.
2. While still frozen, add 600 μl of RLT PLUS buffer (with DTT added)
3. The sample will thaw rapidly in the RLT PLUS lysis buffer, as soon as it is fully defrosted, mix the sample by pipetting or vortexing and then load onto a QIAshredder column and centrifuge at 12,000 g for 2 min (or pass the sample/lysis buffer through a 20 gauge sterile syringe and needle (0.9mm) 10-15 times).
4. Pipette the QIAshredder supernatant (taking care not to disturb any pellet that may have formed) onto the AllPrep DNA column provided in the kit.
5. Centrifuge the AllPrep DNA column at 10,000 g for 30 sec, the flow through contains the RNA for extraction; transfer the flow through to a pre-labelled 2 ml non-stick tube.
6. Transfer the DNA column to a new collection tube and place at 4° C. until RNA extraction is completed.
7. Measure the volume of the RNA flow through from step 5, and add an equal volume of 70% ethanol.
8. Mix by pipetting and proceed immediately to RNA harvest.

RNA Harvest from Cell Pellet
1. Pipette 750 μl of the sample/ethanol onto an RNeasy spin column (supplied in the kit), spin full speed ˜10 sec in a microfuge. Discard flow through.
2. Repeat until the entire sample has been run through the column.
3. Wash the column with 350 μl of ‘RW1 Buffer’.
4. For each column mix 10 μl of ‘DNase I’ stock solution to 70 μl of ‘Buffer RDD’. Mix by inversion. Add the 80 μl mix directly to the membrane of each ‘Mini Elute Columns’). Leave at room temperature for 15 min.
5. Add 350 μl RW1, spin 15 sec, discard flow through.
6. Add 500 μl RPE and spin max speed 15 sec.
7. Discard flow through and ‘collection tube’.
8. Place the RNeasy spin column in new collection tube.
9. Centrifuge with the tube lid open at max speed for 2 min.
10. Discard flow through and ‘collection tube’.
11. Place the RNeasy spin column in a 1.5 ml non-stick tube containing 1 ul of 1 μg/ul glycogen in 2× TE.
12. Add 30 μl of nuclease free water (provided in the kit) to the centre of the membrane.
13. Let sit for 2-3 min, then centrifuge at max speed for 1 min.
14. Transport the RNA samples on ice to the −80° C. freezer.

EV RNA Harvest and Extraction

EVs were harvested by ultracentrifugation described in reference 56.

EV Harvest by 100 kDa Filter Centrifugation:

Process the urine supernatant from as follows:

If the urine supernatant has been stored frozen (−80° C.) then thaw in cold water, and then vortex for 90 sec before continuing.

For each sample, label the following with the sample number and an ‘X’ for EV:
a) 30 ml Syringe
b) Amicon UltraCel-100k Centrifugal filter unit (UFC910096) or (#UFC910096, Millipore)
c) 1.5 ml non-stick tube (Ambion AM12450)
d) 30 ml Universal tube

NB: Add 40 μl of 1M DTT per ml RLT buffer (Qiagen RNeasy Micro kit). DTT-RLT can be stored at room temperature for up to one month).
1. Spin the supernatant at 2000 g 5 min r/t.
2. Filter the urine sample: Pull the plunger out of a 30 ml syringe and insert the barrel into a 0.8 μm filter. Pour the urine into the syringe. Insert the plunger and push the urine into the UltraCel 100k spin filter unit.
3. If the urine volume is >15 ml then lay the syringe (containing remaining urine) horizontally onto on a drip tray lined with clean paper towel.
4. Spin the UltraCel 100k unit at 3,400 g 10 min 21° C.
5. If the urine will not pass through the filter then use a 1 ml pipette to squirt the filter surfaces with the urine and re-spin 5 min. Repeat until the urine volume is reduced to <500 μl. Take care not to touch or damage the filters themselves.
6. Remove the UltraCel 100k unit from the centrifuge and discard flow through. Add the rest of the urine sample from the syringe/filter to the spin unit.
7. Spin the UltraCel 100k unit at 3,400 g 10 min 21° C. until the volume of the sample has reduced to <500 μl.
8. Add 15 ml of PBS.
9. Spin at 3,400 g 10 min 21° C. or until the volume is ˜200 μl.
10. Discard flow through.
11. Pipette out the concentrated sample using a 200 μl pipette. Transfer to a 1.5 ml non-stick tube. Measure the volume (Should be 200 μl in total). If less, then make up the volume to 200 μl with PBS.
12. Immediately rinse the filter unit with 700 μl of RLT/DTT buffer from the Qiagen Micro RNeasy kit and add this to the sample tube.
13. Add ethanol to a final concentration of 35%.
To do this, measure the total vol (ie Sample+RLT). Then multiply this by 0.54 and add this amount of 100% ethanol (usually ˜485 μl ethanol).
14. Vortex 10-20 sec to mix and disrupt the microvesicles.
15. Proceed directly to section 6.2 for optimal quality RNA, or freeze AT −20 or −80° C. overnight for extraction the next day (RNA yield and quality will be of lower).

RNA Extraction from EVs Using a Qiagen RNeasy Micro kit.

Preparation:
a) Transfer one RNeasy MiniElute spin column per sample from the fridge the night before and leave at room temperature.
b) If frozen, warm the samples to room temperature before applying to column.
c) Warm the elution water to 45° C.

For each sample you will need:
a) An RNeasy MiniElute spin column placed in a 7.5 ml Bijou tube.
b) A 1.5 ml non-stick tube with sample number, date and X (for EV) containing 1 μl of 1 μg/μl glycogen.
c) 80 μl of DNAse 1 mix (10 μl of ‘DNase I’ stock solution with 70 μl of ‘Buffer RDD’. Mix by inversion). Procedure
   1. Place a RNeasy MiniElute spin column in the neck of a 7□5 ml Bijou collection tube and place that into a large centrifuge—set at 21° C. 1500 g.
   2. Load half of the sample (˜700 μl) onto the micro filter cartridge.
   3. Spin 10-15 sec (or until the mix has passed through the filter—can be up to 1 min—the samples that don't spin through the 100 kDa unit can cause blockage on the Qiagen column and need longer spinning).
   4. Repeat steps 2) and 3).
   5. 350 μl of ‘RW1 Buffer’ wash, Spin 1500 g 10-15 sec.
   6. Add 80 μl DNAse 1 mix (see above) directly to the membrane of each ‘Mini Elute Column’). Leave at room temperature in the centrifuge for 15 min.—can empty the Bijou collection tubes at this point if necessary.
   7. 350 μl RW1, spin 15 sec.
   8. 500 μl RPE, spin 15 sec.
   9. 500 μl of freshly diluted 80% ethanol (use the RNAse-free H2O in the kit) to each ‘Mini Elute Column’. Spin 2000 g 2 min.
   10. Transfer the ‘Mini Elute Columns’ into new Qiagen collection tubes and place in a microcentrifuge, Spin with the tube lid open at max speed for 5 min. Make sure the tube lid is open as this will aid drying of the filter. Discard flow through and ‘collection tube’.
   11. Place the ‘Mini Elute Column’ into a labelled 1.5 ml Ambion non-stick collection tube containing 1 ul of 1 ug/ul glycogen in 2× TE. Add 20 μl of 45° C. Qiagen nuclease free water (provided in the kit) to the centre of the membrane.
   12. Wait 2-3 min and then centrifuge at max speed for 1 min.
   13. Store the RNA sample in a −80° C. freezer.

Notes: Air drying sample columns for 5 min prior to adding elution water is essential.

Warming elution water to 45° C. can increase yield.

Extra washes with RLT, RW1 and RPE help decrease false 230 and 280 nm Spectrophotometer peaks.

Mixing RPE stock with Ethanol on a daily basis helps with long term consistency of yield.

Amplification of RNA

Amplify 15-20 ng of EV RNA as quantified by Bioanalyzer.

Use the Nugen Ovation 2 RNA amplification kit as manufacturer's instructions (Nugen Ovation PicoSL WTA2 (3312-48)).

Clean up the Amplification products

QIAGEN MinElute Reaction Cleanup Kit (Cat. no. 28204).
1. Aliquot 300 μl of Buffer ERC into a labeled 1.5 ml microcentrifuge tube
2. Add the entire volume (40 μl) of the Nugen Ovation SPIA reaction to the tube.
3. Vortex for 5 sec, then spin briefly (5 sec) in a microcentrifuge.
4. Label a MinElute spin column and place in a collection tube.
5. Load the sample/buffer mixture onto the column.
6. Centrifuge for 1 min at 13,000 g in a microcentrifuge.
7. Discard the flow-through and replace the column in the same collection tube.
8. Add 750 μl of Buffer PE to the column.
9. Centrifuge for 1 min at maximum speed.
10. Discard the flow-through and replace the column in the same collection tube.
11. Centrifuge the column for an additional 2 min at maximum speed to remove all residual Buffer PE. Note: Residual ethanol from the wash buffer will not be completely removed unless the flow-through is discarded before this additional centrifugation.
12. Discard the flow-through with the collection tube. Blot the column onto clean, absorbent paper to remove any residual wash buffer from the tip of the column. Note: Blotting the column tip prior to transferring it to a clean tube is necessary to prevent any wash buffer transferring to the eluted sample.
13. Place the column into a clean, labelled 1.5 ml microcentrifuge tube.
14. Add 20 μl of room temperature, Nuclease-free Water (green: D1) from the NuGEN® kit to the centre of each column. Note: Ensure that the water is dispensed directly onto the membrane for complete elution of the bound cDNA.
15. Let the column stand for 1 min at room temperature.
16. Centrifuge for 1 min at maximum speed.
17. Discard the column and measure the volume recovered.
18. Mix the sample by vortexing, then spin briefly.
19. Add 1/10th vol of 1× TE and store at −80° C.

### Example 3

### Expression Analyses

NanoString® expression analysis (167 probes, 164 genes, Table 2) of 100 ng cDNA was performed at the Human Dendritic Cell Laboratory, Newcastle University, UK. 137 probes were selected based on previously proposed controls plus prostate cancer diagnostic and prognostic biomarkers within tissue and control probes. 30 additional probes were selected as overexpressed in prostate cancer samples when next generation sequence data generated from 20 urine EV RNA samples were analysed. Target gene sequences were provided to NanoString®, who designed the probes according to their protocols [57]. Data were adjusted relative to internal positive control probes as stated in NanoString®'s protocols. The ComBat algorithm was used to adjust for inter-batch and inter-cohort bias [58].

TABLE 2
Genes initially identified for analysis with NanoString ® microarrays

| Gene | Full name | Accession number |
|---|---|---|
| AATF | apoptosis antagonizing transcription factor | NM_012138.3 |
| ABCB9 | ATP binding cassette subfamily B member 9 | NM_001243013.1 |
| ACTR5 | ARP5 actin-related protein 5 homolog | NM_024855.3 |
| AGR2 | anterior gradient 2, protein disulphide isomerase | NM_006408.2 |
| | family member | |
| ALAS1 | 5′-aminolevulinate synthase 1 | NM_000688.4 |
| AMACR | alpha-methylacyl-CoA racemase | NM_014324.4 |
| AMH | anti-Mullerian hormone | NM_000479.3 |
| ANKRD34B | ankyrin repeat domain 34B | NM_001004441.2 |
| ANPEP | alanyl aminopeptidase, membrane | NM_001150.1 |
| APOC1 | apolipoprotein C1 | NM_001645.3 |
| AR ex 9 | Androgen Receptor splice variant | ENST00000514029.1 |
| AR ex 4-8 | Androgen Receptor | NM_000044.2 |
| ARHGEF25 | Rho guanine nucleotide exchange factor 25 | NM_001111270.2 |
| AURKA | aurora kinase A | NM_003600.2 |
| B2M | beta-2-microglobulin | NM_004048.2 |
| B4GALNT4 | beta-1,4-N-acetyl-galactosaminyltransferase 4 | NM_178537.4 |
| BRAF | B-Raf proto-oncogene, serine/threonine kinase | NM_004333.3 |
| BTG2 | BTG anti-proliferation factor 2 | NM_006763.2 |
| CACNA1D | calcium voltage-gated channel subunit alphal D | NM_000720.3 |
| CADPS | calcium dependent secretion activator | NM_183394.2 |
| CAMK2N2 | calcium/calmodulin dependent protein kinase II | NM_033259.2 |
| | inhibitor 2 | |
| CAMKK2 | calcium/calmodulin dependent protein kinase kinase 2 | NM_006549.3 |
| CASKIN1 | CASK interacting protein 1 | NM_020764.3 |
| CCDC88B | coiled-coil domain containing 88B | NM_032251.5 |
| CDC20 | cell division cycle 20 | NM_001255.2 |
| CDC37L1 | cell division cycle 37 like 1 | NM_017913.2 |
| CDKN3 | cyclin dependent kinase inhibitor 3 | NM_005192.3 |
| CERS1 | ceramide synthase 1 | NM_198207.2 |
| CKAP2L | cytoskeleton associated protein 2 like | NM_152515.3 |
| CLIC2 | chloride intracellular channel 2 | NM_001289.4 |
| CLU | clusterin | NM_203339.1 |
| COL10A1 | collagen type X alpha 1 chain | NM_000493.3 |
| COL9A2 | collagen type IX alpha 2 chain | NM_001852.3 |
| CP | ceruloplasmin | NM_000096.3 |
| MIATNB | MIAT neighbour | CTA_211A95.1 |
| DLX1 | distal-less homeobox 1 | NM_001038493.1 |
| DNAH5 | dynein axonemal heavy chain 5 | NM_001369.2 |
| DPP4 | dipeptidyl peptidase 4 | NM_001935.3 |
| ECI2 | enoyl-CoA delta isomerase 2 | NM_006117.2 |
| EIF2D | eukaryotic translation initiation factor 20 | NM_006893.2 |
| EN2 | engrailed homeobox 2 | NM_001427.3 |
| TMPRSS2/ERG | transmembrane protease, serine 2/ERG fusion | Fusion_0120.1 |
| | | EU432099.1 |
| ERG | ERG, ETS transcription factor | NM_001243428.1 |
| ERG 3 ex 4-5 | ERG, ETS transcription factor | NM_004449.4 |
| ERG3 ex 6-7 | ERG, ETS transcription factor | NM_182918.3 |
| FOPS | farnesyl diphosphate synthase | NM_001135822.1 |
| FOLH1 | folate hydrolase 1 | NM_004476.1 |
| GABARAPL2 | GABA type A receptor associated protein like 2 | NM_007285.6 |
| GAPDH | glyceraldehyde-3-phosphate dehydrogenase | NM_002046.3 |
| GCNT1 | glucosaminyl (N-acetyl) transferase 1, core 2 | NM_001097633.1 |
| GDF15 | growth differentiation factor 15 | NM_004864.2 |
| GJB1 | gap junction protein beta 1 | NM_000166.5 |
| GOLM1 | golgi membrane protein 1 | NM_016548.3 |
| HIST1H1C | histone cluster 1 H1 family member c | NM_005319.3 |
| HIST1H1 E | histone cluster 1 H1 family member e | NM_005321.2 |
| HIST1H2BF | histone cluster 1 H2B family member f | NM_003522.3 |
| HIST1H2BG | histone cluster 1 H2B family member g | NM_003518.3 |
| HIST3H2A | histone cluster 3 H2A | NM_033445.2 |
| HMBS | hydroxymethylbilane synthase | NM_000190.3 |
| HOXC4 | homeobox C4 | NM_014620.4 |
| HOXC6 | homeobox C6 | NM_153693.3 |
| HPN | hepsin | NM_182983.1 |
| HPRT1 | hypoxanthine phosphoribosyltransferase 1 | NM_000194.1 |
| IF157 | intraflagellar transport 57 | NM_018010.2 |
| IGFBP3 | insulin like growth factor binding protein 3 | NM_000598.4 |
| IMPDH2 | inosine monophosphate dehydrogenase 2 | NM_000884.2 |
| ISX | intestine specific homeobox | NM_001008494.1 |
| ITGBL1 | integrin subunit beta like 1 | NM_004791.2 |
| ITPR1 | inositol 1,4,5-trisphosphate receptor type 1 | NM_001099952.1 |
| KLK2 | kallikrein related peptidase 2 | NM_005551.3 |
| KLK3 ex 1-2 | kallikrein related peptidase 3 | NM_001030048.1 |
| KLK3 ex 2-3 | kallikrein related peptidase 3 | NM_001648.2 |
| KLK4 | kallikrein related peptidase 4 | NM_004917.3 |
| LBH | limb bud and heart development | NM_030915.3 |
| POTEH-AS1 | POTEH antisense RNA 1 (POTEH-AS1), long non- | NR_110505.1 |
| | coding RNA. prostate-specific P712P mRNA | |
| MAK | male germ cell associated kinase | NM_005906.3 |
| MAPK8IP2 | mitogen-activated protein kinase 8 interacting protein | NM_012324.2 |
| | 2 | |
| MARCH5 | membrane associated ring-CH-type finger 5 | NM_017824.4 |
| MCM7 | minichromosome maintenance complex component 7 | NM_182776.1 |
| MCTP1 | multiple C2 and transmembrane domain containing 1 | NM_024717.4 |
| MDK | midkine (neurite growth-promoting factor 2) | NM_001012334.1 |
| MED4 | mediator complex subunit 4 | NM_001270629.1 |
| MEMO1 | mediator of cell motility 1 | NM_001137602.1 |
| MET | MET proto-oncogene, receptor tyrosine kinase | NM_001127500.1 |
| MEX3A | mex-3 RNA binding family member A | NM_001093725.1 |
| MFSD2A | major facilitator superfamily domain containing 2A | NM_032793.4 |
| MGAT5B | mannosyl (alpha-1,6-)-glycoprotein beta-1,6-N-acetyl- | NM_144677.2 |
| | glucosaminyltransferase, isozyme B | |
| MIR146A | microRNA 146a | ENST00000517927.1 |
| MIR4435-2HG | MIR4435-2 host gene | ENST00000409569b.1 |
| MKI67 | marker of proliferation Ki-67 | NM_002417.2 |
| MME | membrane metalloendopeptidase | NM_000902.2 |
| MMP11 | matrix metallopeptidase 11 | NM_005940.3 |
| MMP25 | matrix metallopeptidase 25 | NM_022468.4 |
| MMP26 | matrix metallopeptidase 26 | NM_021801.3 |
| MNX1 | motor neuron and pancreas homeobox 1 | NM_005515.3 |
| MSMB | microseminoprotein beta | NM_002443.2 |
| MXI1 | MAX interactor 1, dimerization protein | NM_001008541.1 |
| MYOF | myoferlin | NM_013451.3 |
| NAALADL2 | N-acetylated alpha-linked acidic dipeptidase like 2 | NM_207015.2 |
| NEAT1 | nuclear paraspeckle assembly transcript 1 (non- | NR_028272.1 |
| | protein coding) | |
| NKAIN1 | Na+/K+ transporting ATPase interacting 1 | NM_024522.2 |
| NLRP3 | NLR family pyrin domain containing 3 | NM_001079821.2 |
| OGT | O-linked N-acetylglucosamine (GlcNAc) transferase | NM_181672.1 |
| OR51E2 | olfactory receptor family 51 subfamily E member 2 | NM_030774.2 |
| PALM3 | paralemmin 3 | NM_001145028.1 |
| PCA3 | prostate cancer associated 3 (non-protein coding) | NR_015342.1 |
| PCSK6 | proprotein convertase subtilisin/kexin type 6 | NM_138320.1 |
| PDLIM5 | PDZ and LIM domain 5 | NR_046186.1 |
| PLPP1 | phospholipid phosphatase 1 | NM_176895.1 |
| PPFIA2 | PTPRF interacting protein alpha 2 | NM_003625.2 |
| PPP1R12B | protein phosphatase 1 regulatory subunit 12B | NM_001167857.1 |
| PSTPIP1 | proline-serine-threonine phosphatase interacting | XM_006720737.1 |
| | protein 1 | |
| PIN | pleiotrophin | NM_002825.5 |
| PTPRC | protein tyrosine phosphatase, receptor type C | NM_080923.2 |
| PVT1 | Pvt1 oncogene (non-protein coding) | NR_003367.2 |
| RAB17 | RAB17, member RAS oncogene family | NR_033308.1 |
| RIOK3 | RIO kinase 3 | NM_003831.3 |
| RN F157 | ring finger protein 157 | NM_052916.2 |
| MRPL46 | mitochondrial ribosomal protein L46 | ENST00000561140.1 |
| RPL18A | ribosomal protein L18a | NM_000980.3 |
| RPL23AP53 | ribosomal protein L23a pseudogene 53 | NR_003572.2 |
| RPLP2 | ribosomal protein lateral stalk subunit P2 | NM_001004.3 |
| RPS10 | ribosomal protein S10 | NM_001014.3 |
| RPS11 | ribosomal protein S11 | NM_001015.3 |
| SACM1L | SAC1 suppressor of actin mutations 1-like (yeast) | NM_014016.3 |
| SCHLAP1 | SWI/SNF complex antagonist associated with | NR_104320.1 |
| | prostate cancer 1 (non-protein coding) | |
| SEC61A1 | Sec61 translocon alpha 1 subunit | NM_013336.3 |
| SERPINB5 | serpin family B member 5 | NM_002639.4 |
| SFRP4 | secreted frizzled related protein 4 | NM_003014.2 |
| SIM2 | single-minded family bHLH transcription factor 2 | NM_005069.3 |
| SIM2 | single-minded family bHLH transcription factor 2 | NM_009586.3 |
| SIRT1 | sirtuin 1 | NM_012238.4 |
| SLC12A1 | solute carrier family 12 member 1 | NM_000338.2 |
| SLC43A1 | solute carrier family 43 member 1 | NM_003627.5 |
| SLC4A1 | solute carrier family 4 member 1 | NM_000342.3 |
| SMAP1 | small ArfGAP 1 | NM_021940.3 |
| SMIM1 | small integral membrane protein 1 (Vel blood group) | ENST00000444870.1 |
| SNCA | synuclein alpha | NM_007308.2 |
| SNORA20 | Small nucleolar RNA SNORA20 | NR_002960.1 |
| SPINK1 | serine peptidase inhibitor, Kazal type 1 | NM_003122.2 |
| SPON2 | spondin 2 | NM_012445.1 |
| SRSF3 | serine and arginine rich splicing factor 3 | NM_003017.4 |
| SSPO | SCO-spondin | NM_198455.2 |
| SSTR1 | somatostatin receptor 1 | NM_001049.2 |
| ST6GALNAC1 | ST6 N-acetylgalactosaminide alpha-2,6- | ENST00000592042.1 |
| | sialyltransferase 1 | |
| STEAP2 | STEAP2 metalloreductase | NM_152999.2 |
| STEAP4 | STEAP4 metalloreductase | NM_024636.2 |
| STOM | stomatin | NM_004099.5 |
| SULF2 | sulfatase 2 | NM_001161841.1 |
| SULT1A1 | sulfotransferase family 1A member 1 | NM_177534.2 |
| SYNM | synemin | NM_015286.4 |
| TBP | TATA-box binding protein | NM_001172085.1 |
| TDRD1 | Tudor domain containing 1 | NM_198795.1 |
| TERF2IP | TERF2 interacting protein | NM_018975.3 |
| TERT | telomerase reverse transcriptase | NM_198253.1 |
| TFDP1 | transcription factor Dp-1 | NM_007111.4 |
| TIMP4 | TIMP metallopeptidase inhibitor 4 | NM_003256.2 |
| TMCC2 | transmembrane and coiled-coil domain family 2 | NM_014858.3 |
| TMEM45B | transmembrane protein 45B | NM_138788.3 |
| TMEM47 | transmembrane protein 47 | NM_031442.3 |
| TMEM86A | transmembrane protein 86A | NM_153347.1 |
| TRPM4 | transient receptor potential cation channel subfamily | NM_001195227.1 |
| | M member 4 | |
| TWIST1 | twist family bHLH transcription factor 1 | NM_000474.3 |
| UPK2 | uroplakin 2 | NM_006760.3 |
| VAX2 | ventral anterior homeobox 2 | NM_012476.2 |
| VPS13A | vacuolar protein sorting 13 homolog A | NM_033305.2 |
| ZNF577 | zinc finger protein 577 | NM_032679.2 |

All data were expressed relative to KLK2 as follows: samples with low KLK2 (counts <100) were removed (19/537), and data loge transformed.

Data was normalised to the housekeeping probes to the mean value of the probes GAPDH and RPLP2.

$${HK}_{i} = ( {x}_{i , GAPDH} + {x}_{i , RPLP 2} ) \text{/} 2$$

$${x}_{i , j} = \frac{\overline{HK}}{{HK}_{i}} \times {x}_{i , j}$$

Data were further normalised with the median of each probe across all samples adjusted to 1, with the interquartile range adjusted to that of KLK2:

$${x}_{i , j} = ( [ \frac{{x}_{i , j} + {Median}_{j}}{{IQR}_{j}} ] \times {IQR}_{KLK 2} \times {Median}_{KLK 2} ) \text{/} {x}_{i , KLK 2}$$

Where x_{i,j} is the expression value of sample i and probe j, Medianⱼ is the median expression value of probe j and IQRⱼ is the interquartile range of probe j. No correlation was seen with respect to patient's drugs, cohort site, urine pH, colour or sample volume (p>0.05; Chi-square and Spearman's Rank tests).

TABLE 3
Gene probes selected by LASSO in the original model
Gene transcript targets of NanoString ® probes in PUR model:

| | |
|---|---|
| AMACR | MEX3A |
| AMH | MMO1 |
| ANKRD34B | GDF15 |
| APOC1 | MMP11 |
| AR (exons 4-8) | MMP26 |
| MME | NKAIN1 |
| DPP4 | PALM3 |
| ERG (exons 4-5) | PCA3 |
| GABARAPL2 | PPFIA2 |
| GAPDH | SIM2 (short) |
| HOXC6 | SMIM1 |
| HPN | SSPO |
| IGFBP3 | SULT1A1 |
| IMPDH2 | TDRD |
| ITGBL1 | TMPRSS2/ERG fusion |
| KLK2 | TRPM4 |
| KLK4 | TWIST1 |
| MARCH5 | UPK2 |
| MED4 | |

TABLE 4
Gene probes selected by LASSO in an alternative model
Alternative gene transcript targets of NanoString ® probes in PUR model:

| | |
|---|---|
| AMACR | MEX3A |
| AMH | MIC1 |
| ANKRD34B | MMP26 |
| APOC1 | NKAIN1 |
| ARexons4-8 | PALM3 |
| CD10 | PCA3 |
| DPP4 | PPFIA2 |
| GABARAPL2 | SIM2.short |
| GAPDH | SMIM1 |
| HOXC6 | SSPO |
| HPN | SULT1A1 |
| IGFBP3 | TDRD |
| IMPDH2 | TMPRSS2/ERG fusion |
| ITGBL1 | TRPM4 |
| KLK4 | TWIST1 |
| MED4 | UPK2 |
| MEMO1 | |

TABLE 5
Gene probes selected by LASSO in a further alternative model
Alternative gene transcript targets of NanoString ® probes in PUR model:

| | |
|---|---|
| AMACR | MEMO1 |
| AMH | MEX3A |
| ANKRD34B | MIC1 |
| APOC1 | MMP11 |
| ARexons4.8 | MMP26 |
| CD10 | PALM3 |
| DPP4 | PCA3 |
| GAPDH | PPFIA2 |
| HOXC6 | SIM2.short |
| IGFBP3 | SLC12A1 |
| IMPDH2 | SSPO |
| KLK2 | SULT1A1 |
| KLK4 | TDRD |
| March5 | TMPRSS2.ERG.fusion |
| MED4 | UPK2 |

TABLE 6
Gene probes selected by LASSO in another alternative model
Alternative gene transcript targets of NanoString ® probes in PUR model:

| | |
|---|---|
| AMACR | MEM0O1 |
| AMH | MEX3A |
| ANKRD34B | MIC1 |
| APOC1 | PALM3 |
| ARexons4-8 | PCA3 |
| CD10 | SIM2.short |
| DPP4 | SMIM1 |
| ERG 3 ex 4-5 | TDRD |
| GABARAPL2 | TMPRSS2/ERG fusion |
| HOXC6 | TRPM4 |
| HPN | TWIST1 |
| IGFBP3 | UPK2 |
| ITGBL1 | |

### Example 4

### Model Production and Statistical Analysis

All statistical analyses and model constructions were undertaken in R version 3.4.123 [59] and unless otherwise stated, utilised base R and default parameters. The Prostate Urine Risk (PUR) signatures were constructed from the training set as follows: for each probe, a univariate cumulative link model was fitted using the R package clm with risk group as the outcome and NanoString® expression as inputs. Each probe that had a significant association with risk group (p<0.05) was used as input to the final multivariate model. A constrained continuation ratio model with an L1 penalisation was fitted to the training dataset using the glmnetcr library [60], an adaption of the LASSO method [61]. Default parameters were applied using the LASSO penalty and values from all probes selected by the univariate analysis used as input. The model with the minimum Akaike information criterion was selected. Where multiple samples were analysed from the same patient, the sample with the highest PUR-4 signature was used in survival analyses and Kaplan-Meier (KM) plots.

Ordinal logistic regression was undertaken using the ordinal R package [62]. Decision curve analysis (DCA) used the rmda R package [63]. Bootstrap adjustment of cohort to the prostate cancer prevalence figures reported in reference 64 for DCA was performed by: randomly sampling, with replacement, the Movember dataset according to the above proportions to construct a “new” dataset of 300 samples. This dataset construction was repeated 1000 times in total, with the net benefit of PUR-4 recorded for each dataset, again with the rmda package. The mean net benefit of PUR-4 and the treat-all options were used for plots. Survival analyses were performed using Cox proportional hazards models, the log-rank test and Kaplan-Meier estimators with time to progression by criteria described above as the end point.

Bootstrap resampling to assess significance of ROC analyses used the pROC package [65] for calculation, statistical tests and production of figures, with 1000 resamples used for tests. Random predictors were generated by randomly sampling from a uniform distribution between 0 and 1.

Decision curve analysis (DCA) [66] was performed to examine the net benefit of using PUR-signatures in the clinic. In order to undertake DCA that were representative of the general population, the prevalence of Gleason grades within our cohort were adjusted via bootstrap simulation to match that observed in a population of 219,439 men that were the control arm of the Cluster Randomised Trial of PSA Testing for Prostate Cancer (CAP) [64]. For the biopsied men within this CAP cohort, 23.6% were GG 1, 8.7% GG 2 or 3 and 7.1% GG 4 or greater, with a 60.6% of biopsies being prostate cancer negative. DCA was then undertaken on the resampled Movember dataset, and bootstrapping was repeated 1000 times, with net benefit recorded over each iteration.

The final DCA plots were then produced using the mean of results over all iterations to account for variance in sampling.

### Example 5

### Expression Results

The Clinical Cohort

The Movember cohort comprised 537 post-DRE urine samples from 504 patients collected from four centres (NNUH, n=312; RMH, n=121; Atlanta, n=87; Dublin, n=17). Men were categorised as having either No Evidence of Cancer (NEC, n=92) or localised prostate cancer at time of urine collection, as detected by TRUS biopsy (n=434), that were further subdivided into three risk categories using D'Amico criteria: Low (L), n=135; Intermediate (I), n=209; and High-risk (H), n=90.

Expression Assay Characteristics and Gene Panel

Prostate markers KLK2 and KLK3, were up to 28-fold higher in the EV fraction compared to sediment (paired samples Welch t-test p<0.001) and based on these analyses EVs were selected for further study.

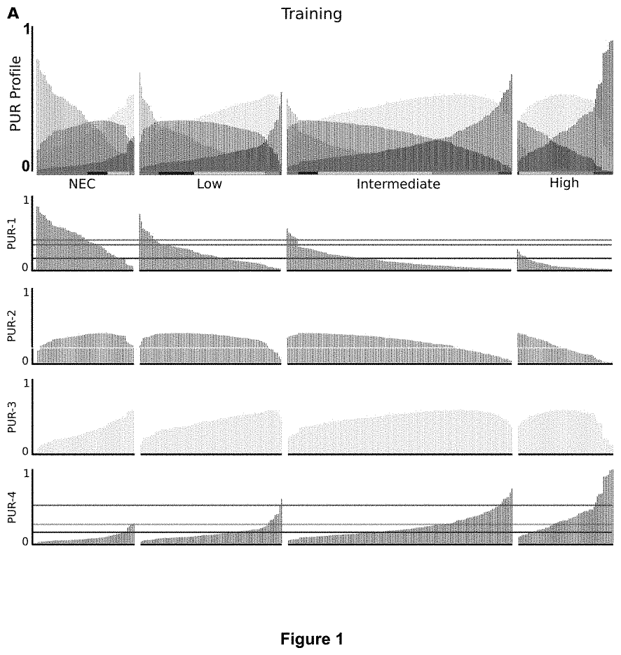

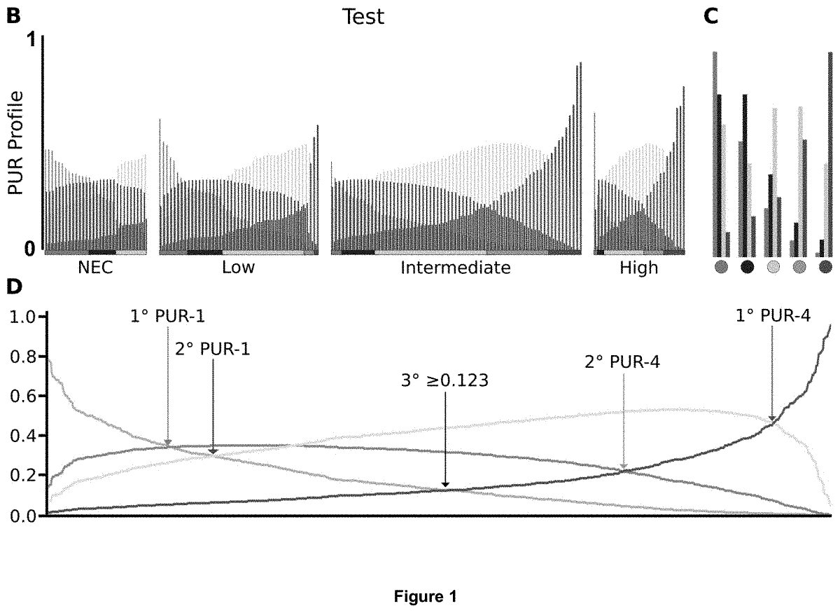

Median EV RNA yields for the NNUH cohort were similar for NEC (204 ng), Low- (180 ng) and Intermediate-risk (221 ng) patients, and lower in High-risk (108 ng) (Supplementary FIG. 1). Yields from three patients post-radical prostatectomy were 0.8-2 ng, suggesting that most EV RNA originates from the prostate.

### Example 6

### Development of the Prostate Urine Risk Signatures

Samples in D'Amico categories Low, Intermediate and High-risk, together with NEC samples were divided into the Movember Training set (two-thirds of samples; n=359) and the Movember Test set (one-third of samples; n=178) by random assignment stratified by risk category. Age, Stage, PSA, and GG were not significantly different across the two sets (p>0.05; Wilcoxon rank sum test/Fisher's Exact Test; Table 7).

TABLE 7
Patient characteristics

| Characteristics | Training | Test | p value |
|---|---|---|---|
| Patients, n | 359 | 178 | — |
| Collection centre: | | | |
| NNUH | 203 | 109 | — |
| RMH | 83 | 38 | — |
| Dublin | 9 | 8 | — |
| Atlanta | 64 | 23 | — |
| PSA, ng/ml, mean | 10.6 (6.9, 6.4) | 10.9 (6.9, 7) | 0.85 |
| (median; IQR) | | | |
| Age, yr, mean | 65.8 (67,11) | 67.2 (67,11) | 0.71 |
| (median; IQR) | | | |
| Family history of | 3.0, 6.1, 90.8 | 0.6, 6.2, 93.3 | 1 |
| prostate cancer, %; | | | |
| no, yes, NA | | | |
| First biopsy, n (%) | 298 (82.78) | 145 (81.46) | 1 |
| Prostate volume, ml; | 59.2 (49.8, 30.4) | 61.1 (49.2, 32.8) | 0.95 |
| mean (median; IQR) | | | |
| PSAD, ng/ml; ml, mean | 0.29 (0.19, 0.16) | 0.29 (0.18, 0.17) | 0.95 |
| (median; IQR) | | | |
| DRE, n | 107 | 52 | 1 |
| Diagnosis, n: | 358 | 177 | 0.9 |
| NEC, n (%) | 62 (17.3) | 30 (17.0) | — |
| D'Amico Low n (%) | 89 (24.9) | 45 (25.4) | — |
| D'Amico Intermediate n (%) | 139 (38.8) | 69 (39.0) | — |
| D'Amico High n (%) | 61 (17.0) | 27 (15.3) | — |
| Metastatic (bone scan) n (%)* | 7 (2.0) | 6 (3.3) | — |
| CAPRA, n; | 288 | 145 | 1 |
| Low (0-2) n (%) | 97 (33.7) | 49 (33.7) | — |
| Intermediate (3-5) n (%) | 108 (37.5) | 53 (36.6) | — |
| High (≥6) n (%) | 83 (28.8) | 43 (29.7) | — |
| Gleason, n: | 292 | 144 | 0.5 |
| Gs ≤ 6, n (%) | 119 (40.8) | 64 (44.4) | — |
| Gs = 7, n (%) | 131 (44.9) | 56 (38.9) | — |
| Gs >7, n (%) | 42 (14.4) | 24 (16.7) | — |

DRE = suspicious digital rectal examination;
Gs = Gleason score;
IQR = interguartile range;
NA = not available;
prostate cancer = prostate cancer;
PSA = prostate-specific antigen;
PSAD = prostate-specific antigen density;
TRUS = transrectal ultrasound.
NEC = No Evidence of Cancer/PSA normal for age or < 1 ng/ml.
*Metastatic men were diagnosed as High risk at time of urine collection. Percentages reported for Diagnosis, CAPRA and Gleason headings are calculated with the data available for that heading. For example, there are only 467 data available for CAPRA groupings out of the 588 patients.

The original model, as defined by the LASSO criteria in a constrained continuation ratio model, incorporated information from 37 probes (Table 3, for model coefficients see Table 8) and was applied to both training and test subject expression profiles (FIGS. 1A, B).

TABLE 8
Gene probes included as variables in the 37-gene PUR model
(Table 3) and their corresponding coefficients in the LASSO
regression

| PUR variable: | Coefficient |
|---|---|
| Intercept | −2.178157 |
| AMACR | 0.68299729 |
| AMH | 0.33631836 |
| ANKRD34B | 0.1673693 |
| APOC1 | 0.37122737 |
| AR (exons 4-8) | −0.4771042 |
| CD10 | −0.9433935 |
| DPP4 | −1.3364905 |
| ERG (exons 4-5) | 0.02561319 |
| GABARAPL2 | 0.51388528 |
| GAPDH | −0.9188083 |
| HOXC6 | 0.65430249 |
| HPN | −0.4625853 |
| IGFBP3 | −1.2101205 |
| IMPDH2 | 0.45431166 |
| ITGBL1 | −0.1094984 |
| KLK4 | −1.5051707 |
| March5 | −1.4391403 |
| MED4 | −1.0766399 |
| MEMO1 | −1.9473755 |
| MEX3A | 0.23180719 |
| MIC1 | 0.27927613 |
| MMP11 | 0.99181693 |
| MMP26 | 0.35495892 |
| NKAIN1 | 0.03529522 |
| PALM3 | 0.19549659 |
| PCA3 | 2.75492107 |
| PPFIA2 | −0.7369071 |
| SIM2.short | 0.90314335 |
| SMIM1 | −0.2209302 |
| SSPO | 0.92313638 |
| SULT1A1 | 1.7614731 |
| TDRD | 0.26666292 |
| TMPRSS2/ERG fusion | 0.47922694 |
| TRPM4 | 0.05947011 |
| TWIST1 | −0.2593533 |
| UPK2 | 0.63826112 |
| Cₚ 1 | 2.42583541 |
| Cₚ 2 | 1.48559352 |
| Cₚ 3 | −0.4792212 |

TABLE 9
Gene probes included as variables in the 33-gene PUR model
(Table 4) and their corresponding coefficients in the LASSO
regression

| PUR variable: | Coefficient |
|---|---|
| Intercept | −2.178157 |
| AMACR | 0.07162 |
| AMH | 0.353621 |
| ANKRD34B | 0.005572 |
| APOC1 | 0.137057 |
| ARexons4-8 | −0.06843 |
| CD10 | −0.03652 |
| DPP4 | −0.2321 |
| GABARAPL2 | −0.20102 |
| GAPDH | −0.30586 |
| HOXC6 | 0.131677 |
| HPN | 0.028676 |
| IGFBP3 | −0.04549 |
| IMPDH2 | 0.021572 |
| ITGBL1 | 0.017736 |
| KLK4 | −0.0853 |
| MED4 | −0.09181 |
| MEMO1 | −0.49072 |
| MEX3A | 0.030624 |
| MIC1 | 0.114047 |
| MMP26 | −0.08763 |
| NKAIN1 | 0.046038 |
| PALM3 | 0.137564 |
| PCA3 | 0.244057 |
| PPFIA2 | 0.024665 |
| SIM2.short | 0.17791 |
| SMIM1 | −0.11128 |
| SSPO | 0.384686 |
| SULT1A1 | 0.025707 |
| TDRD | 0.040212 |
| TMPRSS2/ERG fusion | 0.10908 |
| TRPM4 | 0.075311 |
| TWIST1 | −0.39993 |
| UPK2 | 0.076676 |
| Cₚ 1 | 10.54831565 |
| Cₚ 2 | 9.32739569 |
| Cₚ 3 | 7.04942643 |

TABLE 10
Gene probes included as variables in the 29-gene PUR model
(Table 5) and their corresponding coefficients in the LASSO
regression

| PUR variable: | Coefficient |
|---|---|
| Intercept | −2.178157 |
| AMACR | 0.383005 |
| AMH | 0.124671 |
| ANKRD34B | 0.093695 |
| APOC1 | 0.28606 |
| ARexons4.8 | −0.39105 |
| CD10 | −0.63788 |
| DPP4 | −0.97386 |
| GAPDH | −0.28459 |
| HOXC6 | 0.485867 |
| IGFBP3 | −0.90499 |
| IMPDH2 | 0.35457 |
| KLK4 | −1.195 |
| March5 | −0.9502 |
| MED4 | −0.83134 |
| MEMO1 | −1.49625 |
| MEX3A | 0.083018 |
| MIC1 | 0.105871 |
| MMP11 | 0.674445 |
| MMP26 | 0.234515 |
| PALM3 | 0.139616 |
| PCA3 | 2.501731 |
| PPFIA2 | −0.44841 |
| SIM2.short | 0.833267 |
| SLC12A1 | 0.005144 |
| SSPO | 0.615141 |
| SULT1A1 | 1.379276 |
| TDRD | 0.183405 |
| TMPRSS2.ERG.fusion | 0.474497 |
| UPK2 | 0.383788 |
| Cₚ 1 | 2.255048 |
| Cₚ 2 | 1.407897 |
| Cₚ 3 | −0.4463 |

TABLE 11
Gene probes included as variables in the 25-gene PUR model
(Table 6) and their corresponding coefficients in the LASSO
regression

| PUR variable: | Coefficient |
|---|---|
| Intercept | −2.178157 |
| AMACR | 0.079281 |
| AMH | 0.055753 |
| ANKRD34B | 0.07382 |
| APOC1 | 0.180496 |
| ARexons4-8 | −0.17182 |
| CD10 | −0.01629 |
| DPP4 | −0.3026 |
| ERG 3 ex 4-5 | 0.038413 |
| GABARAPL2 | −0.31826 |
| HOXC6 | 0.065652 |
| HPN | 0.050407 |
| IGFBP3 | −0.10451 |
| ITGBL1 | 0.029658 |
| MEMO1 | −0.30408 |
| MEX3A | 0.065026 |
| MIC1 | 0.028617 |
| PALM3 | 0.070976 |
| PCA3 | 0.247588 |
| SIM2.short | 0.067356 |
| SMIM1 | −0.02115 |
| TDRD | 0.072277 |
| TMPRSS2/ERG fusion | 0.028723 |
| TRPM4 | 0.031403 |
| TWIST1 | −0.08686 |
| UPK2 | 0.044997 |
| Cₚ 1 | 8.323515976 |
| Cₚ 2 | 7.35799112 |
| Cₚ 3 | 5.109392713 |

For each sample the 4-signature PUR-model defined the probability of containing NEC (PUR-1), L (PUR-2), I (PUR-3) and H (PUR-4) material within samples (FIGS. 1A, B). The sum of all four PUR-signatures in any individual sample was 1 (PURI+PUR2+PUR3+PUR4=1). The strongest PUR-signature for a sample was termed the primary (1°) signature while the second highest was called the secondary signature (2°; FIGS. 1C, D).

Pre-Biopsy Prediction of D'Amico Risk, CAPRA Score and Gleason

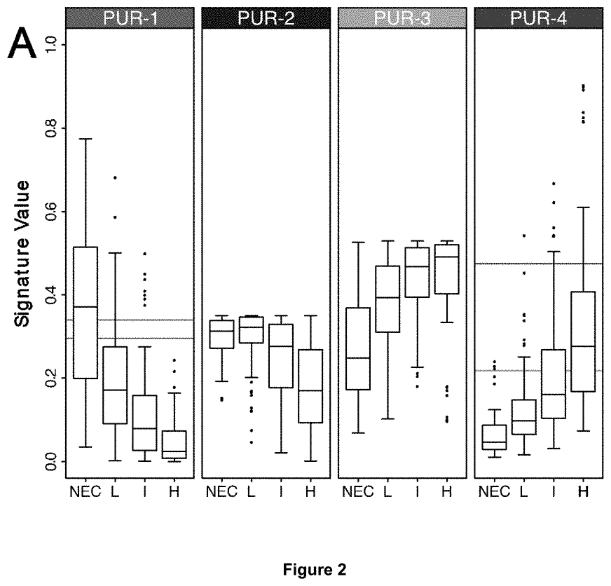

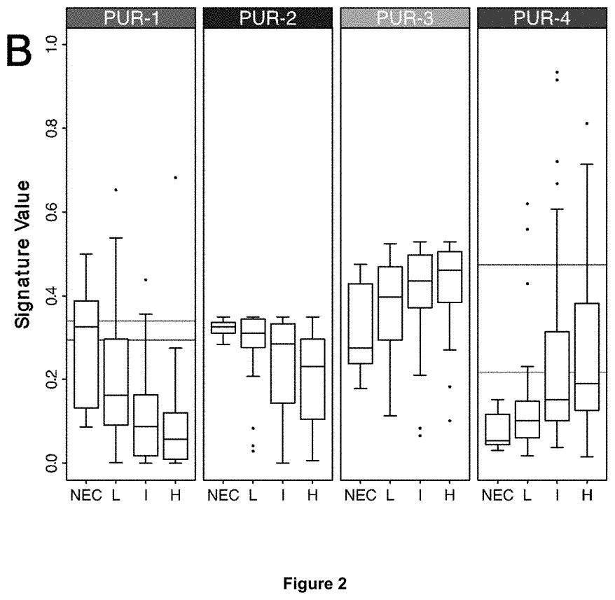

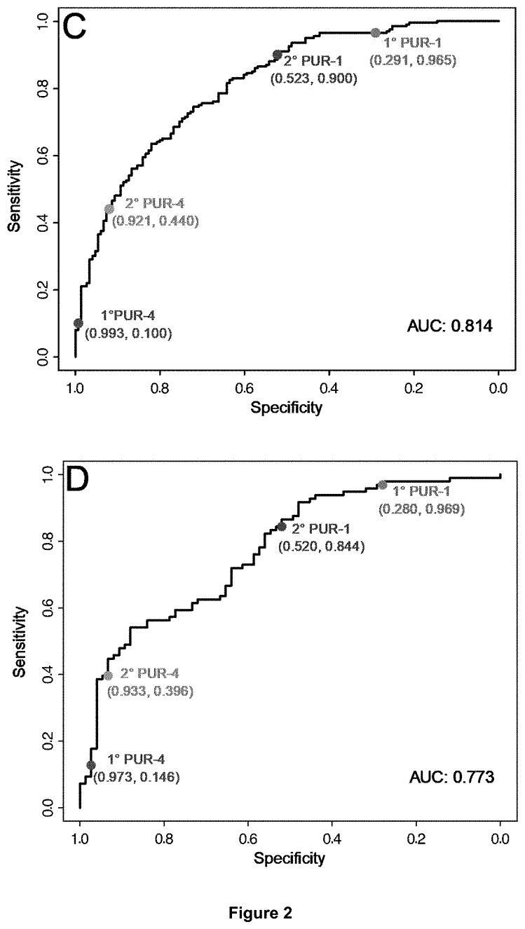

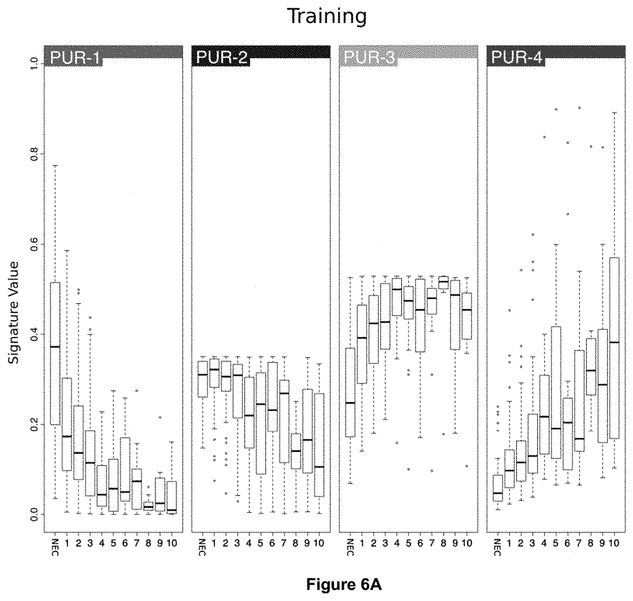

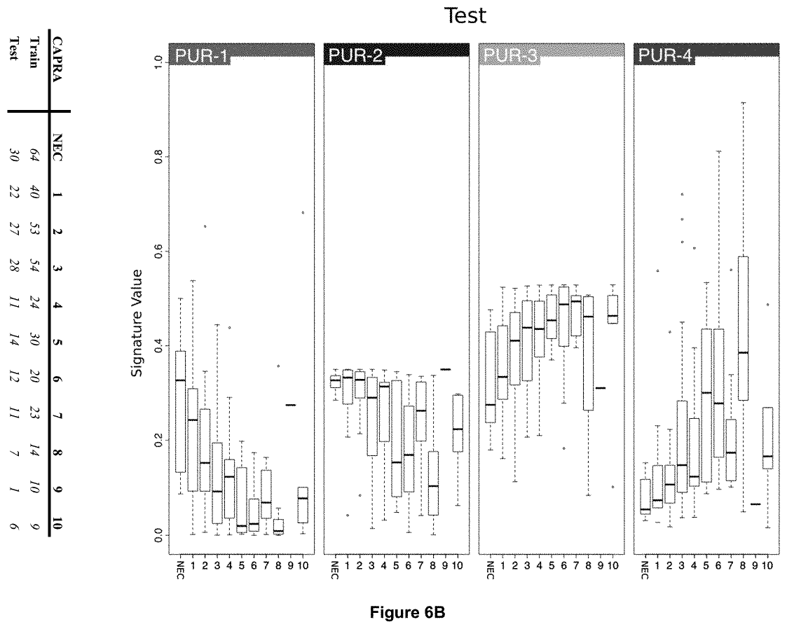

Primary PUR-signatures (PUR-1 to 4) were found to significantly associate with clinical category (NEC, L, I, H respectively) in both training and test sets (p<<0.001, Wald test, ordinal logistic regression in both Training and Test subject datasets, FIGS. 2A, B). A similar association was observed with CAPRA score (p<<0.001, Wald test, ordinal logistic regression in both Training and Test subject datasets; FIG. 6).

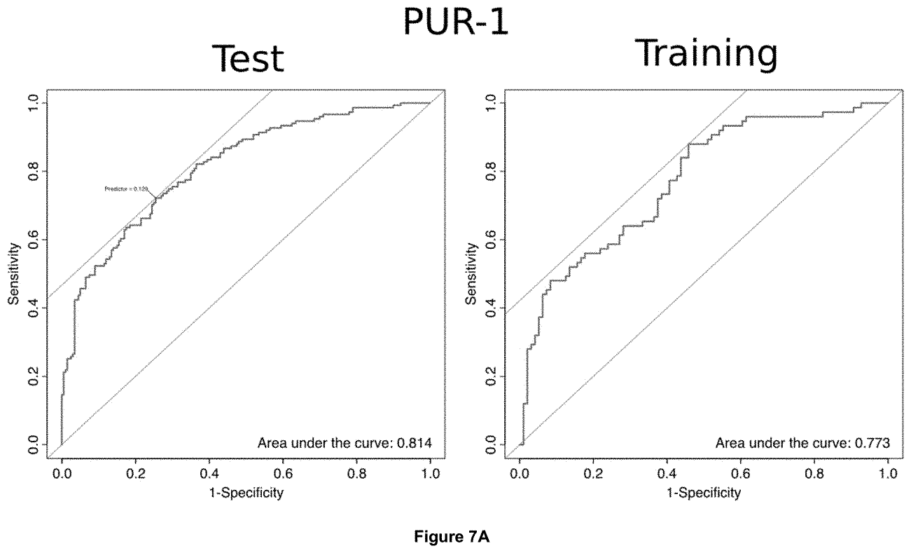

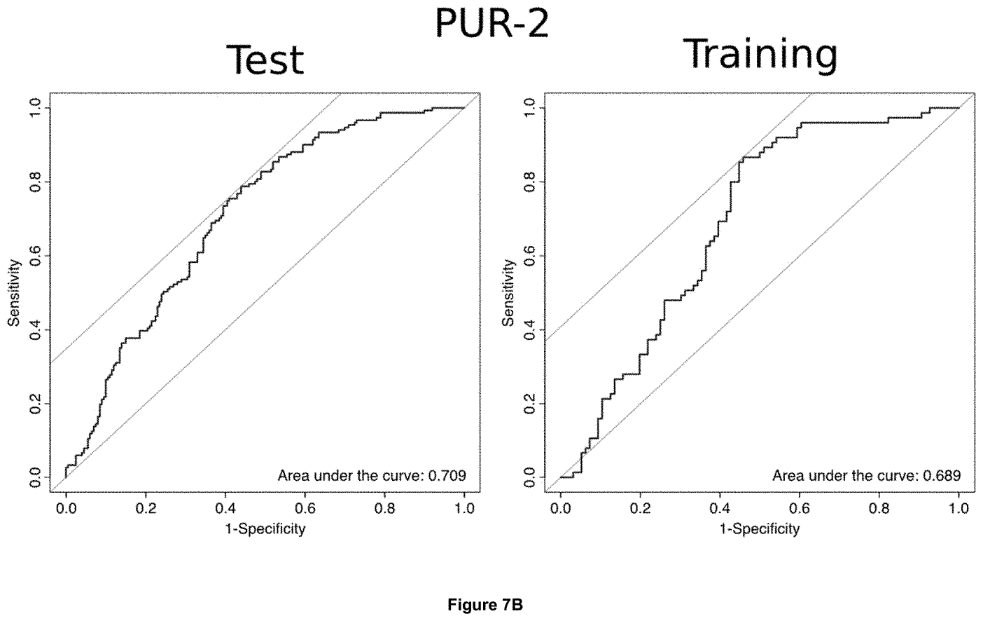

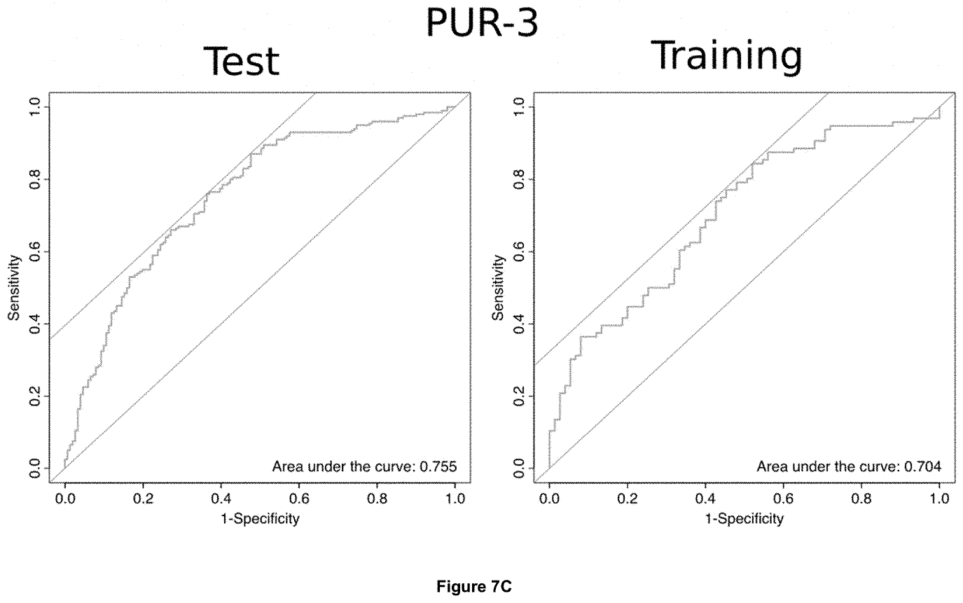

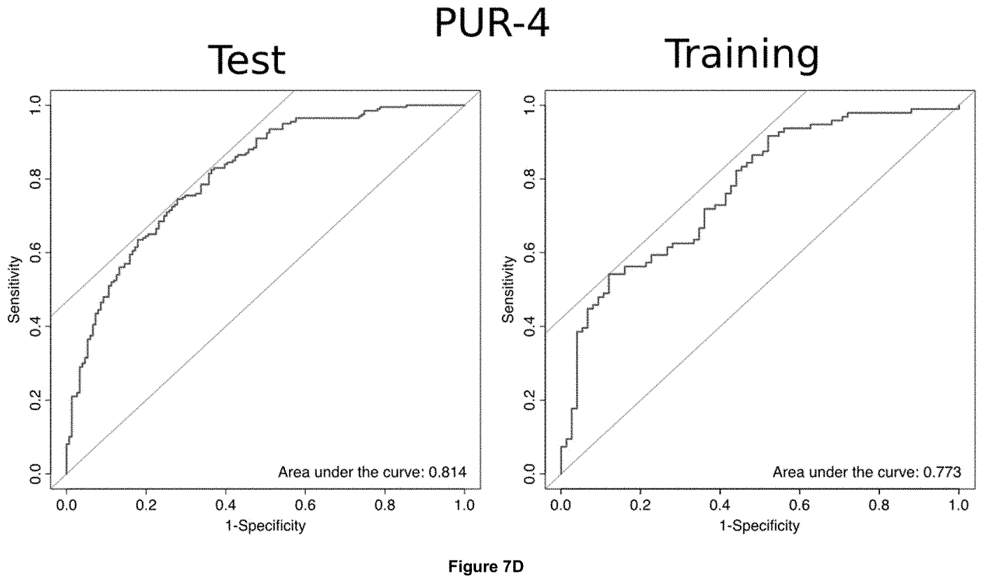

Based on recommended guidelines [4,5,6], the distinction between D'Amico low and intermediate-risk is considered critical because radical therapy is commonly recommended for patients with high and intermediate-risk cancer. We therefore initially tested the ability of the PUR-model to predict the presence of H or I disease (H+I) compared to L+NEC. Each of the four PUR-signatures alone were able to predict the presence of significant disease (Risk category≥Intermediate, Area Under the Curve (AUC)≥0.68 for each PUR signature, test; FIG. 7), and were significantly better than a random predictor (p<0.001, DeLong's test). However, PUR-1 and PUR-4 were best and equally effective at discerning significant disease; AUCs for both PUR-4 and for PUR-1 in the Training and Test cohorts were respectively 0.818 and 0.783 (FIGS. 2C & D).

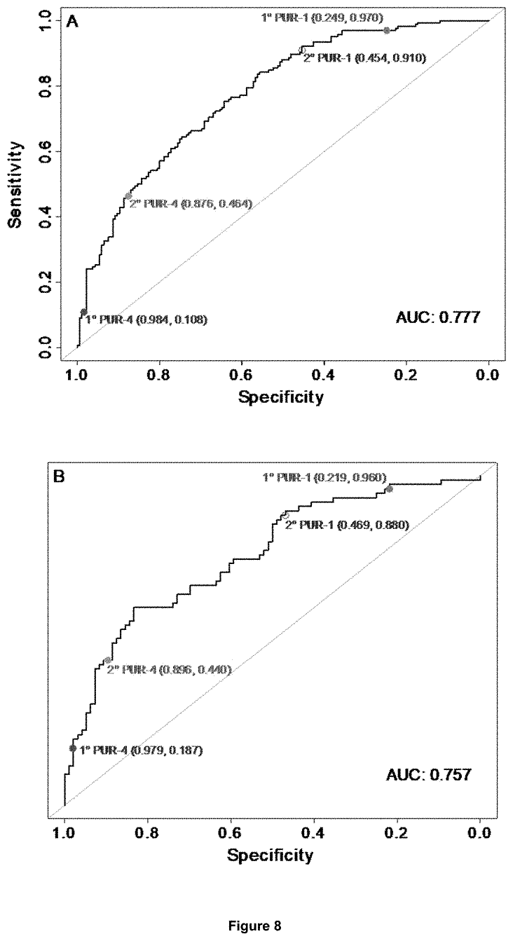

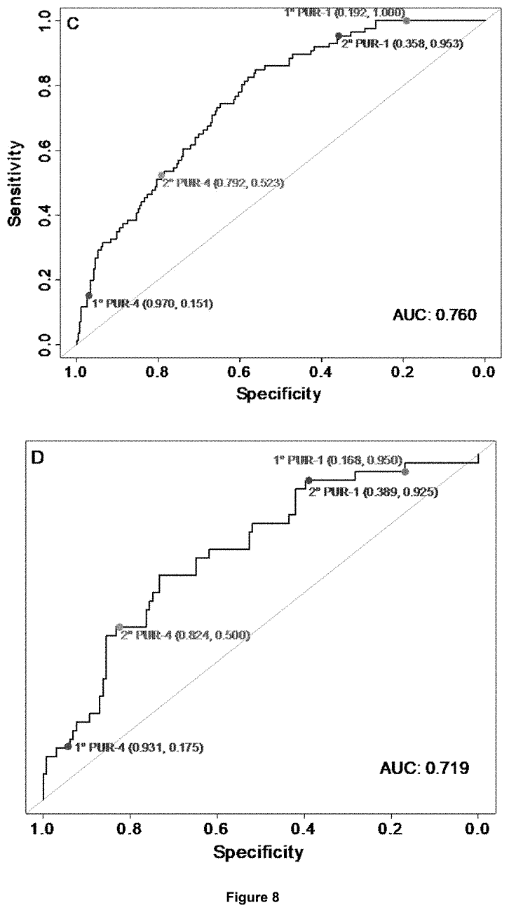

When Gleason Grade alone was considered we found that PUR-4 predicted GG ≥2 with AUCs of 0.77 (Train) and 0.76 (Test) and Gs≥4+3 with AUCs of 0.76 (Train) and 0.76 (Test) (FIG. 8). The ability to predict Gs≥7 was particularly relevant because this was chosen as an endpoint for aggressive disease in previous urine biomarker studies, where AUCs of 0.78, 0.77 and 0.74 were reported in references 18, 19 and 21 respectively.

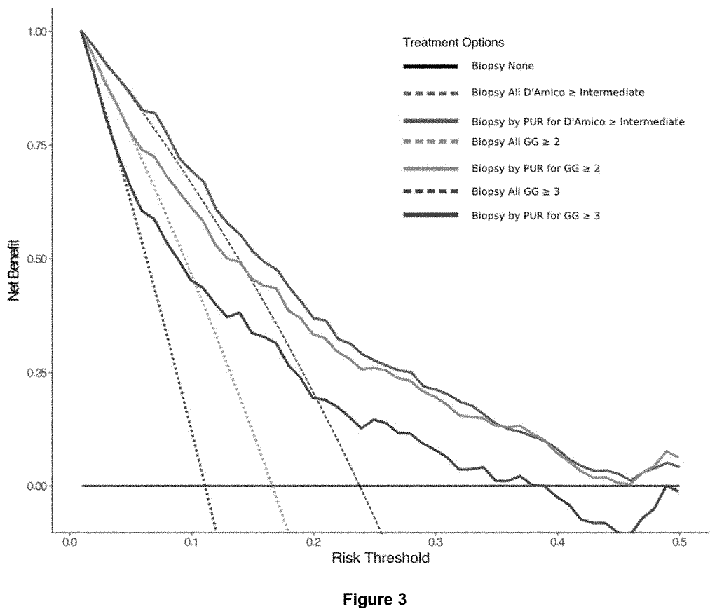

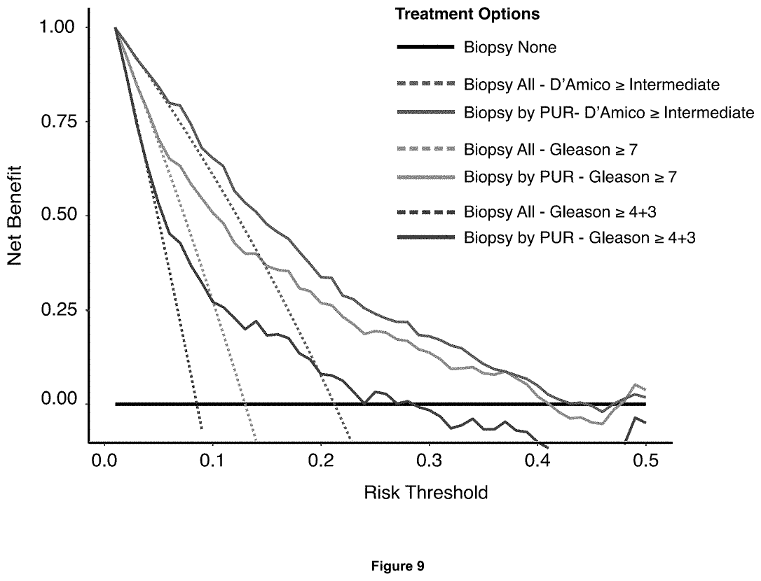

Decision curve analysis (DCA) [27] was performed to examine the net benefit of using PUR-signatures in a non-PSA screened population. Biopsy of men based upon their PUR-4 score provided a net benefit over biopsy of men based on current clinical practice across all thresholds (FIG. 3). When DCA was also undertaken within the context of a PSA-screened population, PUR continued to provide a net benefit (FIG. 9).

Active Surveillance Cohort

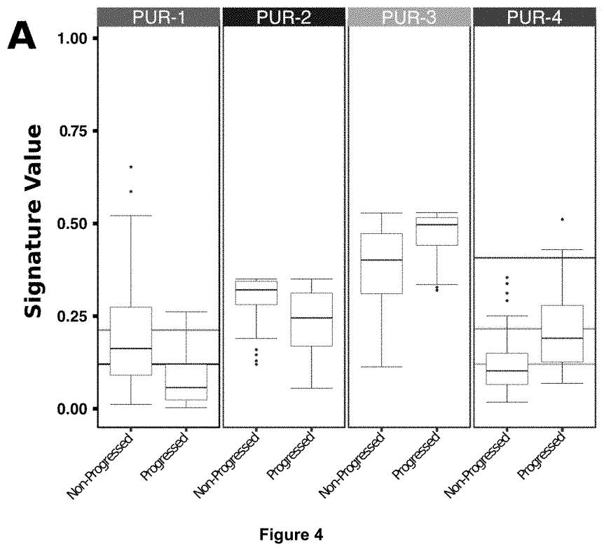

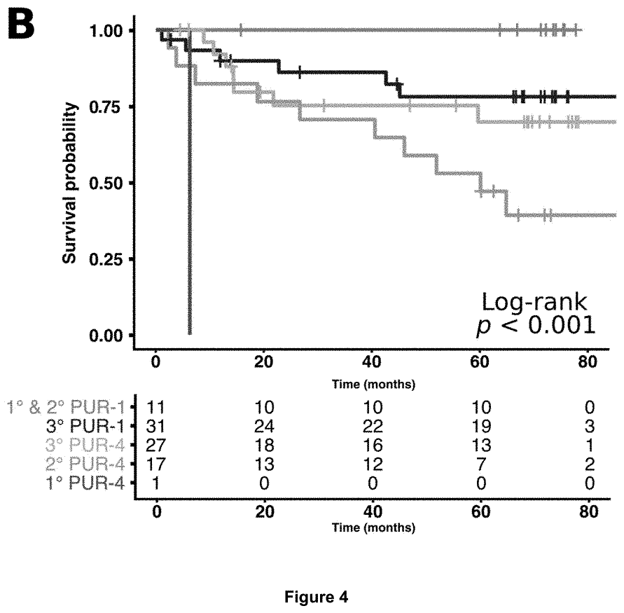

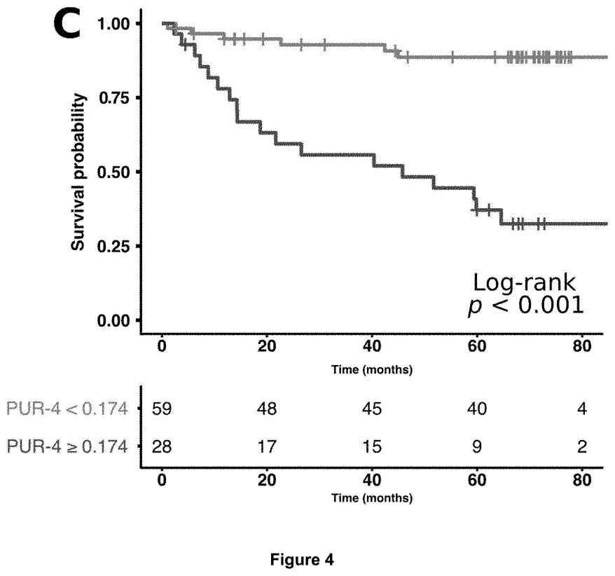

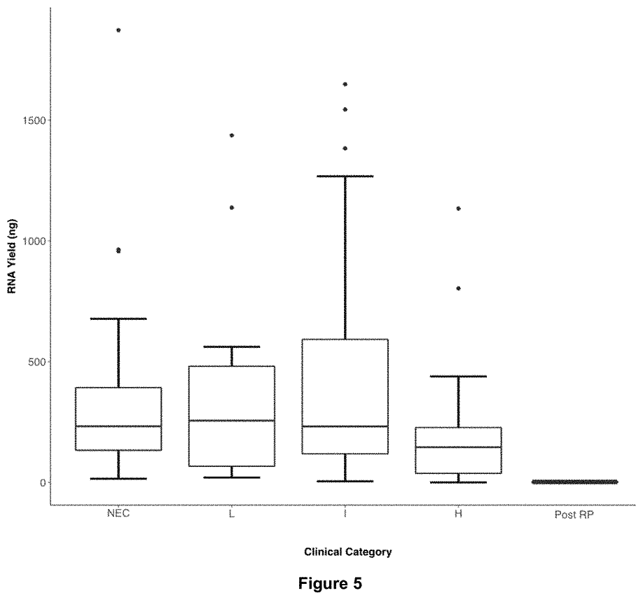

Within the Movember cohort were 120 samples from 87 men enrolled in AS at the Royal Marsden Hospital, UK. The median follow-up from urine sample collection was 5.7 years (range 5.1-7.0 years). The median time from sample collection to clinical progression or final follow up was 503 days (range 0.1-7.4 years). The PUR profiles were significantly different between the 23 men who progressed within five years of urine sample collection, and 49 men who did not progress (p<<0.001, Wilcoxon rank sum test; FIG. 4A). Twenty two men progressed by MP-MRI criteria, with 9 men progressing based on MP-MRI alone.

Calculation of the Kaplan-Meier plots with samples divided on the basis of 1°, 2° and 3° PUR-1 and PUR-4 signatures showed significant differences in clinical outcome (p<<0.001, log-rank test, FIG. 4B, log-rank test p<0.05 in 93.585% of 100,000 cohort resamples with replacement. Proportion of PUR-4, a continuous variable, had a significant association with clinical outcome (p<<0.001; IQR HR=5.867 (95% CI: 1.683-20.455)); Cox Proportional hazards model). A robust optimal threshold of PUR-4 was determined to dichotomise AS patients into two groups (PUR-4=0.174, based on the median optimal threshold to minimise Log rank test p-value from 1000 resampling of the cohort with replacement). The two groups had a large difference in time to progression (p<<0.001, log-rank test, FIG. 4C, HR=8.230 (95% CI: 3.255-20.810)); 60% progression within 5 years of urine sample collection in the poor prognosis group compared to 10% in the good prognosis group. This result is robust (p<0.05 in 99.838% of 100,000 cohort resamples with replacement.

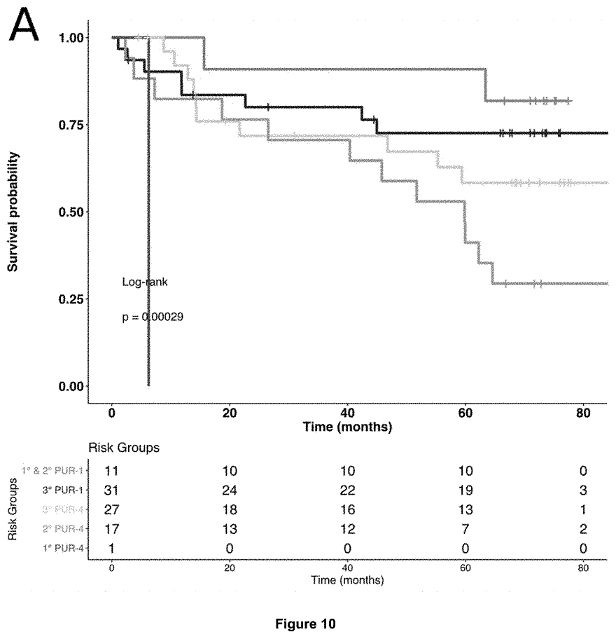

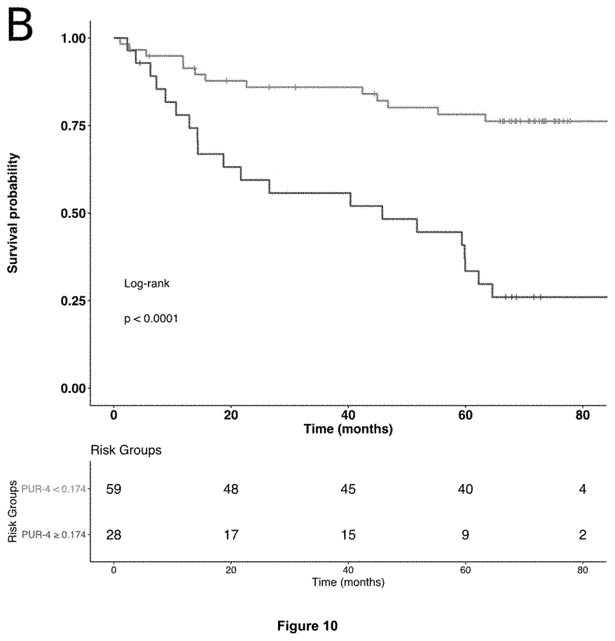

When progression via MP-MRI criteria was also included, both primary PUR-status and dichotomised PUR threshold remained a significant predictor of progression (p<<0.001 log-rank test, FIG. 10).

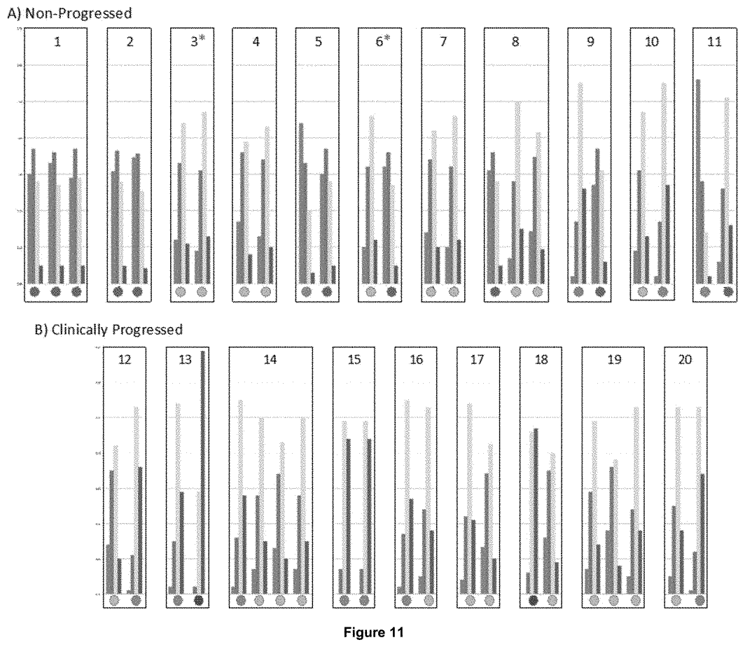

For 20 of the men entered into the AS trial multiple urine specimens had been collected, allowing us to assess the stability of urine profiles over time (FIG. 11). In patients that had not progressed, samples were found to be stable compared to a null model generated by randomly selected samples from the whole Movember Cohort (p=0.011; bootstrap analysis with 100,000 iterations). Samples from men deemed to have progressed failed this stability test (p=0.059), indicating greater variability between samples in this patient group.

### Example 7

### Radical Prostatectomy Data

The histological patterns of prostate tumours are assessed by a pathologist and given a Gleason grading for severity of disease, ie Gleason 3, 4 and 5 tumour. This is then used to calculate a Gleason score for the patient.

The rules for calculating the Gleason scores are different for biopsies and radical prostatectomies.
Gleason score is potentially 2 to 10, the sum of the two most prevalent Gleason patterns: primary and secondary patterns
If only one pattern is present, the primary and secondary patterns are given the same grade
Needle biopsy sets contain cores from different anatomically designated sites
It is recommended that the Gleason score be assigned separately for each anatomically designated site, since information is lost if only a global score is given
Any glands showing perineural invasion should be excluded in assigning Gleason grading because perineural invasion distorts gland morphology such that Gleason 3 glands can resemble Gleason 4

Assignment of Patterns:
Recommendations are based on 2005 International Society of Urological Pathology (ISUP) Consensus Conference on Gleason Grading [67]
Some specimens may show a pattern that is the third most prevalent, and this is called a tertiary pattern
Needle biopsy: the most prevalent pattern (commonest) is graded as primary, and the worst pattern (even if it is third most prevalent) is graded as secondary
Radical prostatectomy: Gleason score should be based on the primary and secondary patterns (commonest and next commonest) with a tertiary given also if required which does not contribute to the score.

So a prostate can have a Gleason score of, for example, 3+3=6, or 3+4=7, or 4+3=7, or 4+5=9, or other combinations.

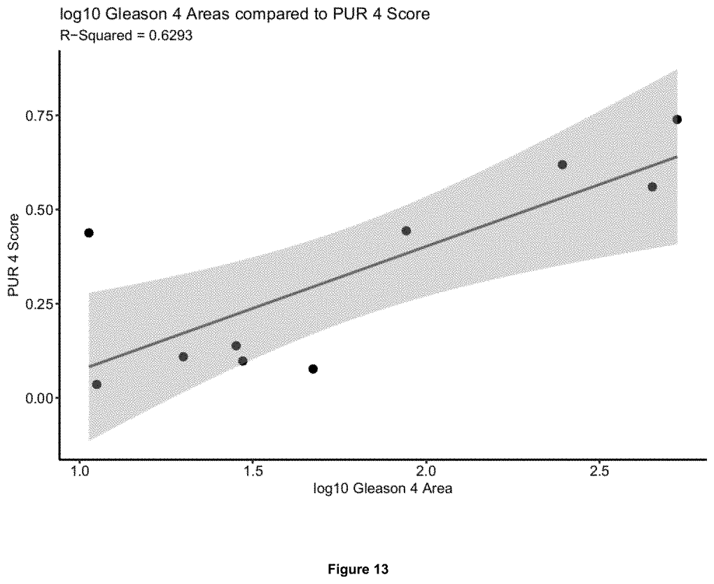

Total area of Gleason 4 in prostates from the radical prostatectomies were assessed as follows:
Each prostate was cut into ˜1 cm thick slices.
Thin sections were then taken from one side of each 1 cm thick slice, mounted on a slide and H&E stained.
The slides were then examined by a pathologist, who drew around all the areas of tumour. The pathologist then examined all the tumour areas in detail for Gleason 3, 4 and 5 content. It is common for Gleason 4 and Gleason 3 tumour to be intermingled, therefore a score was provided for the % of Gleason 3 and Gleason 4 in each tumour area.
The stained sections were then scanned and software (such as imageJ or Fiji) was used to calculate the tumour areas in mm².
The calculated tumour area was multiplied by, for example, the percentage of Gleason 4 in that area to get an approximate area of Gleason 4 for each tumour focus (Table 12). The results of the individual tumour foci can then be added up to get a figure for the total area of Gleason 3, Gleason 4 and Gleason 5 in each prostate, and these can be plotted against the PUR signatures (e.g. FIG. 13).

It can be seen that the PUR-4 signal correlates to the total area of Gleason 4 (FIG. 13) but not to total tumour area or Gleason 3 area. Only one of the prostates had some Gleason 5, so it was not possible to plot that comparison.

The PUR signal is noticeably higher than the G4 area in sample 44_3. One explanation for this may be the presence of a small area of G5 in this prostate.

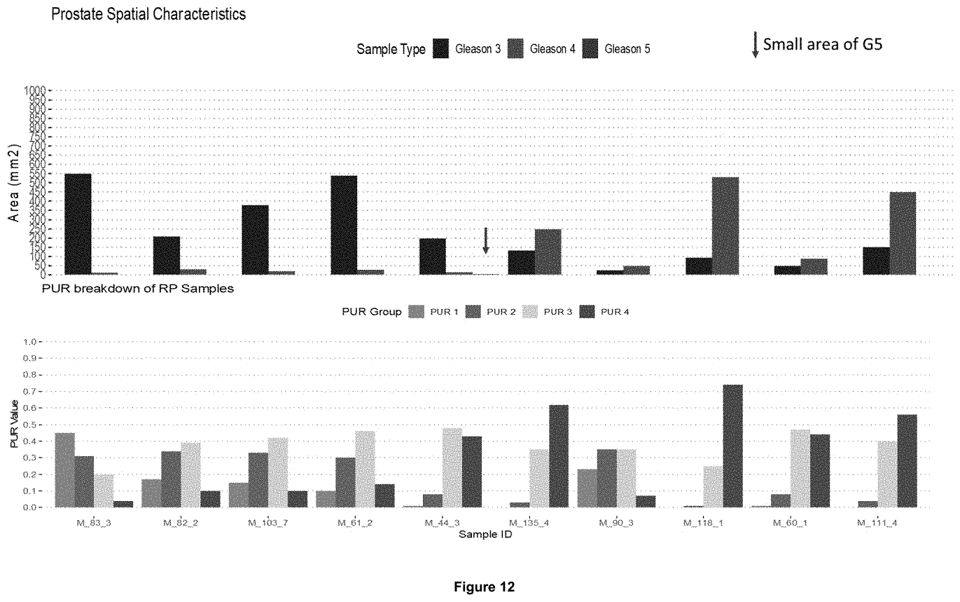

TABLE 12
Data for the radical prostatectomy samples shown in FIG. 12 with respect to PUR-4 signature,
biopsy Gleason scores and radical prostatectomy Gleason scores. These are the data used to generate the
correlation shown in FIG. 13. As can be seen, four of the biopsy Gleason scores are lower than what was
found in the radical prostatectomy, and one was higher in the biopsy than the radical prostatectomy.

| | | | | | | Total | Total | | | | | |
|---|---|---|---|---|---|---|---|---|---|---|---|---|
| | | | | | Rad | Prostate | Tumour | | | Area of | Area of | |
| | | D'Amico | D'Amico on | Biopsy | Prost | Area | Area | | | G4 | G3 | |
| Sample | PSA | on Biopsy | Rad Prost | Gs | Gs | (mm2) | (mm2) | % G4 | % G3 | (mm2) | (mm2) | PUR-4 |
| M_83_3 | 5.5 | Low | Low | 3 + 3 | 3 + 3 | 5180 | 560 | 2 | 98 | 11 | 549 | 0.04 |
| M_82_2 | 5.2 | Int | Int | 3 + 4 | 3 + 4 | 3861 | 237 | 13 | 88 | 30 | 207 | 0.10 |
| M_103_7 | 15.0 | Int | Int | 3 + 3 | [symbol] | 3373 | 399 | 5 | 95 | 20 | 379 | 0.11 |
| M_61_2 | 5.8 | Low | [symbol] | 3 + 3 | [symbol] | 4699 | 566 | 5 | 95 | 28 | 538 | 0.14 |
| M_44_3 | 8.4 | Int | Int | [symbol] | 3 + 4 | 4817 | 213 | 5 | 95 | 11 | 202 | 0.44 |
| M_135_4 | 6.7 | Low | [symbol] | 3 + 3 | [symbol] | 5895 | 380 | 65 | 35 | 247 | 133 | 0.62 |
| M_90_3 | 10.3 | Int | Int | 3 + 4 | [symbol] | 13404 | 73 | 65 | 35 | 47 | 25 | 0.08 |
| M_118_1_Pre | 8.2 | Int | Int | 4 + 3 | 4 + 3 | 4651 | 623 | 85 | 15 | 530 | 93 | 0.75 |
| M_60_1 | 7.4 | Int | Int | 4 + 3 | 4 + 3 | 3679 | 135 | 65 | 35 | 88 | 47 | 0.44 |
| M_111_4 | 19.1 | Int | Int | 4 + 3 | 4 + 3 | 4464 | 599 | 75 | 25 | 449 | 150 | 0.56 |

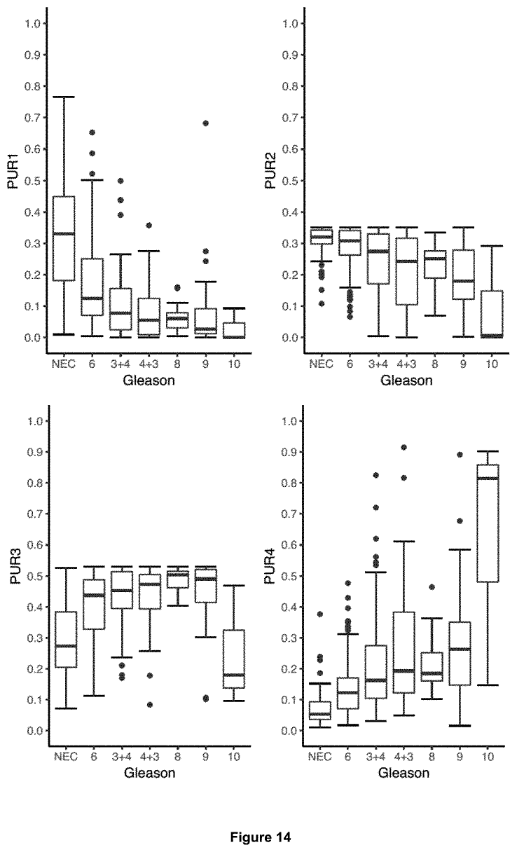

These data fit with PUR4 being able to predict disease progression, for example in men under active surveillance, which to a large extent is down to increasing amounts of Gleason 4 [68,69]. These data also fit the association of increasing PUR-4 signal with increasing Gleason score in TRUS biopsy (FIG. 14)

References 68 and 69 show that time to biochemical recurrence/PSA failure after treatment of Gleason score 7 tumours is related to the total amount of Gleason 4 tumour. Therefore, a test that can predict the amount of Gleason 4 without having to undergo a radical prostatectomy would be clinically valuable.

MRI is commonly used to predict this, but it has a high rate of false positives, and also does not pick up some disease. Therefore, using the PUR signature as a predictor of Gleason 4 amount, or significant Intermediate or High risk disease, either alone, or in combination with MRI could improve accuracy and reduce the number of unnecessary biopsies taken. These radical prostatectomy data demonstrate that the PUR-4 signature is potentially a better predictor of Gleason 4 content than biopsy.

Around 20-30% of TRUS biopsy Gleason scores change following radical prostatectomy, (mostly to more severe) and Gleason score does not necessarily correlate to the actual amount of tumour, therefore the correlation between PUR-4 and disease status was predicted to be clearer in the radical prostatectomy data, rather than the biopsy data, which it appears to be.

TABLE 13
Example Control Genes: Prostate specific control transcripts

| | | | | |
|---|---|---|---|---|
| KLK2 | PCA3 | ACPP | PMA | SPINK1 |
| KLK3 | TMPRSS2 | PTI-1 | HOXB13 | |
| KLK4 | TMPRSS2/ERG | PSCA | PMEPA1 | |
| FOLH1(PSMA) | TGM4 | NKX3.1 | PAP | |
| PCGEM1 | RLN1 | SPDEF | STEAP1 | |

TABLE 14
Example Control Genes: House Keeping Control genes

| | | | | | | | |
|---|---|---|---|---|---|---|---|
| HPRT | PSMB4 | TFR | RPS16 | IMPDH1 | ATP5F1 | RPL7a | CLTC |
| B2M | RAB7A | RPS13 | RPL4 | IDH2 | H2A.X | RNAP II | |
| TBP | REEP5 | RPL27 | RPL6 | KGDHC | IMP | RPL10 | |
| GAPDH | 18S rRNA | RPS20 | OAZ1 | SRF7 | accession | RPL23a | |
| ALAS1 | 28s rRNA | RPL30 | RPS12 | RPLP0 | ODC-AZ | RPL37 | |
| RPLP2 | PBGD | RPL13A | LDHA | ALDOA | PDHA1 | RPS11 | |
| KLK3_ex2-3 | ACTB | RPL9 | PGAM1 | COX IV | PLA2 | RPS3 | |
| KLK3_ex1-2 | UBC | SRP14 | PGK1 | AST | PMI1 | SDHB | |
| SDH1 | rb 23kDa | RPL24 | VIM | MDH | SRP75 | SNRPB | |
| GPI | TUBA1 | RPL22 | PFKP | EIF4A1 | RPL3 | SDH | |
| PSMB2 | RPS9 | RPS29 | EF-1d | FH | RPL32 | TCP20 | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, the described embodiments are to be considered in all respects only as illustrative and not restrictive. All similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that use of such terms and expressions imply excluding any equivalents of the features shown and described in whole or in part thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Clauses

The present invention additionally provides the following clauses, listed as numbered embodiments, which may be combined with other features and aspects of the invention:
1. A method of providing a cancer diagnosis or prognosis based on the expression status of a plurality of genes comprising:
   (a) providing a plurality of patient expression profiles each comprising the expression status of the plurality of genes in at least one sample obtained from each patient, wherein each of the patient expression profiles is associated with one or more cancer risk groups, wherein each cancer risk group is associated with a different cancer prognosis or cancer diagnosis, optionally wherein each patient expression profile is normalised relative to (i) the expression status of one or more normalising genes in the same patient sample, (ii) an average expression status of one or more normalising genes in a reference population and/or (iii) the status of one or more control-probes;
   (b) counting the number (n) of different cancer risk groups to which the patient expression profiles belong, optionally wherein at least one cancer risk group is associated with an absence of cancer;
   (c) applying a cumulative link model to the patient expression profiles to select a subset of one or more genes from the plurality of genes in the patient expression profile that are significantly associated with the n cancer risk groups; and
   (d) inputting the expression values of the selected subset of one or more genes to a constrained continuation ratio logistic regression model comprising n modifier coefficients such that the model generates n risk scores for each patient expression profile, wherein for each patient expression profile, a risk score is provided for each of the n cancer risk groups and wherein each of the n risk scores for a given patient expression profile is associated with the likelihood of membership to the corresponding cancer risk group, optionally wherein the regression model generates regression coefficients associated with each of the selected subset of genes based on the plurality of patient expression profiles.
2. A method of classifying prostate cancer in a test subject or identifying a test subject with a poor prognosis for cancer based on the expression status of a plurality of genes comprising:
   (a) providing a plurality of patient expression profiles each comprising the expression status of the plurality of genes in at least one sample obtained from each patient, wherein each of the patient expression profiles is associated with one or more cancer risk groups, wherein each cancer risk group is associated with a different cancer prognosis or cancer diagnosis, optionally wherein each patient expression profile is normalised relative to (i) the expression status of one or more normalising genes in the same patient sample, (ii) an average expression status of one or more normalising genes in a reference population and/or (iii) the status of one or more control-probes;
   (b) counting the number (n) of different cancer risk groups to which the patient expression profiles belong, optionally wherein at least one cancer risk group is associated with an absence of cancer;
   (c) applying a cumulative link model to the patient expression profiles to select a subset of one or more genes from the plurality of genes in the patient expression profile that are significantly associated with the n cancer risk groups;
   (d) inputting the expression values of the selected subset of one or more genes to a constrained continuation ratio logistic regression model comprising n modifier coefficients such that the model generates n risk scores for each patient expression profile, wherein for each patient expression profile, a risk score is provided for each of the n cancer risk groups and wherein each of the n risk scores for a given patient expression profile is associated with the clinical outcome of the corresponding cancer risk group and wherein the regression model generates regression coefficients associated with each of the selected genes based on the plurality of patient expression profiles;
   (e) providing a test subject expression profile comprising the expression status of the same selected subset of one or more genes as in step (c) in at least one sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
   (f) inputting the test subject expression profile to the constrained continuation ratio logistic regression model comprising the n modifier coefficients and gene regression coefficients generated in step (d) to generate n risk scores for the test subject expression profile, wherein each of the n risk scores for the test subject expression profile is associated with the likelihood of membership to the corresponding cancer risk group; and
   (g) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.
3. A method of classifying prostate cancer in a test subject or identifying a test subject with a poor prognosis for cancer comprising:
   (a) providing a test subject expression profile comprising the expression status of a subset of one or more genes selected by a method according to the first aspect of the invention in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
   (b) inputting the test subject expression profile to a constrained continuation ratio logistic regression model comprising the n modifier coefficients and gene regression coefficients generated using a method according to the first aspect of the invention, thereby generating n risk scores, wherein each of the n risk scores for a given test subject expression profile is associated with the likelihood of membership to the corresponding cancer risk group, wherein the n modifier coefficients and corresponding gene regression coefficients are generated by applying the regression model to patient expression profiles comprising the expression status of the same subset of one or more genes; and
   (c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.
4. A method of classifying prostate cancer in a test subject or identifying a test subject with a poor prognosis for cancer comprising:
   (a) providing a test subject expression profile comprising the expression status of a plurality of the 37 genes in Table 3 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
   (b) inputting the test subject expression profile to a constrained continuation ratio logistic regression model comprising the 4 modifier coefficients (Cp1, Cp2, Cp3 and the intercept) and 36 gene regression coefficients in Table 8, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
   (c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.
5. A method of classifying prostate cancer in a test subject or identifying a test subject with a poor prognosis for cancer comprising:
   (a) providing a test subject expression profile comprising the expression status of a plurality of the 33 genes in Table 4 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
   (b) inputting the test subject expression profile to a constrained continuation ratio logistic regression model comprising the 4 modifier coefficients (Cp1, Cp2, Cp3 and the intercept) and 33 gene regression coefficients in Table 9, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
   (c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.
6. A method of classifying prostate cancer in a test subject or identifying a test subject with a poor prognosis for cancer comprising:
   (a) providing a test subject expression profile comprising the expression status of a plurality of the 29 genes in Table 5 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
   (b) inputting the test subject expression profile to a constrained continuation ratio logistic regression model comprising the 4 modifier coefficients (Cp1, Cp2, Cp3 and the intercept) and 29 gene regression coefficients in Table 10, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
   (c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.
7. A method of classifying prostate cancer in a test subject or identifying a test subject with a poor prognosis for cancer comprising:
   (a) providing a test subject expression profile comprising the expression status of a plurality of the 25 genes in Table 6 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
   (b) inputting the test subject expression profile to a constrained continuation ratio logistic regression model comprising the 4 modifier coefficients (Cp1, Cp2, Cp3 and the intercept) and 25 gene regression coefficients in Table 11, thereby generating 4 risk scores (PUR-1, PUR-2, PUR-3 and PUR-4), wherein the risk scores indicate the likelihood of non-cancerous tissue (PUR-1), low risk of cancer or cancer progression (PUR-2), intermediate-risk of cancer or cancer progression (PUR-3) and high-risk of cancer or cancer progression (PUR-4) in the test subject; and
   (c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.
8. A method of classifying prostate cancer in a test subject or identifying a test subject with a poor prognosis for cancer based on the expression status of a plurality of the genes in Table 2 comprising:
   (a) providing a plurality of patient expression profiles each comprising the expression status of the plurality of genes in at least one sample obtained from each patient, wherein each of the patient expression profiles is associated with one of four cancer risk groups, wherein each of the four cancer risk groups is associated with (i) non-cancerous tissue, (ii) low-risk of cancer or cancer progression, (iii) intermediate-risk of cancer or cancer progression and (iv) high-risk of cancer or cancer progression; optionally wherein each patient expression profile is normalised relative to (i) the expression status of one or more normalising genes in the same patient sample, (ii) an average expression status of one or more normalising genes in a reference population and/or (iii) the status of one or more control-probes;
   (b) applying a cumulative link model to the patient expression profiles to select a subset of one or more genes from the plurality of genes in the patient expression profile that are significantly associated with the four cancer risk groups, optionally wherein the subset of one or more genes is the list of 37 genes in Table 3, the 29 genes in Table 5 or the 25 genes in Table 6;
   (c) inputting the expression values of the selected subset of one or more genes to a constrained continuation ratio logistic regression model comprising three modifier coefficients such that the model generates four risk scores for each patient expression profile, wherein for each patient expression profile, a risk score is provided for each of the four cancer risk groups and wherein each of the four risk scores for a given patient expression profile is associated with the likelihood of membership to the corresponding cancer risk group and wherein the regression model generates regression coefficients associated with each of the selected genes based on the plurality of patient expression profiles;
   (d) providing a test subject expression profile comprising the expression status of the same selected subset of one or more genes as in step (c) in at least one sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
   (e) inputting the test subject expression profile to the constrained continuation ratio logistic regression model comprising the three modifier coefficients and gene regression coefficients generated in step (d) to generate four risk scores (PUR-1, PUR-2, PUR-3 and PUR-4) for the test subject expression profile, wherein each of the four risk scores for the test subject expression profile is associated with the likelihood of membership to the corresponding cancer risk group (i) non-cancerous tissue (PUR-1), (ii) low risk of cancer or cancer progression (PUR-2), (iii) intermediate-risk of cancer or cancer progression (PUR-3) and (iv) high-risk of cancer or cancer progression (PUR-4); and
   (f) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.
9. The method according to embodiments 1 or 2, wherein the plurality of genes in step (a) comprise at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450 or 500 genes.
10. The method according to embodiments 1, 2, 8 or 9, wherein the plurality of genes in step (a) are selected from the genes in Table 2.
11. The method according to any preceding embodiment, wherein the at least one normalising gene is a prostate specific gene (such as those in Table 13) or a constitutively expressed housekeeping gene (such as those in Table 14).
12. The method according to any preceding embodiment, wherein the average expression status of at least one normalising gene in a reference population is the median, mean or modal expression status of the at least one normalising gene in a patient population or population of individuals without prostate cancer (for example a population of at least 50, 100, 200, 300, 400, 500, 600, 700, 800, 900, 1000, 2000, 3000, 4000, 5000, 6000, 7000, 8000, 9000 or 10000 patients or individuals).
13. The method according to any preceding embodiment, wherein the at least one normalising gene is KLK2.
14. The method according to any preceding embodiment, wherein the number of cancer risk groups (n) is 1, 2, 3, 4, 5, 6, 7, 8, 9 or 10.
15. The method according to any preceding embodiment, wherein the n cancer risk groups comprise a group associated with no cancer diagnosis and one or more groups (e.g. 1, 2, 3 groups) associated with increasing risk of cancer diagnosis, severity of cancer or chance of cancer progression.
16. The method according to any preceding embodiment, wherein the higher a risk score is the higher the probability a given patient or test subject exhibits or will exhibit the clinical features or outcome of the corresponding cancer risk group.
17. The method according to any preceding embodiment, wherein at least one of the cancer risk groups is associated with a poor prognosis of cancer.
18. The method according to any preceding embodiment, wherein the number of cancer risk groups (n) is 4.
19. The method according to embodiment 18, wherein the 4 cancer risk groups are the D'Amico risk groups or are equivalent to the D'Amico risk groups (i.e. no evidence of cancer, low-risk of cancer or cancer progression, intermediate-risk of cancer or cancer progression and high-risk of cancer or cancer progression).
20. The method according to embodiments 1 or 2, wherein step (c) further comprises discarding any genes that are not significantly associated with any of the n cancer risk groups.
21. The method according to any preceding embodiment, wherein the test subject expression profile is normalised against the median expression status of KLK2 in a patient population or population of individuals without prostate cancer (for example a population of at least 50, 100, 200, 300, 400, 500, 600, 700, 800, 900, 1000, 2000, 3000, 4000, 5000, 6000, 7000, 8000, 9000 or 10000 patients or individuals).
22. The method according to embodiment 3, wherein the subset of one or more genes is selected from the list of genes in Table 3 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 of the genes in Table 3).
23. The method according to embodiment 3, wherein the subset of one or more genes is selected from the list of genes in Table 4 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 of the genes in Table 4).
24. The method according to embodiment 3, wherein the subset of one or more genes is selected from the list of genes in Table 5 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of the genes in Table 5).
25. The method according to embodiment 3, wherein the subset of one or more genes is selected from the list of genes in Table 6 (i.e. 1, 2, 3, 4, 5, 6, 7, 8, 9, 10, 11, 12, 13, 14, 15, 16, 17, 18, 19, 20, 21, 22, 23, 24 or 25 of the genes in Table 6).
26. The method according to any one of embodiments 4, 5, 6, 7 or 8, wherein a PUR-4 score (high-risk of cancer or cancer progression) of >0.174 indicates a poor prognosis or indicates an increased likelihood of disease progression.
27. A method of diagnosing or testing for prostate cancer comprising determining the expression status of:
   (i) one or more genes selected from the group consisting of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2;
   (ii) one or more genes selected from the group consisting of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2;
   (iii) one or more genes selected from the group consisting of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2; or
   (iv) one or more genes selected from the group consisting of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2,
   in a biological sample.
28. The method according to embodiment 27, wherein the method comprises determining the expression status of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 genes.
29. The method according to embodiment 27 or 28, wherein the method comprises determining the expression status of all 37 genes in embodiment 27(i), all 33 genes in embodiment 27(ii) all 29 genes in embodiment 27(iii) or all 25 genes in embodiment 27(iv).
30. The method according to any preceding embodiment, wherein the method can be used to predict the likelihood of normal tissue, Low-risk, Intermediate-risk, and/or High-risk cancerous tissue being present in the prostate (e.g. based on the D'Amico scale).
31. The method according to any preceding embodiment, wherein the method can be used to determine whether a patient should be biopsied.
32. The method according to embodiment 31, wherein the method is used in combination with MRI imaging data to determine whether a patient should be biopsied.
33. The method according to embodiment 32, wherein the MRI imaging data is generated using multiparametric-MRI (MP-MRI).
34. The method according to any one of embodiments 31 to 33, wherein the MRI imaging data is used to generate a Prostate Imaging Reporting and Data System (PI-RADS) grade.
35. The method according to any preceding embodiment, wherein the method can be used to predict disease progression in a patient.
36. The method according to any preceding embodiment, wherein the patient is currently undergoing or has been recommended for active surveillance.
37. The method according to embodiment 36, wherein the patient is currently undergoing active surveillance by PSA monitoring, biopsy and repeat biopsy and/or MRI, at least every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks or 24 weeks.
38. The method according to any preceding embodiment, wherein the method can be used to predict disease progression in patients with a Gleason score of ≤10, ≤9, ≤8, ≤7 or ≤6.
39. The method according to any preceding embodiment, wherein the method can be used to predict:
   (i) the volume of Gleason ≥4 or Gleason prostate cancer;
   (ii) significant Intermediate- or High-risk disease (based on, for example, the D'Amico grades); and/or
   (iii) low risk disease that will not require treatment for 1, 2, 3, 4, 5 or more years.
40. The method according to any preceding embodiment, wherein the biological sample is processed prior to determining the expression status of the one or more genes in the biological sample.
41. The method according to any preceding embodiment, wherein determining the expression status of the one or more genes comprises extracting RNA from the biological sample.
42. The method of embodiment 41, wherein the RNA extraction step comprises chemical extraction, or solid-phase extraction, or no extraction.
43. The method of embodiment 41, wherein the solid-phase extraction is chromatographic extraction.
44. The method according to any one of embodiments 41 to 43, wherein the RNA is extracted from extracellular vesicles.
45. The method according to any preceding embodiment, wherein determining the expression status of the one or more genes comprises the step of producing one or more cDNA molecules.
46. The method according to any preceding embodiment, wherein determining the expression status of the one or more genes comprises the step of quantifying the expression status of the RNA transcript or cDNA molecule.
47. The method according to embodiment 46 wherein the expression status of the RNA or cDNA is quantified using any one or more of the following techniques: microarray analysis, real-time quantitative PCR, DNA sequencing, RNA sequencing, Northern blot analysis, in situ hybridisation and/or detection and quantification of a binding molecule.
48. The method according to embodiment 46 or 47, wherein the step of quantification of the expression status of the RNA or cDNA comprises RNA or DNA sequencing.
49. The method according to embodiment 46 or 47, wherein the step of quantification of the expression status of the RNA or cDNA comprises using a microarray.
50. The method according to embodiment 49, further comprising the step of capturing the one or more RNAs or cDNAs on a solid support and detecting hybridisation.
51. The method according to embodiment 49 or 50, further comprising sequencing the one or more

RNA or cDNA molecules.
52. The method according to any one of embodiments 49 to 51, wherein the microarray comprises a probe having a nucleotide sequence with at least 80%, 85%, 90%, 95%, 96%, 97%, 98% or 99% identity to a nucleotide sequence selected from any one of SEQ ID NOs 1 to 76.
53. The method according to any one of embodiments 59 to 52, wherein the microarray comprises a probe having a nucleotide sequence selected from any one of SEQ ID NOs 1 to 76.
54. The method according to any one of embodiments 49 to 53, wherein the microarray comprises 74 probes each having a nucleotide sequence with at least 80%, 85%, 90%, 95%, 96%, 97%, 98% or 99% identity to a unique nucleotide sequence selected from any one of SEQ ID NOs 1 to 74.
55. The method according to any one of embodiments 49 to 53, wherein the microarray comprises 74 probes, each having a unique nucleotide sequence selected from SEQ ID NOs 1 to 74.
56. The method according to any one of embodiments 49 to 52, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72 and SEQ ID NOs: 73 and 74.
57. The method according to embodiment 56, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72 and SEQ ID NOs: 73 and 74.
58. The method according to any one of embodiments 49 to 52, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74 and SEQ ID NOs: 75 and 76.
59. The method according to embodiment 58, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74 and SEQ ID NOs: 75 and 76.
60. The method according to any one of embodiments 49 to 52, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74, and SEQ ID NOs: 75 and 76.
61. The method according to embodiment 60, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74, and SEQ ID NOs: 75 and 76.
62. The method according to any one of embodiments 49 to 52, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74 and SEQ ID NOs: 75 and 76.
63. The method according to embodiment 62, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74 and SEQ ID NOs: 75 and 76.
64. The method according to any one of embodiments 49 to 52, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 19 and 20, SEQ ID NOs: 21 and 22, SEQ ID NOs: 23 and 24, SEQ ID NOs: 25 and 26, SEQ ID NOs: 29 and 30, SEQ ID NOs: 39 and 40, SEQ ID NOs: 41 and 42, SEQ ID NOs: 43 and 44, SEQ ID NOs: 51 and 52, SEQ ID NOs: 53 and 54, SEQ ID NOs: 57 and 58, SEQ ID NOs: 59 and 60, SEQ ID NOs: 65 and 66, SEQ ID NOs: 67 and 68, SEQ ID NOs: 69 and 70, SEQ ID NOs: 71 and 72, and SEQ ID NOs: 73 and 74.
65. The method according to embodiment 64, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 19 and 20, SEQ ID NOs: 21 and 22, SEQ ID NOs: 23 and 24, SEQ ID NOs: 25 and 26, SEQ ID NOs: 29 and 30, SEQ ID NOs: 39 and 40, SEQ ID NOs: 41 and 42, SEQ ID NOs: 43 and 44, SEQ ID NOs: 51 and 52, SEQ ID NOs: 53 and 54, SEQ ID NOs: 57 and 58, SEQ ID NOs: 59 and 60, SEQ ID NOs: 65 and 66, SEQ ID NOs: 67 and 68, SEQ ID NOs: 69 and 70, SEQ ID NOs: 71 and 72, and SEQ ID NOs: 73 and 74.
66. The method according to any preceding embodiment, further comprising the step of comparing or normalising the expression status of one or more genes with the expression status of a reference gene.
67. The method according to embodiment 66, wherein the expression status of a reference gene is determined in a biological sample from a healthy patient or one not known to have prostate cancer.
68. The method according to embodiment 67, wherein the expression status of a reference gene is determined in a biological sample from a patient known to have or suspected of having prostate cancer.
69. The method according to embodiment 66 or 67, wherein the expression status of a reference gene is determined in a biological sample from a patient known to have Low-risk, Intermediate-risk, and/or High-risk cancerous tissue (e.g. on the D'Amico scale).
70. The method according to any one of embodiments 66 to 69, wherein the expression status of one or more genes of interest is compared or normalised to KLK2 as a reference gene.
71. The method according to any one of embodiments 66 to 69, wherein the expression status of one or more genes of interest is compared or normalised to KLK3 as a reference gene.
72. The method according to any one of embodiments 66 to 71, wherein the step of comparing or normalising the expression status of one or more genes comprises a loge transformation of the expression status values.
73. The method according to any preceding embodiment wherein the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample).
74. The method according to any preceding embodiment wherein the biological sample is a urine sample.
75. The method according to any preceding embodiment wherein the sample is from a human.
76. The method according to any preceding embodiment, wherein the biological sample is from a patient having or suspected of having prostate cancer.
77. A method of treating prostate cancer, comprising diagnosing a patient as having or as being suspected of having prostate cancer using a method as defined in any one of embodiments 1 to 76, and administering to the patient a therapy for treating prostate cancer.
78. A method of treating prostate cancer in a patient, wherein the patient has been determined as having prostate cancer or as being suspected of having prostate cancer according to a method as defined in any one of embodiments 1 to 76, comprising administering to the patient a therapy for treating prostate cancer.
79. The method according to embodiment 77 or 78, wherein the therapy for prostate cancer comprises active surveillance, chemotherapy, hormone therapy, immunotherapy and/or radiotherapy.
80. The method according to embodiment 79, wherein the chemotherapy comprises administration of one or more agents selected from the following list: abiraterone acetate, apalutamide, bicalutamide, cabazitaxel, bicalutamide, degarelix, docetaxel, leuprolide acetate, enzalutamide, apalutamide, flutamide, goserelin acetate, mitoxantrone, nilutamide, sipuleucel-T, radium 223 dichloride and docetaxel.
81. The method according to embodiment 77 or 78, wherein the therapy for prostate cancer comprises resection of all or part of the prostate gland or resection of a prostate tumour.
82. An RNA or cDNA molecule of one or more genes selected from the group consisting of:
   (i) AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2;
   (ii) AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2;
   (iii) AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2; or
   (iv) AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2,
   for use in a method of diagnosing prostate cancer comprising determining the expression status of the one or more genes.
83. An RNA or cDNA molecule for use according to embodiment 82, wherein the expression status of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 of the genes listed in embodiment 82 is determined.
84. An RNA or cDNA molecule for use according to embodiment 82 or 83, wherein the expression status of all 37 genes in embodiment 82(i), all 33 genes in embodiment 82(ii), all 29 genes in embodiment 82(iii) or all 25 genes in embodiment 92(iv) are determined.
85. An RNA or cDNA molecule for use according to any one of embodiments 82 to 84, wherein expression status of one or more genes can be used to determine whether a patient should be biopsied.
86. An RNA or cDNA molecule for use according to any one of embodiments 82 to 85, wherein expression status of one or more genes can be used to predict disease progression in a patient.
87. An RNA or cDNA molecule for use according to any one of embodiments 82 to 86, wherein the patient is currently undergoing or has been recommended for active surveillance.
88. An RNA or cDNA molecule for use according to embodiment 87, wherein the patient is currently undergoing active surveillance by PSA monitoring, biopsy and repeat biopsy and/or MRI, at least every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks or 24 weeks.
89. An RNA or cDNA molecule for use according to any one of embodiments 82 to 88, wherein the method can be used to predict disease progression patients with a Gleason score of ≤10, ≤9, ≤8, ≤7 or ≤6.
90. An RNA or cDNA molecule for use according to any one of embodiments 82 to 89, wherein the method can be used to predict:
   (i) the volume of Gleason ≥4 or Gleason prostate cancer;
   (ii) significant Intermediate- or High-risk disease (based on, for example, the D'Amico grades); and/or
   (iii) low risk disease that will not require treatment for 1, 2, 3, 4, 5 or more years.
91. A kit for testing for prostate cancer comprising a means for measuring the expression status of:
   (i) one or more genes selected from the group consisting of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2;
   (ii) one or more genes selected from the group consisting of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2;
   (iii) one or more genes selected from the group consisting of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2; or
   (iv) one or more genes selected from the group consisting of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2,
   in a biological sample.
92. The kit according to embodiment 91, comprising a means for measuring the expression status of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 of the genes.
93. The kit according to embodiment 91 or 92, wherein the means for detecting is a biosensor or specific binding molecule.
94. The kit according to any one of embodiments 91 to 93, wherein the biosensor is an electrochemical, electronic, piezoelectric, gravimetric, pyroelectric biosensor, ion channel switch, evanescent wave, surface plasmon resonance or biological biosensor
95. The kit according to any one of embodiments 91 to 94, wherein the means for detecting the expression status of the one or more genes is a microarray.
96. The kit according to embodiment 91, wherein the microarray comprises specific probes that hybridise to one or more of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2.
97. The kit according to embodiment 91, wherein the microarray comprises specific probes that hybridise to one or more of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1 and UPK2.
98. The kit according to embodiment 91, wherein the microarray comprises probes that hybridise to one or more of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2.
99. The kit according to embodiment 91, wherein the microarray comprises probes that hybridise to one or more of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2.
100. The kit according to any one of embodiments 91 to 99, wherein the microarray comprises a probe having a nucleotide sequence with at least 80%, 85%, 90%, 95%, 96%, 97%, 98% or 99% identity to a nucleotide sequence selected from any one of SEQ ID NOs 1 to 76.
101. The kit according to any one of embodiments 91 to 100, wherein the microarray comprises a probe having a nucleotide sequence selected from any one of SEQ ID NOs 1 to 76.
102. The kit according to any one of embodiments 91 to 95, wherein the microarray comprises 74 probes each having a nucleotide sequence with at least 80%, 85%, 90%, 95%, 96%, 97%, 98% or 99% identity to a unique nucleotide sequence selected from any one of SEQ ID NOs 1 to 74.
103. The kit according to any one of embodiments 91 to 95, wherein the microarray comprises 74 probes, each having a unique nucleotide sequence selected from SEQ ID NOs 1 to 74.
104. The kit according to any one of embodiments 91 to 95, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72 and SEQ ID NOs: 73 and 74.
105. The kit according to embodiment 104, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 17 and 18, SEQ ID NOs: 19 and 20, SEQ ID NOs: 21 and 22, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72 and SEQ ID NOs: 73 and 74.
106. The kit according to any one of embodiments 91 to 95, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74 and SEQ ID NOs: 75 and 76.
107. The kit according to embodiment 106, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74 and SEQ ID NOs: 75 and 76.
108. The kit according to any one of embodiments 91 to 95, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74, and SEQ ID NOs: 75 and 76.
109. The kit according to embodiment 108, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74, and SEQ ID NOs: 75 and 76.
110. The kit according to any one of embodiments 91 to 95, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74 and SEQ ID NOs: 75 and 76.
111. The kit according to embodiment 110, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 17 and 18, SEQ ID NOs: 19 and 20, SEQ ID NOs: 21 and 22, SEQ ID NOs: 25 and 26, SEQ ID NOs: 27 and 28, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 51 and 52, SEQ ID NOs: 53 and 54, SEQ ID NOs: 55 and 56, SEQ ID NOs: 57 and 58, SEQ ID NOs: 61 and 62, SEQ ID NOs: 63 and 64, SEQ ID NOs: 65 and 66, SEQ ID NOs: 67 and 68, SEQ ID NOs: 73 and 74 and SEQ ID NOs: 75 and 76.
112. The kit according to any one of embodiments 91 to 95, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 19 and 20, SEQ ID NOs: 21 and 22, SEQ ID NOs: 23 and 24, SEQ ID NOs: 25 and 26, SEQ ID NOs: 29 and 30, SEQ ID NOs: 39 and 40, SEQ ID NOs: 41 and 42, SEQ ID NOs: 43 and 44, SEQ ID NOs: 51 and 52, SEQ ID NOs: 53 and 54, SEQ ID NOs: 57 and 58, SEQ ID NOs: 59 and 60, SEQ ID NOs: 65 and 66, SEQ ID NOs: 67 and 68, SEQ ID NOs: 69 and 70, SEQ ID NOs: 71 and 72, and SEQ ID NOs: 73 and 74.
113. The kit according to embodiment 112, wherein the microarray comprises a pair of probes for every gene of interest having nucleotide sequences selected from the following list: SEQ ID NOs: 1 and 2, SEQ ID NOs: 3 and 4, SEQ ID NOs: 5 and 6, SEQ ID NOs: 7 and 8, SEQ ID NOs: 9 and 10, SEQ ID NOs: 11 and 12, SEQ ID NOs: 13 and 14, SEQ ID NOs: 15 and 16, SEQ ID NOs: 19 and 20, SEQ ID NOs: 21 and 22, SEQ ID NOs: 23 and 24, SEQ ID NOs: 25 and 26, SEQ ID NOs: 29 and 30, SEQ ID NOs: 39 and 40, SEQ ID NOs: 41 and 42, SEQ ID NOs: 43 and 44, SEQ ID NOs: 51 and 52, SEQ ID NOs: 53 and 54, SEQ ID NOs: 57 and 58, SEQ ID NOs: 59 and 60, SEQ ID NOs: 65 and 66, SEQ ID NOs: 67 and 68, SEQ ID NOs: 69 and 70, SEQ ID NOs: 71 and 72, and SEQ ID NOs: 73 and 74.
114. The kit according to any one of embodiments 91 to 113, wherein the kit further comprises one or more solvents for extracting RNA from the biological sample.
115. A computer apparatus configured to perform a method according to any one of embodiments 1 to 76.
116. A computer readable medium programmed to perform a method according to any one of embodiments 1 to 76.
117. A kit of any one of embodiments 91 to 113, further comprising a computer readable medium as defined in embodiment 116.

### REFERENCES

[1] D'Amico A V., Moul J, Carroll P R, Sun L, Lubeck D, Chen M H. Cancer-specific mortality after surgery or radiation for patients with clinically localized prostate cancer managed during the prostate-specific antigen era. J Clin Oncol. 2003; 21(11):2163-2172. doi:10.1200/JCO.2003.01.075.
[2] D'Amico A V., Whittington R, Bruce Malkowicz S, et al. Biochemical outcome after radical prostatectomy, external beam radiation therapy, or interstitial radiation therapy for clinically localized prostate cancer. J Am Med Assoc. 1998; 280(11):969-974. doi:10.1001/jama.280.11.969.
[3] Epstein J I, Zelefsky M J, Sjoberg D D, et al. A Contemporary Prostate Cancer Grading System: A Validated Alternative to the Gleason Score. Eur Urol. 2016; 69(3):428-435. doi:10.1016/j.eururo.2015.06.046.
[4] Sanda M G, Cadeddu J A, Kirkby E, et al. Clinically Localized Prostate Cancer: AUA/ASTRO/SUO Guideline. Part I: Risk Stratification, Shared Decision Making, and Care Options. J Urol. 2018; 199(3):683-690. doi:10.1016/j.juro.2017.11.095.
[5] Mottet N, Bellmunt J, Bolla M, et al. EAU-ESTRO-SIOG Guidelines on Prostate Cancer. Part 1: Screening, Diagnosis, and Local Treatment with Curative Intent. Eur Urol. 2017; 71(4):618-629. doi:10.1016/j.eururo.2016.08.003.
[6] National Institute for Health and Care Excellence. Prostate Cancer: Diagnosis and Treatment.; 2014.
[7] Selvadurai E D, Singhera M, Thomas K, et al. Medium-term outcomes of active surveillance for localised prostate cancer. Eur Urol. 2013; 64(6):981-987. doi:10.1016/j.eururo.2013.02.020.
[8] Cooperberg M R, Freedland S J, Pasta D J, et al. Multiinstitutional validation of the UCSF cancer of the prostate risk assessment for prediction of recurrence after radical prostatectomy. Cancer. 2006; 107(10):2384-2391. doi:10.1002/cncr.22262.
[9] Brajtbord J S, Leapman M S, Cooperberg M R. The CAPRA Score at 10 Years: Contemporary Perspectives and Analysis of Supporting Studies. Eur Urol. 2017; 71(5):705-709. doi:10.1016/j.eururo.2016.08.065.
[10] Flier J S, Underhill L H, Zetter B R. The Cellular Basis of Site-Specific Tumour Metastasis. N Engl J Med. 1990 March; 322(9):605-12.
[11] Gleason D F. Histologic grading of prostate cancer: A perspective. Human Pathology. 1992 March; 23(3):273-9.
[12] Montironi R, Mazzuccheli R, Scarpelli M, Lopez-Beltran A, Fellegara G, Algaba F. Gleason grading of prostate cancer in needle biopsies or radical prostatectomy specimens: contemporary approach, current clinical significance and sources of pathology discrepancies. BJU Int. 2005 June; 95(8):1146-52.
[13] Villers A, McNeal J E, Redwine E A, Freiha F S, Stamey T A. The role of perineural space invasion in the local spread of prostatic adenocarcinoma. JURO. 1989 Sep. 1; 142(3):763-8.
[14] Epstein J I. Epstein: Pathology of adenocarcinoma of the prostate. Campbell's Urology. 1998.
[15] Andreoiu M, Cheng L. Multifocal prostate cancer: biologic, prognostic, and therapeutic implications. Hum Pathol. 2010; 41(6):781-793. doi:10.1016/j.humpath.2010.02.011.
[16] Corcoran N M, Hovens C M, Hong M K H, et al. Underestimation of Gleason score at prostate biopsy reflects sampling error in lower volume tumours. BJU Int. 2012; 109(5):660-664. doi:10.1111/j.1464-410X.2011.10543.x.
[17] Ahmed H U, EI-Shater Bosaily A, Brown L C, et al. Diagnostic accuracy of multi-parametric MRI and TRUS biopsy in prostate cancer (PROMIS): a paired validating confirmatory study. Lancet. 2017; 389(10071):815-822. doi:10.1016/50140-6736(16)32401-1.
[18] Tomlins S A, Day J R, Lonigro R J, et al. Urine TMPRSS2:ERG Plus PCA3 for Individualized Prostate Cancer Risk Assessment. Eur Urol. 2016; 70(1):45-53. doi:10.1016/j.eururo.2015.04.039.
[19] McKiernan J, Donovan M J, O'Neill V, et al. A novel urine exosome gene expression assay to predict high-grade prostate cancer at initial biopsy. JAMA Oncol. 2016; 2(7):882-889. doi:10.1001/jamaonco1.2016.0097.
[20] Donovan M J, Noerholm M, Bentink S, et al, A molecular signature of PCA3 and ERG exosomal RNA from non-DRE urine is predictive of initial prostate biopsy result. Pmstate Cancer Prostatic Dis. 2015:18(4):370-375. doi:10.1038/pcan.2015.40.
[21] Van Neste L. Hendriks R J. Dijkstra S. et al. Detection of High-grade Prostate Cancer Using a Urinary Molecular Biomarker-Based Risk Score. Eur Urol. 2016:70(5):740-748. doi:10.1016/j.eururo.2016.04.012.
[22] Ilic D, O'Connor D, Green S, Wilt T, Screening for prostate cancer. Cochrane Database Syst Rev. 2006:(3):CD004720.
[23] Screening for Prostate Cancer: A Review of the Evidence for the U.S. Preventive Services Task Force. 2011 Nov 17;:1-22.
[24] Schröder, FH et al., Screening and prostate cancer mortality: results of the European Randomised Study of Screening for Prostate Cancer (ERSPC) at 13 years of follow-up. Lancet. 2014 Dec 6:384(9959):2027-35.
[25] lemaitre L, Puech P, Poncelet E, Bouy6 S, Leroy X, Biserte J, et al, Dynamic contrast-enhanced MRI of anterior prostate cancer: morphometric assessment and correlation with radical prostatectomy findings. Eur Radiol. 2009 Feb 1:19(2):470-80.
[26] Bouyé S, Potiron E, Puech P, Leroy X, Lemaitre L, Villers A, Transition zone and anterior stromal prostate cancers: zone of origin and intraprostatic patterns of spread at histopathology. Prostate. 2009 Jan 1;69(1):105-13.
[27] Scattoni V, Zlotta A, Montironi R, Schulman C, Rigatti P, Montorsi F, Extended and Saturation Prostatic Biopsy in the Diagnosis and Characterisation of Prostate Cancer A Critical Analysis of the Literature. European Urology. 2007 Jan 1:52(5): 1309-22.
[28] Luca et al., DESNT: A Poor Prognosis Category of Human Prostate Cancer. Eur Urol Focus. 2017 Mar 6. pii: S2405-4569(17)30025-1.
[29] Hessels. D. et al. DD3PCA3-based molecular urine analysis for the diagnosis of prostate cancer. Eur. Urol. 44, 6-16 (2003)
[30] Bologna, M. et al. Early diagnosis of prostatic carcinoma based on in vitro culture of viable tumor cells harvested by prostatic massage. Eur. Urol. 14. 474-476 (1988).
[31] Garret. M. & Jassie. M Cytologic examination of post prostatic massage specimens as an aid in diagnosis of carcinoma of the prostate. Acta Cytol. 20. 126-31
[32] Rak J. Microparticles in cancer. Semin Thromb Hemost 2010 Nov:36(8):888-906
[33] Mathivanan S, Ji H, Simpson R J. Exosomes: Extracellular organelles important in intercellular communication. Journal of Proteomics. Elsevier B. V: 2010 Sep 10:73(10):1907-20.
[34] van der Pol E. Bomg A N, Harrison P, Sturk A, Nieuwland R, Classification. Functions, and Clinical Relevance of Extracellular Vesicles. Pharmacological Reviews. 2012 Jul 2;64(3):676-705.
[35] Keller S, Sanderson MP, Stoeck A, Altevogt P, Exosomes: from biogenesis and secretion to biological function. Immunol Lett 2006 Nov 15:107(2):102-8.
[36] Simons M. Raposo G. Exosomes-vesicular carriers for intercellular communication. Current Opinion in Cell Biology. 2009 Aug:21(4):575-81.
[37] van Niel G. Exosomes: A Common Pathway for a Specialized Function. Journal of Biochemistry. 2006 Jul 1;140(1):13-21.
[38] Mears R, Craven R A, Hanrahan S. Totty N, Proteomic analysis of melanoma-denved exosomes by two dimensional polyacrylamide gel electrophoresis and mass spectrometry. Proteomics 2004 Dec;4(12):4019-31.
[39] Futter C E, White I J, Annexms and endocytosis. Traffic 2007 Aug;8(8):951-8.
[40] Xiao D, Ohlendorf J, Chen Y, Taylor D D. Rai S N, Waigel S, et al, Identifying mRNA, microRNA and protein profiles of melanoma exosomes. PLoS ONE. 2012:7(10):e46874.
[41] Wieckowski E, Whiteside T L. Human tumour-derived vs dendritic cell-derived exosomes have distinct biologic roles and molecular profiles. Immunol Res 2006:36(1-3):247-54
[42] Castellana D, Zobairi F, Martinez M C, Panaro M A, Mitolo V, Freyssinet J-M, et al. Membrane microvestcles as actors in the establishment of a favorable prostatic tumoural niche: a role for activated fibroblasts and CX3CL1-CX3CR1 axis. Cancer Research. 2009 Feb 1:69(3):785-93.
[43] Mitchell P J, Welton J, Staffurth J, Court J, Mason M D, Tabi Z, et al, Can urinary exosomes act as treatment response markers In prostate cancer? J Transl Med 2009:7(1):4.
[44] Schostak M. Schwall G P, Poznanovid S, Groebe K, Müller M. Messinger D. et al. Annexin A3 in Urine: A Highly Specific Nonmvasive Marker for Prostate Cancer Early Detection. The Journal of Urology 2009 Jan;181(1):343-53
[45] Nilsson J, Skog J, Nordstrand A, Baranov V, Mincheva-Nilsson L, 8reakefield X O, et al Prostate cancer-derived urine exosomes: a novel approach to biomarkers for prostate cancer Nature Publishing Group; 2009 Apr 28:100(10):1603-7.
[46] Fitzwater & Polisky (1996) Methods Enzymol, 267:275-301
[47] Christensen RHB (2018). “ordinal-Regression Models for Ordinal Data.” package version 2018.8-25, URL http://Www.cran.r-project.org/package=ordinal/
[48] https://cran.r-project.orgAveb,packages/ordmal/vignettes/clm_aiticJe.pdf
[49] Epstein J I, Allsbrook W C Jr, Amin M B, Egevad L L: ISUP Grading Committee. The 2005 International Society of Urological Pathology (ISUP) Consensus Conference on Gleason grading of prostatic carcinoma. Am J Surg Pathol 2005:29(9): 1228-42
[50] Zhang. G. & Pradhan. S. Mammalian epigenetic mechanisms. IUBMB life (2014).
[51] Gnanbuak, K. at al. A critical appraisal of tools availabla for monitoring opiganotic changus in clinical samplos from patients with myeloid malignancies. Haematologica 97, 1380-1388 (2012).
[52] Ulahannan. N. & Grealty. J. M. Genome-wide assays that identify and quantify modified cytosines in human disease studies. Epigenetics Chromatin 8. 5 (2015).
[53] Crutchley. J. L., Wang, X., Ferraiuolo. M. A. & Dostie, J. Chromatin conformation signatures: ideal human disease biomarkers? Biomarkers (2010).
[54] Esteller. M. Cancer epigenomics: DNA methylomes and histone-modification maps. Nat Rev. Genet. 8, 286-298 (2007).
[55] Deantom E P, Crawford E D, Oesteriing J E, et al. Age- and race-specific reference ranges for prostate-specific antigen from a large community-based study Urology. 1996:48(2)234-239 doi:10.1016/S0090-4295(96)00091-X.
[56] Miranda K C, Bond D T, McKee M. et al. Nucleic acids within urinary exosomes/microvesicles are potential biomarkers for renal disease. Kidney Int. 2010:78(2):191-199 doi:10.1038/ki.2010.106.
[57] (57) Geiss G K, Bumgarner R E, Birditt B, et al. Direct multiplexed measurement of gene expression with color-coded probe pairs. Nat Biotechnol. 2008:26(3):317-325. doi:10 1038/nbt1385.
[58] Johnson W E, Li C. Rabinovic A Adjusting batch effeds in microarray expression data using empirical Bayes methods. Biostatistics. 2007:8(1):118-127. doi:10.1093/biostatetics/kxj037.
[59] https://www.r-project.org/
[60] Archer K J,. Williams A A A, L1 penalized continuation ratio models for ordinal response prediction using high-dimensional datasets. Stat Med. 2012:31(14):1464-1474. doi:10.1002/sim.4484.
[61] Tibshirani R. Regression Shnnkage and Selection via the Lasso. J R Stat Soc Ser B. 1996:58:267-288. doi:10 2307/2346178
[62] Christensen. R. H. B. ordinal-Regression Models for Ordinal Data (2018).
[63] Brown, M. rmda: Risk Model Decision Analysis. (2017).
[64] Martin R M, Donovan J L, Turner E L, et al. Effect of a Low-Intensity PSA-Based Screening Intervention on Prostate Cancer Mortality. JAMA. 2018; 319(9):883. doi:10.1001/jama.2018.0154.
[65] Robin, X. et al. pROC: an open-source package for R and S+to analyze and compare ROC curves. BMC Bioinformatics 12, 77 (2011).
[66] Vickers A J, Elkin E B. Decision Curve Analysis: A Novel Method for Evaluating Prediction Models. Med Decis Mak. 2006; 26(6):565-574. doi:10.1177/0272989X06295361.
[67] Am J Surg Pathol 2005; 29:1228; reviewed, J Urol 2010; 183:433
[68] Vis A N, Roemeling S, Kranse R, Schroder F H, van der Kwast T H. Eur Urol. 2007 April; 51(4):931-9.
[69] Sauter G, et al. Eur Urol. 2016 April; 69(4):592-598. doi: 10.1016/j.eururo.2015.10.029.

1. A method of providing a cancer diagnosis or prognosis based on the expression status of a plurality of genes comprising:
(a) providing a plurality of patient expression profiles each comprising the expression status of the plurality of genes in at least one sample obtained from each patient, wherein each of the patient expression profiles is associated with one or more cancer risk groups, wherein each cancer risk group is associated with a different cancer prognosis or cancer diagnosis, optionally wherein each patient expression profile is normalised relative to (i) the expression status of one or more normalising genes in the same patient sample, (ii) an average expression status of one or more normalising genes in a reference population and/or (iii) the status of one or more control-probes;
(b) counting the number (n) of different cancer risk groups to which the patient expression profiles belong, optionally wherein at least one cancer risk group is associated with an absence of cancer;
(c) applying a cumulative link model to the patient expression profiles to select a subset of one or more genes from the plurality of genes in the patient expression profile that are significantly associated with the n cancer risk groups; and
(d) inputting the expression values of the selected subset of one or more genes to a constrained continuation ratio logistic regression model comprising n modifier coefficients such that the model generates n risk scores for each patient expression profile, wherein for each patient expression profile, a risk score is provided for each of the n cancer risk groups and wherein each of the n risk scores for a given patient expression profile is associated with the likelihood of membership to the corresponding cancer risk group, optionally wherein the regression model generates regression coefficients associated with each of the selected subset of genes based on the plurality of patient expression profiles.

2. A method of classifying prostate cancer in a test subject or identifying a test subject with a poor prognosis for cancer based on the expression status of a plurality of genes comprising:
(a) providing a plurality of patient expression profiles each comprising the expression status of the plurality of genes in at least one sample obtained from each patient, wherein each of the patient expression profiles is associated with one or more cancer risk groups, wherein each cancer risk group is associated with a different cancer prognosis or cancer diagnosis, optionally wherein each patient expression profile is normalised relative to (i) the expression status of one or more normalising genes in the same patient sample, (ii) an average expression status of one or more normalising genes in a reference population and/or (iii) the status of one or more control-probes;
(b) counting the number (n) of different cancer risk groups to which the patient expression profiles belong, optionally wherein at least one cancer risk group is associated with an absence of cancer;
(c) applying a cumulative link model to the patient expression profiles to select a subset of one or more genes from the plurality of genes in the patient expression profile that are significantly associated with the n cancer risk groups;
(d) inputting the expression values of the selected subset of one or more genes to a constrained continuation ratio logistic regression model comprising n modifier coefficients such that the model generates n risk scores for each patient expression profile, wherein for each patient expression profile, a risk score is provided for each of the n cancer risk groups and wherein each of the n risk scores for a given patient expression profile is associated with the clinical outcome of the corresponding cancer risk group and wherein the regression model generates regression coefficients associated with each of the selected genes based on the plurality of patient expression profiles;
(e) providing a test subject expression profile comprising the expression status of the same selected subset of one or more genes as in step (c) in at least one sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
(f) inputting the test subject expression profile to the constrained continuation ratio logistic regression model comprising the n modifier coefficients and gene regression coefficients generated in step (d) to generate n risk scores for the test subject expression profile, wherein each of the n risk scores for the test subject expression profile is associated with the likelihood of membership to the corresponding cancer risk group; and
(g) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

3. A method of classifying prostate cancer in a test subject or identifying a test subject with a poor prognosis for cancer comprising:
(a) providing a test subject expression profile comprising the expression status of a subset of one or more genes selected by a method according to the first aspect of the invention in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
(b) inputting the test subject expression profile to a constrained continuation ratio logistic regression model comprising the n modifier coefficients and gene regression coefficients generated using a method according to the first aspect of the invention, thereby generating n risk scores, wherein each of the n risk scores for a given test subject expression profile is associated with the likelihood of membership to the corresponding cancer risk group, wherein the n modifier coefficients and corresponding gene regression coefficients are generated by applying the regression model to patient expression profiles comprising the expression status of the same subset of one or more genes; and
(c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

4. A method of classifying prostate cancer in a test subject or identifying a test subject with a poor prognosis for cancer comprising:
(a) providing a test subject expression profile comprising the expression status of a plurality of the 37 genes in Table 3 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
(b) inputting the test subject expression profile to a constrained continuation ratio logistic regression model comprising the 4 modifier coefficients (Cp1, Cp2, Cp3 and the intercept) and 36 gene regression coefficients in Table 8, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
(c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

5. A method of classifying prostate cancer in a test subject or identifying a test subject with a poor prognosis for cancer comprising:
(a) providing a test subject expression profile comprising the expression status of a plurality of the 33 genes in Table 4 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
(b) inputting the test subject expression profile to a constrained continuation ratio logistic regression model comprising the 4 modifier coefficients (Cp1, Cp2, Cp3 and the intercept) and 33 gene regression coefficients in Table 9, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
(c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

6. A method of classifying prostate cancer in a test subject or identifying a test subject with a poor prognosis for cancer comprising:
(a) providing a test subject expression profile comprising the expression status of a plurality of the 29 genes in Table 5 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
(b) inputting the test subject expression profile to a constrained continuation ratio logistic regression model comprising the 4 modifier coefficients (Cp1, Cp2, Cp3 and the intercept) and 29 gene regression coefficients in Table 10, thereby generating 4 risk scores (PUR-1, PUR-2, PUR-3 and PUR-4), wherein the risk scores indicate the likelihood of non-cancerous tissue (PUR-1), low-risk of cancer or cancer progression (PUR-2), intermediate-risk of cancer or cancer progression (PUR-3) and high-risk of cancer or cancer progression (PUR-4) in the test subject; and
(c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

7. A method of classifying prostate cancer in a test subject or identifying a test subject with a poor prognosis for cancer comprising:
(a) providing a test subject expression profile comprising the expression status of a plurality of the 25 genes in Table 6 in a sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
(b) inputting the test subject expression profile to a constrained continuation ratio logistic regression model comprising the 4 modifier coefficients (Cp1, Cp2, Cp3 and the intercept) and 25 gene regression coefficients in Table 11, thereby generating 4 risk scores (PUR-1, PUR-2, PUR-3 and PUR-4), wherein the risk scores indicate the likelihood of non-cancerous tissue (PUR-1), low risk of cancer or cancer progression (PUR-2), intermediate-risk of cancer or cancer progression (PUR-3) and high-risk of cancer or cancer progression (PUR-4) in the test subject; and
(c) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

8. A method of classifying prostate cancer in a test subject or identifying a test subject with a poor prognosis for cancer based on the expression status of a plurality of the genes in Table 2 comprising:
(a) providing a plurality of patient expression profiles each comprising the expression status of the plurality of genes in at least one sample obtained from each patient, wherein each of the patient expression profiles is associated with one of four cancer risk groups, wherein each of the four cancer risk groups is associated with (i) non-cancerous tissue, (ii) low-risk of cancer or cancer progression, (iii) intermediate-risk of cancer or cancer progression and (iv) high-risk of cancer or cancer progression; optionally wherein each patient expression profile is normalised relative to (i) the expression status of one or more normalising genes in the same patient sample, (ii) an average expression status of one or more normalising genes in a reference population and/or (iii) the status of one or more control-probes;
(b) applying a cumulative link model to the patient expression profiles to select a subset of one or more genes from the plurality of genes in the patient expression profile that are significantly associated with the four cancer risk groups, optionally wherein the subset of one or more genes is the list of 37 genes in Table 3, the 29 genes in Table 5 or the 25 genes in Table 6;
(c) inputting the expression values of the selected subset of one or more genes to a constrained continuation ratio logistic regression model comprising three modifier coefficients such that the model generates four risk scores for each patient expression profile, wherein for each patient expression profile, a risk score is provided for each of the four cancer risk groups and wherein each of the four risk scores for a given patient expression profile is associated with the likelihood of membership to the corresponding cancer risk group and wherein the regression model generates regression coefficients associated with each of the selected genes based on the plurality of patient expression profiles;
(d) providing a test subject expression profile comprising the expression status of the same selected subset of one or more genes as in step (c) in at least one sample obtained from the test subject, optionally wherein the test subject expression profile is normalised relative to (i) the expression status of one or more normalising genes in the test subject sample, (ii) an average expression status of one or more normalising genes in a reference population, and/or (iii) the status of one or more control-probes;
(e) inputting the test subject expression profile to the constrained continuation ratio logistic regression model comprising the three modifier coefficients and gene regression coefficients generated in step (d) to generate four risk scores (PUR-1, PUR-2, PUR-3 and PUR-4) for the test subject expression profile, wherein each of the four risk scores for the test subject expression profile is associated with the likelihood of membership to the corresponding cancer risk group (i) non-cancerous tissue (PUR-1), (ii) low risk of cancer or cancer progression (PUR-2), (iii) intermediate-risk of cancer or cancer progression (PUR-3) and (iv) high-risk of cancer or cancer progression (PUR-4); and
(f) classifying the cancer of the test subject or determining whether the test subject has a poor prognosis based on the value of a risk score associated with a poor prognosis cancer risk group for the test subject expression profile, wherein the higher the risk score associated with a poor prognosis cancer risk group, the worse the predicted outcome.

9. The method according to claim 1 or 2, wherein the plurality of genes in step (a) comprise at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450 or 500 genes.

10. The method according to claim 1, 2, 8 or 9, wherein the plurality of genes in step (a) are selected from the genes in Table 2.

11. The method according to any preceding claim, wherein the n cancer risk groups comprise a group associated with no cancer diagnosis and one or more groups (e.g. 1, 2, 3 groups) associated with increasing risk of cancer diagnosis, severity of cancer or chance of cancer progression.

12. The method according to any preceding claim, wherein the higher a risk score is the higher the probability a given patient or test subject exhibits or will exhibit the clinical features or outcome of the corresponding cancer risk group.

13. The method according to claim 11, wherein n=4 and wherein the 4 cancer risk groups are the D'Amico risk groups or are equivalent to the D'Amico risk groups (i.e. no evidence of cancer, low-risk of cancer or cancer progression, intermediate-risk of cancer or cancer progression and high-risk of cancer or cancer progression).

14. The method according to claim 3, wherein the subset of one or more genes is selected from the list of genes in Table 3 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 of the genes in Table 3).

15. A method of diagnosing or testing for prostate cancer comprising determining the expression status of:
(i) one or more genes selected from the group consisting of AMACR, AMH, ANKRD34B, APOC1, AR (exons 4-8), DPP4, ERG (exons 4-5), GABARAPL2, GAPDH, GDF15, HOXC6, HPN, IGFBP3, IMPDH2, ITGBL1, KLK2, KLK4, MARCH5, MED4, MEMO1, MEX3A, MME, MMP11, MMP26, NKAIN1, PALM3, PCA3, PPFIA2, SIM2-short, SMIM1, SSPO, SULT1A1, TDRD1, TMPRSS2:ERG, TRPM4, TWIST1 and UPK2;
(ii) one or more genes selected from the group consisting of AMACR, AMH, ANKRD34B, APOC1, ARexons4-8, CD10, DPP4, GABARAPL2, GAPDH, HOXC6, HPN, IGFBP3, IMPDH2, ITGBL1, KLK4, MED4, MEMO1, MEX3A, MIC1, MMP26, NKAIN1, PALM3, PCA3, PPFIA2, SIM2.short, SMIM1, SSPO, SULT1A1, TDRD, TMPRSS2/ERG fusion, TRPM4, TWIST1, UPK2;
(iii) one or more genes selected from the group consisting of AMACR, AMH, ANKRD34B, APOC1, AR (exons 4-8), CD10, DPP4, GAPDH, HOXC6, IGFBP3, IMPDH2, KLK2, KLK4, MARCH5, MED4, MEMO1, MEX3A, MIC1, MMP11, MMP26, PALM3, PCA3, PPFIA2, SIM2-short, SLC12A1, SSPO, SULT1A1, TDRD, TMPRSS2:ERG and UPK2; or
(iv) one or more genes selected from the group consisting of AMACR, AMH, ANKRD34B, APOC1, ARexons4-8, CD10, DPP4, ERG 3 ex 4-5, GABARAPL2, HOXC6, HPN, IGFBP3, ITGBL1, MEMO1, MEX3A, MIC1, PALM3, PCA3, SIM2.short, SMIM1, TDRD, TMPRSS2:ERG, TRPM4, TWIST1 and UPK2,
in a biological sample.

16. The method according to any preceding claim, wherein the method can be used to predict the likelihood of normal tissue, Low-risk, Intermediate-risk, and/or High-risk cancerous tissue being present in the prostate (e.g. based on the D'Amico scale).

17. The method according to any preceding claim, wherein the method can be used to determine whether a patient should be biopsied.

18. The method according to any preceding claim, wherein the method can be used to predict disease progression in a patient.

19. The method according to any preceding claim, wherein the patient is currently undergoing or has been recommended for active surveillance.

20. The method according to any preceding claim, wherein the method can be used to predict:
(i) the volume of Gleason 4 or Gleason ≥4 prostate cancer;
(ii) significant Intermediate- or High-risk disease (based on, for example, the D'Amico grades); and/or
(iii) low risk disease that will not require treatment for 1, 2, 3, 4, 5 or more years.

21. The method according to any preceding claim, wherein determining the expression status of the one or more genes comprises extracting RNA from the biological sample.

22. The method according to claim 21, wherein the RNA is extracted from extracellular vesicles.

23. The method according to any preceding claim wherein determining the expression status of the one or more genes comprises the step of quantifying the expression status of the RNA transcript or cDNA molecule and wherein the expression status of the RNA or cDNA is quantified using any one or more of the following techniques: microarray analysis, real-time quantitative PCR, DNA sequencing, RNA sequencing, Northern blot analysis, in situ hybridisation and/or detection and quantification of a binding molecule.

24. The method according to any preceding claim, further comprising the step of comparing or normalising the expression status of one or more genes with the expression status of a reference gene.

25. The method according to any preceding claim wherein the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample).


SEQUENCE LISTING (76 sequences)

SEQ ID NO: 1 - DNA, 50 bp, Artificial Sequence
  Capture probe
tggaatctaccccttcctcacatgcctttaggaagttgagtccagggaag

SEQ ID NO: 2 - DNA, 50 bp, Artificial Sequence
  Reporter probe
caacatccattctctactccctctactctgatggcacccggattagattg

SEQ ID NO: 3 - DNA, 35 bp, Artificial Sequence
  Capture probe
ttggcctggtaggtctcggggatgagtacggagcg

SEQ ID NO: 4 - DNA, 35 bp, Artificial Sequence
  Reporter probe
cggactgaggccagccgcacacgccctggcaattg

SEQ ID NO: 5 - DNA, 50 bp, Artificial Sequence
  Capture probe
tttataggatagttcttcctctggtgtaatatcctggagctcctcttgca

SEQ ID NO: 6 - DNA, 50 bp, Artificial Sequence
  Reporter probe
atgctttggtgcctagtgatgaaccgcttggaaagtgccagcccattggt

SEQ ID NO: 7 - DNA, 46 bp, Artificial Sequence
  Capture probe
cggaggggcactctgaatccttgctggagggcttggttgggaggtc

SEQ ID NO: 8 - DNA, 50 bp, Artificial Sequence
  Reporter probe
cagaaccaccaccaggaccgggagcgacaggaagagcctcatggcgaggc

SEQ ID NO: 9 - DNA, 50 bp, Artificial Sequence
  Capture probe
gacttgtgcatgcggtactcattgaaaaccagatcaggggcgaagtagag

SEQ ID NO: 10 - DNA, 50 bp, Artificial Sequence
  Reporter probe
caaactcttgagagaggtgcctcattcggacacactggctgtacatccgg

SEQ ID NO: 11 - DNA, 50 bp, Artificial Sequence
  Capture probe
aaatccactccaacatcgaccagggctttggagatctgagctgactgctg

SEQ ID NO: 12 - DNA, 45 bp, Artificial Sequence
  Reporter probe
ctgctagctattccatggtcttcatcagtataccacattgcctgg

SEQ ID NO: 13 - DNA, 50 bp, Artificial Sequence
  Capture probe
tgagccattcacctggctagggttacattccattttgatggtgaccctgg

SEQ ID NO: 14 - DNA, 49 bp, Artificial Sequence
  Reporter probe
ccaccatcttcccgcctttggccacactgcattcatcaggagagttcct

SEQ ID NO: 15 - DNA, 50 bp, Artificial Sequence
  Capture probe
gggactgtcttatccacaaacaggaagatcgccttttcagaaggaagctg

SEQ ID NO: 16 - DNA, 50 bp, Artificial Sequence
  Reporter probe
cttcatctttttccttctcgtaaagctgtcccatagttaggctggactgt

SEQ ID NO: 17 - DNA, 39 bp, Artificial Sequence
  Capture probe
aagtggtcgttgagggcaatgccagccccagcgtcaaag

SEQ ID NO: 18 - DNA, 50 bp, Artificial Sequence
  Reporter probe
ccctgttgctgtagccaaattcgttgtcataccaggaaatgagcttgaca

SEQ ID NO: 19 - DNA, 36 bp, Artificial Sequence
  Capture probe
cctggttagcaggtcctcgtagcgtttccgcaactc

SEQ ID NO: 20 - DNA, 35 bp, Artificial Sequence
  Reporter probe
gtgttcgaatcttcccagctctggttggcccgcag

SEQ ID NO: 21 - DNA, 50 bp, Artificial Sequence
  Capture probe
ggtcgagaaatgcctcactggatcataggcggtggaattgagggcgacgt

SEQ ID NO: 22 - DNA, 50 bp, Artificial Sequence
  Reporter probe
gaataaaagggagtcgagtagatccggttctgggcaacggccgctccata

SEQ ID NO: 23 - DNA, 40 bp, Artificial Sequence
  Capture probe
ccgagagatgctgtcctcacacacaaagggaccaccgctg

SEQ ID NO: 24 - DNA, 36 bp, Artificial Sequence
  Reporter probe
ccaactcacaatgccacacagccgccaacgtggcgt

SEQ ID NO: 25 - DNA, 50 bp, Artificial Sequence
  Capture probe
cgggcgcatgaagtctgggtgctgtgctcgagtctctgaatattttgata

SEQ ID NO: 26 - DNA, 38 bp, Artificial Sequence
  Reporter probe
tggtcggccgcttcgaccaacatgtggtgagcattcca

SEQ ID NO: 27 - DNA, 50 bp, Artificial Sequence
  Capture probe
tctttgagaaaatcaatgtccctggaggagatgatgcccaccaagcggct

SEQ ID NO: 28 - DNA, 45 bp, Artificial Sequence
  Reporter probe
tccctctttgtcattatctcttccaagaaacagtcatgttcctcc

SEQ ID NO: 29 - DNA, 50 bp, Artificial Sequence
  Capture probe
agaccacaccatcgaggtcttcacagcggcgatcatcacactcacaagtc

SEQ ID NO: 30 - DNA, 50 bp, Artificial Sequence
  Reporter probe
tcctctctcacaaacacagcgaccacaggaacatgtgccgtggcctccac

SEQ ID NO: 31 - DNA, 50 bp, Artificial Sequence
  Capture probe
cttggacactaaggatcaggtgagcttcctcagttggaattactttgtac

SEQ ID NO: 32 - DNA, 50 bp, Artificial Sequence
  Reporter probe
gtcaattattcaagtactccatactcgtcctacagacccccagtaaaaac

SEQ ID NO: 33 - DNA, 36 bp, Artificial Sequence
  Capture probe
cccagccagaaacgaggcaagagttccccgcggtag

SEQ ID NO: 34 - DNA, 35 bp, Artificial Sequence
  Reporter probe
cagcacggtaggcattctgccgttcgccagcagac

SEQ ID NO: 35 - DNA, 50 bp, Artificial Sequence
  Capture probe
tgtgctgaaactagactgtcaactctgtaagagcttggaccaagtctgtc

SEQ ID NO: 36 - DNA, 50 bp, Artificial Sequence
  Reporter probe
aaacaaagagctcaaggcctcaccttggtttattcactgctggttttcta

SEQ ID NO: 37 - DNA, 50 bp, Artificial Sequence
  Capture probe
tcttgctttttctattgacttgagtttctccttcgcttggtaaacagctg

SEQ ID NO: 38 - DNA, 50 bp, Artificial Sequence
  Reporter probe
ctgatcctatgtgcatacttaattatttcttcagaggagatagcaccttt

SEQ ID NO: 39 - DNA, 35 bp, Artificial Sequence
  Capture probe
gaatgtgcaggtggcatccctgaggattcagagct

SEQ ID NO: 40 - DNA, 42 bp, Artificial Sequence
  Reporter probe
tatcgtggtaaaggctaggctgggaccccggacagagtatga

SEQ ID NO: 41 - DNA, 50 bp, Artificial Sequence
  Capture probe
gatctatgcaacttctgataggactccaactcccttacactgctggaaac

SEQ ID NO: 42 - DNA, 50 bp, Artificial Sequence
  Reporter probe
cctttcagccacagaaacgattgacatgcttctctccccaacccctagaa

SEQ ID NO: 43 - DNA, 50 bp, Artificial Sequence
  Capture probe
tagggctggaacaaggactcttttctctggacagcttgcacctacaatcc

SEQ ID NO: 44 - DNA, 50 bp, Artificial Sequence
  Reporter probe
ccaaaggaatattgcaaatacccaaggtcaccctgtcaggagtggcagaa

SEQ ID NO: 45 - DNA, 45 bp, Artificial Sequence
  Capture probe
tcagtgggtagcgaaaggtgtagaaggcggacatcagggccttgg

SEQ ID NO: 46 - DNA, 41 bp, Artificial Sequence
  Reporter probe
atataggtgttgaacgcccctgcagtcatctgggctgagac

SEQ ID NO: 47 - DNA, 50 bp, Artificial Sequence
  Capture probe
caggatttccagaatttggtaaaaaggcatggcctaagataccacctggc

SEQ ID NO: 48 - DNA, 50 bp, Artificial Sequence
  Reporter probe
tccagtgtctgaagctgaccagtgttcattcttgtcaaaatggacaactc

SEQ ID NO: 49 - DNA, 50 bp, Artificial Sequence
  Capture probe
cactgtgttcaaggcccacttccaccaaaaatctagctgtgtggcctcaa

SEQ ID NO: 50 - DNA, 50 bp, Artificial Sequence
  Reporter probe
gaactcagagagcagacactgggttttacagtcagaaactgcagaaagta

SEQ ID NO: 51 - DNA, 41 bp, Artificial Sequence
  Capture probe
agctgggactggagtgtgaacaaactgtcttccaggttccg

SEQ ID NO: 52 - DNA, 35 bp, Artificial Sequence
  Reporter probe
gctgggcacctgtggaagcactttgcaacagttgc

SEQ ID NO: 53 - DNA, 50 bp, Artificial Sequence
  Capture probe
taaggaacacatcaattcattttctaatgtccttccctcacaagcgggac

SEQ ID NO: 54 - DNA, 50 bp, Artificial Sequence
  Reporter probe
tcccgttcaaataaatatccacaacaggatctgttttcctgcccatcctt

SEQ ID NO: 55 - DNA, 50 bp, Artificial Sequence
  Capture probe
cactttcatccagtcgcctttcagttcccagggccaagaggttattgtat

SEQ ID NO: 56 - DNA, 50 bp, Artificial Sequence
  Reporter probe
aggaggaaactgccttctccaggttgatccacgtctgaagttcttgtcat

SEQ ID NO: 57 - DNA, 46 bp, Artificial Sequence
  Capture probe
ttaatgtaggtcgtgcgcatttgccgggctcggtggcgccgcagcc

SEQ ID NO: 58 - DNA, 50 bp, Artificial Sequence
  Reporter probe
atccgcaagtcggcggcggggtccaattcaaacagctgtctctgcataaa

SEQ ID NO: 59 - DNA, 44 bp, Artificial Sequence
  Capture probe
ttcatggcgatgcccagcttgcccgtgcacagcctctgggagat

SEQ ID NO: 60 - DNA, 50 bp, Artificial Sequence
  Reporter probe
ggtagcccaggatgaagatgatccagaagagggccacgccgcccagcacc

SEQ ID NO: 61 - DNA, 46 bp, Artificial Sequence
  Capture probe
ccacaaggcagggagagaagggagccacataagtagattcctggcg

SEQ ID NO: 62 - DNA, 35 bp, Artificial Sequence
  Reporter probe
atggtaggcatcatgaagggcacagtgctcgctgc

SEQ ID NO: 63 - DNA, 50 bp, Artificial Sequence
  Capture probe
ccctcaattcatattttattcttgagccgcttggtcaggtttgattcgca

SEQ ID NO: 64 - DNA, 47 bp, Artificial Sequence
  Reporter probe
tcagcctccaaattgctgggattacagacatgacctaccgtcccggg

SEQ ID NO: 65 - DNA, 50 bp, Artificial Sequence
  Capture probe
tgtttctagactgtatatctgctaactggcaccgtattccctgaaaggga

SEQ ID NO: 66 - DNA, 50 bp, Artificial Sequence
  Reporter probe
cccagcaacacacatctggaatcttgttatggcttcttcagaccaatgtt

SEQ ID NO: 67 - DNA, 35 bp, Artificial Sequence
  Capture probe
ctgccgcgctccaggcggcgctccccgcccctcgc

SEQ ID NO: 68 - DNA, 50 bp, Artificial Sequence
  Reporter probe
taggcacactcaaacaacgactggtcctcactcacaactgataaggcttc

SEQ ID NO: 69 - DNA, 50 bp, Artificial Sequence
  Capture probe
cttccagtagagatcgctgttgccctgtactttgccgaatgtgtaactga

SEQ ID NO: 70 - DNA, 50 bp, Artificial Sequence
  Reporter probe
gccagcgcgggccgagagtggaattcccggatgaggcggtaacgctgcgc

SEQ ID NO: 71 - DNA, 36 bp, Artificial Sequence
  Capture probe
ctcggcggctgctgccggtctggctcttcctcgctg

SEQ ID NO: 72 - DNA, 35 bp, Artificial Sequence
  Reporter probe
tgctgctgcgccgcttgcgtcccccgcgcttgccg

SEQ ID NO: 73 - DNA, 40 bp, Artificial Sequence
  Capture probe
acgaggtttgtcacctggtatgcactgagccgagtgactg

SEQ ID NO: 74 - DNA, 44 bp, Artificial Sequence
  Reporter probe
tccccttcttcactaggtaggaaatgtagaatttggttcctggc

SEQ ID NO: 75 - DNA, 50 bp, Artificial Sequence
  Capture probe
ccatatacaacaaatccgatatggatccctttcttgccacgggaaggctc

SEQ ID NO: 76 - DNA, 50 bp, Artificial Sequence
  Reporter probe
tctaactagtaagacaggtgggaggttctttgtgaggatttccaaccaag